(19) United States
(12) Patent Application Publication
(10) Pub. No.: US 2021/0010025 A1
(43) Pub. Date: Jan. 14, 2021

(54) TREATMENT OF OCULAR DISEASES WITH HUMAN POST-TRANSLATIONALLY MODIFIED VEGF-TRAP

(71) Applicant: REGENXBIO INC., Rockville, MD (US)

(72) Inventors: Olivier Danos, New York, NY (US); Zhuchun Wu, North Potomac, MD (US); Franz Michael Gerner, Myersville, MD (US); Sherri Van Everen, Menlo Park, CA (US)

(21) Appl. No.: 16/810,422

(22) Filed: Mar. 5, 2020

Related U.S. Application Data
(63) Continuation of application No. PCT/US2018/056343, filed on Oct. 17, 2018.

(51) Int. Cl. C12N 15/86; C07K 14/71; C12N 7/00; A61K 9/00

(52) U.S. Cl. CPC C12N 15/86; C07K 14/71; C12N 7/00; A61K 9/0048; A61K 9/0051; A61K 38/00; C12N 2750/14143; C12N 2800/22; C12N 2830/002; C12N 2830/50; C12N 2750/14151; A61K 9/0019

(57) ABSTRACT

Compositions and methods are described for the delivery of a fully human post-translationally modified (HuPTM) therapeutic VEGF-Trap (VEGF-Trap^{HuPTM})—to a human subject diagnosed with an ocular disease or condition or cancer associated with neovascularization and indicated for treatment with the therapeutic mAb. Delivery may be advantageously accomplished via gene therapy—e.g., by administering a viral vector or other DNA expression construct encoding the VEGF-Trap^{HuPTM} to a patient (human subject) diagnosed with an ocular condition or cancer indicated for treatment with the VEGF-Trap—to create a permanent depot in a tissue or organ of the patient that continuously supplies the VEGF-Trap^{HuPTM}, i.e., a human-glycosylated transgene product. Alternatively, the VEGF-Trap^{HuPTM}, for example, produced in cultured human cell culture, can be administered to the patient for treatment of the ocular disease or cancer.

### CROSS REFERENCE TO RELATED PATENT APPLICATION

This application is a continuation of International Patent Application No. PCT/US2018/056343 filed Oct. 17, 2018, which is herein incorporated by reference in its entirety.

### 0. SEQUENCE LISTING

The instant application contains a Sequence Listing which has been submitted electronically in ASCII format and is hereby incorporated by reference in its entirety. Said ASCII copy, created on Oct. 15, 2018, is named 26115_105002_SL.txt and is 197,438 bytes in size.

### 1. INTRODUCTION

The invention involves compositions and methods for the delivery of a fully human-post-translationally modified (HuPTM) VEGF-Trap (VEGF-Trap^{HuPTM}) to the retina/vitreal humour in the eye(s) of human subjects diagnosed with ocular diseases caused by increased vascularization, including for example, wet age-related macular degeneration (“WAMD”), age-related macular degeneration (“AMD”), diabetic retinopathy, diabetic macular edema (DME), central retinal vein occlusion (RVO), pathologic myopia, and polypoidal choroidal vasculopathy. Also provided are compositions and methods for the delivery of VEGF-Trap^{HuPTM} to a tumor for the treatment of cancer, particularly metastatic colon cancer.

### 2. BACKGROUND OF THE INVENTION

Age-related macular degeneration (AMD) is a degenerative retinal eye disease that causes a progressive, irreversible, severe loss of central vision. The disease impairs the macula—the region of highest visual acuity (VA)—and is the leading cause of blindness in Americans 60 years or older (Hageman et al. Age-Related Macular Degeneration (AMD) 2008 in Kolb et al., eds. Webvision: The Organization of the Retina and Visual System. Salt Lake City (Utah): University of Utah Health Sciences Center; 1995—(available from: https://www.ncbi.nlm.nih.gov/books/NBK27323/)).

The “wet”, neovascular form of AMD (WAMD), also known as neovascular age-related macular degeneration (nAMD), accounts for 15-20% of AMD cases, and is characterized by abnormal neovascularization in and under the neuroretina in response to various stimuli. This abnormal vessel growth leads to formation of leaky vessels and often hemorrhage, as well as distortion and destruction of the normal retinal architecture. Visual function is severely impaired in WAMD, and eventually inflammation and scarring cause permanent loss of visual function in the affected retina. Ultimately, photoreceptor death and scar formation result in a severe loss of central vision and the inability to read, write, and recognize faces or drive. Many patients can no longer maintain gainful employment, carry out daily activities and consequently report a diminished quality of life (Mitchell and Bradley, 2006, Health Qual Life Outcomes 4: 97).

Preventative therapies have demonstrated little effect, and therapeutic strategies have focused primarily on treating the neovascular lesion and associated fluid accumulation. While treatments for WAMD have included laser photocoagulation, and photodynamic therapy with verteporfin, currently, the standard of care treatment for WAMD includes intravitreal (“IVT”) injections with agents aimed at binding to and neutralizing vascular endothelial growth factor (“VEGF”)—a cytokine implicated in stimulating angiogenesis and targeted for intervention. VEGF inhibitors (“anti-VEGF” agents) used include, e.g., ranibizumab (a small anti-VEGF Fab protein which was affinity-improved and made in prokaryotic E. coli); off-label bevacizumab (a humanized monoclonal antibody (mAb) against VEGF produced in CHO cells); or aflibercept (a recombinant fusion protein consisting of VEGF-binding regions of the extracellular domains of the human VEGF-receptor fused to the Fc portion of human IgG₁, belonging to a class of molecules commonly known as “VEGF-Traps”). Each of these therapies have improved best-corrected visual acuity on average in naïve WAMD patients; however, their effects appear limited in duration and patients usually receive frequent doses every 4 to 6 weeks on average.

Frequent IVT injections create considerable treatment burden for patients and their caregivers. While long term therapy slows the progression of vision loss and improves vision on average in the short term, none of these treatments prevent neovascularization from recurring (Brown, 2006, N Engl J Med 355:1432-1444; Rosenfeld, 2006 N Engl J Med 355:1419-1431; Schmidt-Erfurth, 2014, Ophthalmology 121(1): 193-201). Each must be re-administered to prevent the disease from worsening. The need for repeat treatments can incur additional risk to patients and is inconvenient for both patients and treating physicians.

A related VEGF-trap, viz-aflibercept (which has the amino acid sequence of aflibercept in a formulation unsuitable for administration to the eye) is used for the treatment of metastatic colon cancer and dosed by a one hour intravenous infusion every two weeks. The half-life ranges from 4 to 7 days and repeat administration is required. Dose limiting side effects, such as hemorrhage, gastrointestinal perforation and compromised wound healing can limit therapeutic effect. See Bender et al., 2012, Clin. Cancer Res. 18:5081.

### 3. SUMMARY OF THE INVENTION

Compositions and methods are provided for the delivery of a human-post-translationally modified VEGF-Trap (VEGF-Trap^{HuPTM}) to the retina/vitreal humour in the eye(s) of patients (human subjects) diagnosed with an ocular disease caused by increased vascularization, for example, nAMD, also known as “wet” AMD. This may be accomplished via gene therapy—e.g., by administering a viral vector or other DNA expression construct encoding (as a transgene) a VEGF-Trap protein to the eye(s) of patients (human subjects) diagnosed with nAMD, or other ocular disease caused by vascularization, to create a permanent depot in the eye that continuously supplies the fully human post-translationally modified transgene product. Such DNA vectors can be administered to the subretinal space, or to the suprachoroidal space, or intravitreally to the patient. The VEGF-Trap^{HuPTM} may have fully human post-translational modifications due to expression in human cells (as compared to non-human CHO cells). The method can be used to treat any ocular indication that responds to VEGF inhibition, especially those that respond to aflibercept (EYLEA®): e.g., AMD, diabetic retinopathy, diabetic macular edema (DME), including diabetic retinopathy in patients with DME, central retinal vein occlusion (RVO) and macular edema following RVO, pathologic myopia, particularly as caused by myopic choroidal neovascularization, and polypoidal choroidal vasculopathy, to name a few.

In other embodiments, provided are compositions and methods for delivery of a VEGF-Trap^{HuPTM} to cancer cells and surrounding tissue, particularly tissue exhibiting increased vascularization, in patients diagnosed with cancer, for example, metastatic colon cancer. This may be accomplished via gene therapy—e.g., by administering a viral vector or other DNA expression construct encoding as a transgene a VEGF-Trap protein to the liver of patients (human subjects) diagnosed with cancer, particularly metastatic colon cancer, to create a permanent depot in the liver that continuously supplies the fully human post-translationally modified transgene product. Such DNA vectors can be administered intravenously to the patient, or directly to the liver through hepatic blood flow, e.g., via the suprahepatic veins or via the hepatic artery.

The VEGF-Trap^{HuPTM} encoded by the transgene is a fusion protein which comprises (from amino to carboxy terminus): (i) the Ig-like domain 2 of Flt-1 (human; also named VEGFR1), (ii) the Ig-like domain 3 of KDR (human; also named VEGFR2), and (iii) a human IgG Fc region, particularly a IgG1 Fc region. In specific embodiments, the VEGF-Trap^{HuPTM} has the amino acid sequence of aflibercept (SEQ ID NO: 1 and FIG. 1, which provide the numbering of the amino acid positions in FIG. 1 will be used herein; see also Table 1, infra for amino acid sequence of aflibercept and codon optimized nucleotide sequences encoding aflibercept). FIG. 1 also provides the Flt-1 leader sequence at the N-terminus of the aflibercept sequence, and the transgene may include the sequence coding for the leader sequence of FIG. 1 or other alternate leader sequences as disclosed infra. Alternatively, the transgene may encode variants of a VEGF-Trap designed to increase stability and residence in the eye, yet reduce the systemic half-life of the transgene product following entry into the systemic circulation; truncated or “Fc-less” VEGF-Trap constructs, VEGF Trap transgenes with a modified Fc, wherein the modification disables the FcRn binding site and or where another Fc region or Ig-like domain is substituted for the IgG1 Fc domain.

In certain aspects, provided herein are constructs for the expression of VEGF-Trap transgenes in human retinal cells. The constructs can include expression vectors comprising nucleotide sequences encoding a transgene and appropriate expression control elements for expression in retinal cells. The recombinant vector used for delivering the transgene to retinal cells should have a tropism for retinal cells. In other aspects, provided are constructs for the expression of the VEGF-Trap transgenes in human liver cells and these constructs can include expression vectors comprising nucleotide sequences encoding a transgene and appropriate expression control elements for expression in human liver cells. The recombinant vector used for delivering the transgene to the liver should have a tropism for liver cells. These vectors can include non-replicating recombinant adeno-associated virus vectors (“rAAV”), particularly those bearing an AAV8 capsid, or variants of an AAV8 capsid are preferred. However, other viral vectors may be used, including but not limited to lentiviral vectors, vaccinia viral vectors, or non-viral expression vectors referred to as “naked DNA” constructs. Preferably, the VEGF-Trap^{HuPTM} transgene should be controlled by appropriate expression control elements, for example, the ubiquitous CB7 promoter (a chicken β-actin promoter and CMV enhancer), or tissue-specific promoters such as RPE-specific promoters e.g., the RPE65 promoter, or cone-specific promoters, e.g., the opsin promoter, or liver specific promoters such as the TBG (Thyroxine-binding Globulin) promoter, the APOA2 promoter, the SERPINA1 (hAAT) promoter or the MIR122 promoter. In certain embodiments, particularly for cancer indications, inducible promoters may be preferred so that transgene expression may be turned on and off as desired for therapeutic efficacy. Such promoters include, for example, hypoxia-induced promoters and drug inducible promoters, such as promoters induced by rapamycin and related agents. Hypoxia-inducible promoters include promoters with HIF binding sites, see for example, Schödel, et al., Blood, 2011, 117(23):e207-e217 and Kenneth and Rocha, Biochem J., 2008, 414:19-29, each of which is incorporated by reference for teachings of hypoxia-inducible promoters. In addition, hypoxia-inducible promoters that may be used in the constructs include the erythropoietin promoter and N-WASP promoter (see, Tsuchiya, 1993, J. Biochem. 113:395 for disclosure of the erythropoietin promoter and Salvi, 2017, Biochemistry and Biophysics Reports 9:13-21 for disclosure of N-WASP promoter, both of which are incorporated by reference for the teachings of hypoxia-induced promoters). Alternatively, the constructs may contain drug inducible promoters, for example promoters inducible by administration of rapamycin and related analogs (see, for example, International Publications WO94/18317, WO 96/20951, WO 96/41865, WO 99/10508, WO 99/10510, WO 99/36553, and WO 99/41258, and U.S. Pat. No. 7,067,526 (disclosing rapamycin analogs), which are incorporated by reference herein for their disclosure of drug inducible promoters).

The construct can include other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor /splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal). See, e.g., Powell and Rivera-Soto, 2015, Discov. Med., 19(102):49-57.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161). Provided as SEQ ID NO: 2 is a codon optimized nucleotide sequence that encodes the transgene product of SEQ ID NO: 1, plus the leader sequence provided in FIG. 1. SEQ ID NO: 3 is a consensus codon optimized nucleotide sequence encoding the transgene product of SEQ ID NO: 1 plus the leader sequence in FIG. 1 (see Table 1, infra, for SEQ ID NOs: 2 and 3).

In specific embodiments, provided are constructs for gene therapy administration for treating ocular disorders, including macular degeneration (nAMD), diabetic retinopathy, diabetic macular edema (DME), central retinal vein occlusion (RVO), pathologic myopia, or polypoidal choroidal vasculopathy, in a human subject in need thereof, comprising an AAV vector, which comprises a viral capsid that is at least 95% identical to the amino acid sequence of an AAV8 capsid (SEQ ID NO: 11); and a viral genome comprising an expression cassette flanked by AAV inverted terminal repeats (ITRs) wherein the expression cassette comprises a transgene encoding a VEGF-Trap^{HuPTM}, operably linked to one or more regulatory sequences that control expression of the transgene in human retinal cells. In specific embodiments, provided are constructs for gene therapy administration for treating cancer, particularly metastatic colon cancer, in a human subject in need thereof, comprising an AAV vector, which comprises a viral capsid that is at least 95% identical to the amino acid sequence of an AAV8 capsid (SEQ ID NO: 11); and a viral genome comprising an expression cassette flanked by AAV inverted terminal repeats (ITRs) wherein the expression cassette comprises a transgene encoding a VEGF-Trap^{HuPTM}, operably linked to one or more regulatory sequences that control expression of the transgene in human liver cells. In certain embodiments, the encoded AAV8 capsid has the sequence of SEQ ID NO: 11 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions, particularly substitutions with amino acid residues found in the corresponding position in other AAV capsids, for example, as shown in FIG. 6 which provides a comparison of the amino acid sequences of the capsid sequences of various AAVs, highlighting amino acids appropriate for substitution at different positions within the capsid sequence in the row labeled “SUBS”.

In certain embodiments, the VEGF-Trap^{HuPTM} encoded by the transgene has the amino acid sequence of aflibercept (SEQ ID NO:1). In certain embodiments, the VEGF-Trap^{HuPTM} is a variant of SEQ ID NO: 1 that has modifications to the IgG1 Fc domain that may reduce the half-life of the VEGF-Trap^{HuPTM} in the systemic circulation while maintaining the stability in the eye. Provided herein is a VEGF-Trap^{HuPTM} that does not comprise the IgG1 Fc domain (Fc-less or Fc⁽⁻⁾ variant), for example, as set forth in FIG. 4. In specific embodiments, the VEGF-Trap^{HuPTM} may or may not contain the terminal lysine of the KDKsequence (i.e., amino acid 205 in FIG. 4) depending upon carboxypeptidase activity. Alternatively, the VEGF-Trap^{HuPTM} may have all or a portion of the hinge region of IgG1 Fc at the C-terminus of the protein, as shown in FIG. 4, the C-terminal sequence may be KDKTHT (SEQ ID NO: 31) OR KDKTHL(SEQ ID NO: 32), KDKTHTCPPCPA(SEQ ID NO: 33), KDKTHTCPPCPAPELLGG (SEQ ID NO: 34), or KDKTHTCPPCPAPELLGGPSVFL(SEQ ID NO: 35). The cysteine residues in the hinge region may promote the formation of inter-chain disulfide bonds whereas fusion proteins that do not contain all or a cysteine-containing portion of the hinge region may not form inter chain bonds but only intra-chain bonds.

Alternatively, in other embodiments, the VEGF-Trap^{HuPTM} has mutations in the IgG1 Fc domain that reduce FcRn binding and, thereby, the systemic half-life of the protein (Andersen, 2012, J Biol Chem 287: 22927-22937). These mutations include mutations at I253, H310, and/or H435 and, more specifically, include I253A, H310A, and/or H435Q or H435A, using the usual numbering of the positions in the IgG1 heavy chain. These positions correspond to I238, H295 and H420 in the VEGF-Trap^{HuPTM} of SEQ ID NO: 1 (and in FIG. 1 in which the positions are highlighted in pink). Thus, provided is a VEGF-Trap^{HuPTM} comprising an IgG1 Fc domain with one, two or three of the mutations I238A, H295A and H420Q or H420A. An exemplary VEGF-Trap^{HuPTM} amino acid sequence of a fusion protein having the amino acid sequence of aflibercept with an alanine or glutamine substitution for histidine at position 420 is provided in FIG. 3.

In alternative embodiments, the VEGF-Trap^{HuPTM} has an Fc domain or other domain sequence substituted for the IgG1 Fc domain that may improve or maintain the stability of the VEGF-Trap^{HuPTM} in the eye while reducing the half-life of the VEGF-Trap^{HuPTM} once it has entered the systemic circulation, reducing the potential for adverse effects. In particular embodiments, the VEGF-Trap^{HuPTM} has substituted for the IgG1 domain an alternative Fc domain, including an IgG2 Fc or IgG4 Fc domain, as set forth in FIGS. 7A and B, respectively, where the hinge sequence is indicated in italics. Variants include all or a portion of the hinge region, or none of the hinge region. In those variants having a hinge region, the hinge region sequence may also have one or two substitutions of a serine for a cysteine in the hinge region such that interchain disulfide bonds do not form. The amino acid sequences of exemplary transgene products are presented in FIGS. 7C-H.

In other alternative embodiments, the VEGF-Trap^{HuPTM} has substituted for the IgG1 Fc domain, one or more of the Ig-like domains of Flt-1 or KDR, or a combination thereof. The amino acid sequences of the extracellular domains of human Flt 1 and human KDR are presented in FIGS. 8A and 8B, respectively, with the Ig-like domains indicated in color text. Provided are transgene products in which the C-terminal domain consists of or comprises one, two, three or four of the Ig-like domains of Flt1, particularly, at least the Ig-like domains 2 and 3; or one, two, three or four of the Ig-like domains of KDR, particularly, at least domains 3, 4, and/or 5. In a specific embodiment, the transgene product has a C-terminal domain with the KDR Ig-like domains 3, 4 and 5 and the Flt1 Ig-like domain 2. The amino acid sequences of exemplary transgene products are provided in FIGS. 8C and D.

The construct for the VEGF-Trap^{HuPTM} should include a nucleotide sequence encoding a signal peptide that ensures proper co- and post-translational processing (glycosylation and protein sulfation) by the transduced retinal cells or liver cells. In some embodiments, the signal sequence is that of Flt-1, MVSYWDTGVLLCALLSCLLLTGSSSG (SEQ ID NO: 36) (see FIG. 1). In alternative embodiments, the signal sequence is the KDR signal sequence, MQSKVLLAVALWLCVETRA (SEQ ID NO: 37), or alternatively, in a preferred embodiment, MYRMQLLLLIALSLALVTNS (SEQ ID NO: 38) (FIG. 2) or MRMQLLLLIALSLALVTNS (SEQ ID NO: 39). Other signal sequences used for expression in human retinal cells may include, but are not limited to, those in Table 3, infra, and signal sequences used for expression in human liver cells may include, but are not limited to, those in Table 4, infra.

In specific embodiments, the VEGF-Trap^{HuPTM} has the amino acid sequence set forth in FIG. 1, FIG. 2, FIG. 3, FIG. 4, FIGS. 7C-7H or FIGS. 8C and 8D.

In specific embodiments, provided are constructs that encode two copies of a fusion protein having the amino acid sequence of the Ig-like Domain 2 of Flt-1 and the Ig-like domain 3 of KDR (i.e., the amino acid sequence of aflibercept without the IgG1 Fc domain (but may include all or a portion of the hinge region of the IgG1 Fc domain (see FIG. 4) by linking identical copies of the sequences with either a flexible or rigid short peptide as a linker, including rigid linkers such as (GP)ₙ (SEQ ID NO: 40) or (AP)ₙ (SEQ ID NO: 41) or (EAAAK)₃(SEQ ID NO: 42), or flexible linker such as (GGGGS)ₙ (SEQ ID NO: 43), where for any of these n=1, 2, 3, or 4 (Chen, 2013, “Fusion protein linkers: property, design and functionality”, Adv. Drug. Deliv. 65(10): 1357-1369, at Table 3). The construct may be arranged as: Leader-FM Ig-like Domain 2-KDR-Ig-like Domain 3+linker+Flt-1 Ig-like Domain 2-KDR (Ig-like Domain 3). Alternatively, the construct is bicistronic with two copies of the Fc-less VEGF-Trap transgene with an IRES sequence between the two to promote separate expression of the second copy of the Fc-less VEGF-Trap protein.

In a specific embodiment, the constructs described herein comprise the following components: (1) AAV2 inverted terminal repeats that flank the expression cassette; (2) Control elements, which include a) the CB7 promoter, comprising the CMV enhancer/chicken β-actin promoter, b) a chicken β-actin intron and c) a rabbit β-globin poly A signal; and (3) nucleotide sequences coding for the VEGF-Trap^{HuPTM} as described above.

In a specific embodiment, the constructs described herein comprise the following components: (1) AAV2 inverted terminal repeats that flank the expression cassette; (2) Control elements, which include a) a hypoxia-inducible promoter, b) a chicken β-actin intron and c) a rabbit β-globin poly A signal; and (3) nucleotide sequences coding for the VEGF-Trap^{HuPTM} as described above.

In certain aspects, described herein are methods of treating a human subject diagnosed with neovascular age-related macular degeneration (nAMD), diabetic retinopathy, diabetic macular edema (DME), central retinal vein occlusion (RVO), pathologic myopia, or polypoidal choroidal vasculopathy, comprising delivering to the retina of said human subject a therapeutically effective amount of a VEGF-Trap^{HuPTM} produced by human retinal cells.

In certain aspects, described herein are methods of treating a human subject diagnosed with nAMD, diabetic retinopathy, DME, cRVO, pathologic myopia, or polypoidal choroidal vasculopathy, comprising delivering to the retina of said human subject a therapeutically effective amount of a VEGF-Trap^{HuPTM} produced by one or more of the following retinal cell types: human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells.

In certain aspects, described herein are methods of treating a human subject diagnosed with cancer, particularly metastatic colon cancer, comprising delivering to the cancer cells or surrounding tissue (e.g., the tissue exhibiting increased vascularization surrounding the cancer cells) of said human subject a therapeutically effective amount of a VEGF-Trap^{HuPTM} produced by human liver cells.

In certain aspects of the methods described herein, the VEGF-Trap^{HuPTM} is a protein comprising the amino acid sequence of FIG. 1, FIG. 2, FIG. 3, FIG. 4, FIG. 7C, FIG. 7D, FIG. 7E, FIG. 7F, FIG. 7G, FIG. 7H, FIG. 8C, or FIG. 8D (either including or excluding the leader sequence at the N-terminus presented).

In certain aspects, described herein are methods of treating a human subject diagnosed with nAMD, diabetic retinopathy, DME, cRVO, pathologic myopia, or polypoidal choroidal vasculopathy, comprising: delivering to the eye of said human subject, a therapeutically effective amount of a VEGF-Trap^{HuPTM}, said VEGF-Trap^{HuPTM} containing α2,6-sialylated glycans.

In certain aspects, described herein are methods of treating a human subject diagnosed with nAMD, diabetic retinopathy, DME, cRVO, pathologic myopia, or polypoidal choroidal vasculopathy, comprising: delivering to the eye of said human subject, a therapeutically effective amount of a glycosylated VEGF-Trap^{HuPTM}, wherein said VEGF-Trap does not contain NeuGc (i.e. levels detectable by standard assays described infra).

In certain aspects, described herein are methods of treating a human subject diagnosed with nAMD, diabetic retinopathy, DME, cRVO, pathologic myopia, or polypoidal choroidal vasculopathy, comprising: delivering to the eye of said human subject, a therapeutically effective amount of a glycosylated VEGF-Trap^{HuPTM}, wherein said VEGF-Trap does not contain detectable levels of the α-Gal epitope (i.e. levels detectable by standard assays described infra).

In certain aspects, described herein are methods of treating a human subject diagnosed with nAMD, diabetic retinopathy, DME, cRVO, pathologic myopia, or polypoidal choroidal vasculopathy, comprising: delivering to the eye of said human subject, a therapeutically effective amount of a glycosylated VEGF-Trap^{HuPTM}, wherein said VEGF-Trap does not contain NeuGc or α-Gal.

In certain aspects, described herein are methods of treating a human subject diagnosed with nAMD, diabetic retinopathy, DME, cRVO, pathologic myopia, or polypoidal choroidal vasculopathy, wherein the method comprises: administering to the subretinal space,or intravitreally or suprachoroidally, in the eye of said human subject an expression vector encoding a VEGF-Trap^{HuPTM}, wherein said VEGF-Trap^{HuPTM} is α2,6-sialylated upon expression from said expression vector in a human, immortalized retina-derived cell.

In certain aspects, described herein are methods of treating a human subject diagnosed with nAMD, diabetic retinopathy, DME, cRVO, pathologic myopia, or polypoidal choroidal vasculopathy, wherein the method comprises: administering to the subretinal space, or intravitreally or suprachoroidally, in the eye of said human subject an expression vector encoding an a VEGF-Trap^{HuPTM}, wherein said VEGF-Trap is α2,6-sialylated but does not contain NeuGc and/or α-Gal upon expression from said expression vector in a human, immortalized retina-derived cell.

In certain aspects, described herein are methods of treating a human subject diagnosed with metastatic colon cancer, comprising: administering to the liver of said human subject, a therapeutically effective amount of a recombinant nucleotide expression vector encoding a VEGF-Trap^{HuPTM}, so that a depot is formed that releases said VEGF-Trap^{HuPTM} containing α2,6-sialylated glycans.

In certain aspects, described herein are methods of treating a human subject diagnosed with metastatic colon cancer, comprising: administering to the liver of said human subject, a therapeutically effective amount of a recombinant nucleotide expression vector encoding a VEGF-Trap^{HuPTM}, so that a depot is formed that releases said VEGF-Trap^{HuPTM} which is glycosylated but does not contain NeuGc and/or α-Gal.

In certain aspects, described herein are methods of treating a human subject diagnosed with metastatic colon cancer, comprising: delivering to cancer cells and/or surrounding tissue of said cancer cells of said human subject, a therapeutically effective amount of a VEGF-Trap^{HuPTM}, said VEGF-Trap^{HuPTM} containing α2,6-sialylated glycans.

In certain aspects, described herein are methods of treating a human subject diagnosed with metastatic colon cancer, comprising: delivering to cancer cells and/or surrounding tissue of said cancer cells of said human subject, a therapeutically effective amount of a VEGF-Trap^{HuPTM}, wherein said VEGF-Trap^{HuPTM} does not contain NeuGc.

In certain aspects, described herein are methods of treating a human subject diagnosed with metastatic colon cancer, comprising: delivering to cancer cells and/or surrounding tissue of said cancer cells of said human subject, a therapeutically effective amount of a VEGF-Trap^{HuPTM}, wherein said VEGF-Trap^{HuPTM} does not contain α-Gal.

In certain aspects, described herein are methods of treating a human subject diagnosed with metastatic colon cancer, comprising: delivering to cancer cells and/or surrounding tissue of said cancer cells of said human subject, a therapeutically effective amount of a VEGF-Trap^{HuPTM}, wherein said VEGF-Trap^{HuPTM} does not contain NeuGc or α-Gal.

In certain aspects, described herein are methods of treating a human subject diagnosed with metastatic colon cancer, wherein the method comprises: administering to the liver of said human subject an expression vector encoding a VEGF-Trap^{HuPTM}, wherein said VEGF-Trap^{HuPTM} is α2,6-sialylated upon expression from said expression vector in a human, immortalized liver-derived cell.

In certain aspects, described herein are methods of treating a human subject diagnosed with metastatic colon cancer, wherein the method comprises: administering to the liver of said human subject an expression vector encoding an a VEGF-Trap^{HuPTM}, wherein said VEGF-Trap^{HuPTM} is α2,6-sialylated but does not contain detectable NeuGc and/or α-Gal upon expression from said expression vector in a human, immortalized liver-derived cell.

In certain aspects of the methods described herein, the VEGF-Trap^{HuPTM} comprises the amino acid sequence of FIG. 1, FIG. 2, FIG. 3, FIG. 4, FIG. 7C, FIG. 7D, FIG. 7E, FIG. 7F, FIG. 7G, FIG. 7H, FIG. 8C, or FIG. 8D (either including the leader sequence presented in the Figure or an alternate leader sequence or no leader sequence).

In certain aspects of the methods described herein, the VEGF-Trap^{HuPTM} further contains a tyrosine-sulfation.

In certain aspects of the methods described herein, production of said VEGF-Trap^{HuPTM} containing a α2,6-sialylated glycan is confirmed by transducing PER.C6 or RPE cell line with said recombinant nucleotide expression vector in cell culture and expressing said VEGF-Trap^{HuPTM}.

In certain aspects of the methods described herein, production of said VEGF-Trap^{HuPTM} containing a tyrosine-sulfation is confirmed by transducing PER.C6 or RPE cell line with said recombinant nucleotide expression vector in cell culture.

In certain aspects of the methods described herein, the VEGF-Trap^{HuPTM} transgene encodes a leader peptide. A leader peptide may also be referred to as a signal peptide or leader sequence herein.

In certain aspects, described herein are methods of treating a human subject diagnosed with nAMD, diabetic retinopathy, DME, cRVO, pathologic myopia, or polypoidal choroidal vasculopathy, comprising: administering to the subretinal space, or intravitreally or suprachoroidally, in the eye of said human subject, a therapeutically effective amount of a recombinant nucleotide expression vector encoding a VEGF-Trap^{HuPTM}, so that a depot is formed that releases said VEGF-Trap^{HuPTM} containing a α2,6-sialylated glycan; wherein said recombinant vector, when used to transduce PER.C6 or RPE cells in culture results in production of said VEGF-Trap^{HuPTM} containing a α2,6-sialylated glycan in said cell culture.

In certain aspects, described herein are methods of treating a human subject diagnosed with nAMD, diabetic retinopathy, DME, cRVO, pathologic myopia, or polypoidal choroidal vasculopathy, comprising: administering to the subretinal space, or intravitreally or suprachoroidally, in the eye of said human subject, a therapeutically effective amount of a recombinant nucleotide expression vector encoding a VEGF-Trap^{HuPTM}, so that a depot is formed that releases said VEGF-Trap^{HuPTM} wherein said VEGF-Trap^{HuPTM} is glycosylated but does not contain NeuGc; wherein said recombinant vector, when used to transduce PER.C6 or RPE cells in culture results in production of said VEGF-Trap^{HuPTM} that is glycosylated but does not contain detectable NeuGc and/or α-Gal in said cell culture.

In certain aspects of the methods described herein, delivering to the eye comprises delivering to the retina, choroid, and/or vitreous humor of the eye.

Subjects to whom such gene therapy is administered should be those responsive to anti-VEGF therapy. In particular embodiments, the methods encompass treating patients who have been diagnosed with nAMD, diabetic retinopathy, DME, cRVO, pathologic myopia, or polypoidal choroidal vasculopathy, and identified as responsive to treatment with a VEGF-Trap protein or other anti-VEGF agent. In more specific embodiments, the patients are responsive to treatment with a VEGF-Trap^{HuPTM} protein. In certain embodiments, the patients have been shown to be responsive to treatment with a VEGF-Trap injected intravitreally prior to treatment with gene therapy. In specific embodiments, the patients have previously been treated with aflibercept and have been found to be responsive to aflibercept. In an alternate embodiment, the patients have previously been treated with ranibizumab and have been found to be responsive to ranibizumab. In an alternate embodiment, the patients have previously been treated with bevacizumab and have been found to be responsive to bevacizumab.

Subjects to whom such viral vector or other DNA expression construct is delivered should be responsive to the VEGF-Trap^{HuPTM} encoded by the transgene in the viral vector or expression construct. To determine responsiveness, the VEGF-Trap^{HuPTM} transgene product (e.g., produced in cell culture, bioreactors, etc.) may be administered directly to the subject, such as by intravitreal injection.

In particular embodiments, the methods encompass treating patients who have been diagnosed with metastatic colon cancer, and identified as responsive to treatment with an anti-VEGF agent, particularly a VEGF-Trap protein. In more specific embodiments, the patients are responsive to treatment with a VEGF-Trap^{HuPTM} protein. In certain embodiments, the patients have been shown to be responsive to treatment with a VEGF-Trap administered intravenously prior to treatment with gene therapy. In specific embodiments, the patients have previously been treated with ziv-aflibercept and have been found to be responsive to ziv-aflibercept. In an alternate embodiment, the patients have previously been treated with bevacizumab and have been found to be responsive to bevacizumab. In an alternate embodiment, the patients have previously been treated with ranibizumab and have been found to be responsive to ranibizumab. In an alternate embodiment, the patients have previously been treated with regorafenib and have been found to be responsive to regorafenib.

Subjects to whom such viral vector or other DNA expression construct is delivered should be responsive to the VEGF-Trap^{HuPTM} encoded by the transgene in the viral vector or expression construct. To determine responsiveness, the VEGF-Trap^{HuPTM} transgene product (e.g., produced in cell culture, bioreactors, etc.) may be administered directly to the subject, such as by intravenous infusion.

In certain aspects, provided herein are VEGF-Trap proteins that contain human post-translational modifications. In one aspect, the VEGF-Trap proteins described herein contains the human post-translational modification of α2,6-sialylated glycans. In certain embodiments, the VEGF-Trap proteins only contain human post-translational modifications. In one embodiment, the VEGF-Trap proteins described herein do not contain detectable levels of the immunogenic non-human post-translational modifications of Neu5Gc and/or α-Gal. In another aspect, the VEGF-Trap proteins contain tyrosine (“Y”) sulfation sites. In one embodiment the tyrosine sites are sulfated in the Flt-1 Ig-like domain, the KDR Ig-like domain 3, and/or Fc domain of aflibercept (see FIG. 1 for sulfation sites, highlighted in red). In another aspect, the VEGF-Trap proteins contain α2,6-sialylated glycans and at least one sulfated tyrosine site. In other aspects, the VEGF-Trap proteins contain fully human post-translational modifications (VEGF-Trap^{HuPTM}). In certain aspects, the post-translational modifications of the VEGF-Trap can be assessed by transducing PER.C6 or RPE cells in culture with the transgene, which can result in production of said VEGF-Trap that is glycosylated but does not contain NeuGc in said cell culture. Alternatively, or in addition, the production of said VEGF-Trap containing a tyrosine-sulfation can confirmed by transducing PER.C6 or RPE cell line with said recombinant nucleotide expression vector in cell culture.

Therapeutically effective doses of the recombinant vector should be administered to the eye, e.g., to the subretinal space, or to the suprachoroidal space, or intravitreally in an injection volume ranging from ≥0.1 mL to ≤0.5 mL, preferably in 0.1 to 0.25 mL (100-250 μl). Doses that maintain a concentration of the transgene product that is detectable at a Cₘᵢₙ of at least about 0.33 μg/mL to about 1.32 μg/mL in the vitreous humour, or about 0.11 μg/mL to about 0.44 μg/mL in the aqueous humour (the anterior chamber of the eye) is desired; thereafter, vitreous Cₘᵢₙ concentrations of the transgene product ranging from about 1.70 to about 6.60 μg/mL and up to about 26.40 μg/mL, and/or aqueous Cₘᵢₙ concentrations ranging from about 0.567 to about 2.20 μg/mL, and up to 8.80 μg/mL should be maintained. Vitreous humour concentrations can be estimated and/or monitored by measuring the patient's aqueous humour or serum concentrations of the transgene product. Alternatively, doses sufficient to achieve a reduction in free-VEGF plasma concentrations to about 10 pg/mL can be used. (E.g., see, Avery et al., 2017, Retina, the Journal of Retinal and Vitreous Diseases 0:1-12; and Avery et al., 2014, Br J Ophthalmol 98:1636-1641 each of which is incorporated by reference herein in its entirety).

For treatment of cancer, particularly metastatic colon cancer, therapeutically effective doses should be administered to the patient, preferably intravenously, such that plasma concentrations of the VEGF-Trap transgene product are maintained, after two weeks or four weeks at levels at least the Cₘᵢₙ plasma concentrations of ziv-aflibercept when administered at a dose of 4 mg/kg every two weeks.

The invention has several advantages over standard of care treatments that involve repeated ocular injections of high dose boluses of the VEGF inhibitor that dissipate over time resulting in peak and trough levels. Sustained expression of the transgene product VEGF-Trap, as opposed to injecting a VEGF-Trap product repeatedly, allows for a more consistent levels of the therapeutic to be present at the site of action, and is less risky and more convenient for patients, since fewer injections need to be made, resulting in fewer doctor visits. Furthermore, VEGF-Traps expressed from transgenes are post-translationally modified in a different manner than those that are directly injected because of the different microenvironment present during and after translation. Without being bound by any particular theory, this results in VEGF-Trap molecules that have different diffusion, bioactivity, distribution, affinity, pharmacokinetic, and immunogenicity characteristics, such that the antibodies delivered to the site of action are “biobetters” in comparison with directly injected VEGF-Traps.

In addition, VEGF-Traps expressed from transgenes in vivo are not likely to contain degradation products associated with proteins produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

The invention is based, in part, on the following principles:
(i) Human retinal cells are secretory cells that possess the cellular machinery for post-translational processing of secreted proteins—including glycosylation and tyrosine-O-sulfation, a robust process in retinal cells. (See, e.g., Wang et al., 2013, Analytical Biochem. 427: 20-28 and Adamis et al., 1993, BBRC 193: 631-638 reporting the production of glycoproteins by retinal cells; and Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells, each of which is incorporated by reference in its entirety for post-translational modifications made by human retinal cells).
(ii) Human hepatocytes are secretory cells that possess the cellular machinery for post-translational processing of secreted proteins—including glycosylation and tyrosine-O-sulfation. (See, e.g. https://www.proteinatlas.org/humanproteome/liver for a proteomic identification of plasma proteins secreted by human liver; Clerc et al., 2016, Glycoconj 33:309-343 and Pompach et al. 2014 J Proteome Res. 13:5561-5569 for the spectrum of glycans on those secreted proteins; and E Mishiro, 2006, J Biochem 140:731-737 reporting that TPST-2 (which catalyzes tyrosine-O-sulfation) is more strongly expressed in liver than in other tissues, whereas TPST-1 was expressed in a comparable average level to other tissues, each of which is incorporated by reference in its entirety herein).
(iii) The VEGF-Trap, aflibercept, is a dimeric glycoprotein made in CHO cells with a protein molecular weight of 96.9 kilo Daltons (kDa). It contains approximately 15% glycosylation to give a total molecular weight of 115 kDa. All five putative N-glycosylation sites on each polypeptide chain predicted by the primary sequence can be occupied with carbohydrate and exhibit some degree of chain heterogeneity, including heterogeneity in terminal sialic acid residues. The Fc domain contains a site that is sialylated but at a relatively low level, for example 5 to 20% of the molecules depending upon cell conditions. These N-glycosylation sites are found at positions 36, 68, 123, 196, and 282 of the amino acid sequence in SEQ ID NO:1 (see also FIG. 1 with residues highlighted in yellow). In contrast to ranibizumab and bevacizumab which bind only VEGFA, aflibercept binds all isoforms of VEGF as well as placental growth factor (“PLGF”).
(iv) Unlike CHO-cell products, such as aflibercept, glycosylation of VEGF-Trap^{HuPTM} by human retinal or human liver cells will result in the addition of glycans that can improve stability, half-life and reduce unwanted aggregation of the transgene product. (See, e.g., Bovenkamp et al., 2016, J. Immunol. 196: 1435-1441 for a review of the emerging importance of glycosylation in antibodies and Fabs). Significantly, the glycans that are added to VEGF-Trap^{HuPTM} of the invention are highly processed complex-type N-glycans that contain 2,6-sialic acid. Such glycans are not present in aflibercept which is made in CHO cells that do not have the 2,6-sialyltransferase required to make this post-translational modification, nor do CHO cells produce bisecting GlcNAc, although they do produce Neu5Gc (NGNA), which is immunogenic. See, e.g., Dumont et al., 2015, Critical Rev in Biotech, 36(6):1110-1122. Moreover, CHO cells can also produce an immunogenic glycan, the α-Gal antigen, which reacts with anti-α-Gal antibodies present in most individuals, which at high concentrations can trigger anaphylaxis. See, e.g., Bosques, 2010, Nat Biotech 28: 1153-1156. The human glycosylation pattern of the VEGF-Trap^{HuPTM} of the invention should reduce immunogenicity of the transgene product and improve safety and efficacy.
(v) In addition to the glycosylation sites, VEGF-Traps such as aflibercept may contain tyrosine (“Y”) sulfation sites; see FIG. 1 which highlights in red tyrosine-O-sulfation sites in the Flt-1 Ig-like domain 2, the KDR Ig-like domain 3, and Fc domain of aflibercept. (See, e.g., Yang et al., 2015, Molecules 20:2138-2164, esp. at p. 2154 which is incorporated by reference in its entirety for the analysis of amino acids surrounding tyrosine residues subjected to protein tyrosine sulfation). The “rules” can be summarized as follows: Y residues with E or D within +5 to −5 position of Y, and where position −1 of Y is a neutral or acidic charged amino acid—but not a basic amino acid, e.g., R, K, or H that abolishes sulfation). Sulfation sites may be found at positions 11, 140, 263 and 281 of the VEGF trap sequence of SEQ ID NO:1.
(vi) Tyrosine-sulfation—a robust post-translational process in human retinal cells—could result in transgene products with increased avidity for VEGF. For example, tyrosine-sulfation of the Fab of therapeutic antibodies has been shown to dramatically increase avidity for antigen and activity. (See, e.g., Loos et al., 2015, PNAS 112: 12675-12680, and Choe et al., 2003, Cell 114: 161-170). Such post-translational modifications are at best is under-represented in aflibercept—a CHO cell product. Unlike human retinal cells, CHO cells are not secretory cells and have a limited capacity for post-translational tyrosine-sulfation. (See, e.g., Mikkelsen & Ezban, 1991, Biochemistry 30: 1533-1537, esp. discussion at p. 1537).
(vii) O-glycosylation comprises the addition of N-acetyl-galactosamine to serine or threonine residues by the enzyme. It has been demonstrated that amino acid residues present in the hinge region of antibodies can be O-glycosylated. In certain embodiments, the VEGF-Trap comprises all or a portion of the IgG Fc hinge region, and thus is capable of being O-glycosylated when expressed in human retinal cells or liver cells. The possibility of O-glycosylation confers another advantage to the VEGF-Trap proteins provided herein, as compared to proteins produced in E. coli, again because E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098).
(viii) In addition to the foregoing post-translational modifications, improved VEGF-Trap constructs can be engineered and used to deliver VEGF-Trap^{HuPTM} to the retina/vitreal humour. For example, because aflibercept has an intact Fc region, it is likely to be salvaged from proteolytic catabolism and recycled via binding to FcRn in endothelial cells; thus prolonging its systemic half-life following entry into the systemic circulation from the eye (e.g., aflibercept has a serum half-life of approximately 4-7 days following intravenous administration). Comparative studies in human subjects receiving 3 monthly intravitreal injections demonstrated that aflibercept and bevacizumab (a full-length antibody) exhibited systemic accumulation after the third dose, whereas ranibizumab (a Fab) did not. (For a review, see Avery et al., 2017, Retina, the Journal of Retinal and Vitreous Diseases 0:1-12; and Avery et al., 2014, Br J Ophthalmol 98:1636-1641). Since prolonged residence of anti-VEGF agents is associated with hemorrhagic and thromboembolic complications, and since aflibercept binds all isoforms of VEGF as well as PLGF, an improved, safer aflibercept can be engineered by modifying the Fc to disable the FcRN binding site or by eliminating the Fc to reduce the half-life of the transgene product following entry into the systemic circulation, yet maintain stability and residence in the eye. Exemplary constructs, designed to eliminate the Fc function yet maintain stability and improve residence in the eye are described herein and illustrated in FIGS. 3 and 4.

For the foregoing reasons, the production of VEGF-Trap^{HuPTM} should result in a “biobetter” molecule for the treatment of nAMD, diabetic retinopathy, DME, cRVO, pathologic myopia, or polypoidal choroidal vasculopathy, accomplished via gene therapy—e.g., by administering a viral vector or other DNA expression construct encoding VEGF-Trap^{HuPTM} to the subretinal space, the suprachoroidal space, or intravitreally in the eye(s) of patients (human subjects) diagnosed with nAMD, diabetic retinopathy, DME, cRVO, pathologic myopia, or polypoidal choroidal vasculopathy, to create a permanent depot in the eye that continuously supplies the fully-human post-translationally modified, e.g., a human-glycosylated, sulfated transgene product (without detectable NeuGC or α-Gal) produced by transduced retinal cells. Retinal cells that may be transduced include but are not limited to retinal neurons; human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells.

In addition, the production of VEGF-Trap^{HuPTM} should result in a “biobetter” molecule for the treatment of cancer, particularly metastatic colon cancer, accomplished via gene therapy—e.g., by administering a viral vector or other DNA expression construct encoding VEGF-Trap^{HuPTM} to the livers of patients (human subjects) diagnosed with cancer, for example by intravenous administration or through the hepatic blood flow, such as by the suprahepatic veins or hepatic artery, particularly metastatic colon cancer, to create a permanent depot in the liver that continuously supplies the fully-human post-translationally modified, e.g., a human-glycosylated, sulfated transgene product (without detectable NeuGC or α-Gal) produced by transduced liver cells.

As an alternative, or an additional treatment to gene therapy, the VEGF-Trap^{HuPTM} glycoprotein can be produced in human cell lines by recombinant DNA technology, and the glycoprotein can be administered to patients diagnosed nAMD, diabetic retinopathy, DME, cRVO, pathologic myopia, or polypoidal choroidal vasculopathy by intravitreal administration or to patients diagnosed with cancer, particularly metastatic colon cancer, by infusion or other parenteral administration. Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Critical Rev in Biotech, 36(6):1110-1122 “Human cell lines for biopharmaceutical manufacturing: history, status, and future perspectives” which is incorporated by reference in its entirety for a review of the human cell lines that could be used for the recombinant production of the VEGF-Trap^{HuPTM} glycoprotein). To ensure complete glycosylation, especially sialylation and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in retinal cells.

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation, including 2,6-sialylation and sulfation to demonstrate efficacy. In certain embodiments, 0.5% to 1% of the population of VEGF-Trap^{HuPTM} has 2,6-sialylation and/or sulfation. In other embodiments, 2%, from 2% to 5%, or 2% to 10% of the population of the VEGF-Trap^{HuPTM} has 2,6-sialylation and/or sulfation. In certain embodiments, the level of 2,6-sialylation and/or sulfation is significantly higher, such that up to 50%, 60%, 70%, 80%, 90% or even 100% of the molecules contain 2,6-sialylation and/or sulfation. The goal of gene therapy treatment provided herein is to treat retinal neovascularization, and to maintain or improve vision with minimal intervention/invasive procedures or to treat, ameliorate or slow the progression of metastatic colon cancer.

Efficacy of treatment for diseases associated with retinal neovascularization may be monitored by measuring BCVA (Best-Corrected Visual Acuity); retinal thickness on SD_OCT (SD-Optical Coherence Tomography) a three-dimensional imaging technology which uses low-coherence interferometry to determine the echo time delay and magnitude of backscattered light reflected off an object of interest (Schuman, 2008, Trans. Am. Opthalmol. Soc. 106:426-458); area of neovascularization on fluorescein angiography (FA); and need for additional anti-VEGF therapy. Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation. Adverse events could include vision loss, ocular infection, inflammation and other safety events, including retinal detachment.

Efficacy of treatment for cancer, particularly metastatic colon cancer, may be monitored by any means known in the art for evaluating the efficacy of an anti-cancer/anti-metastatic agent, such as a reduction in tumor size, reduction in number and/or size of metastases, increase in overall survival, progression free survival, response rate, incidence of stable disease, etc.

Combinations of delivery of the VEGF-Trap^{HuPTM} to the eye/retina accompanied by delivery of other available treatments are described herein. The additional treatments may be administered before, concurrently or subsequent to the gene therapy treatment. Available treatments for nAMD, diabetic retinopathy, DME, cRVO, pathologic myopia, or polypoidal choroidal vasculopathy, that could be combined with the gene therapy of the invention include but are not limited to laser photocoagulation, photodynamic therapy with verteporfin, and intravitreal (IVT) injections with anti-VEGF agents, including but not limited to aflibercept, ranibizumab, bevacizumab, or pegaptanib, as well as treatment with intravitreal steroids to reduce inflammation. Available treatments for metastatic colon cancer, that could be combined with the gene therapy of the invention include but are not limited to 5-fluorouracil, leucovorin, irinotecan (FOLFIRI) or folinic acid (also called leucovorin, FA or calcium folinate), fluorouracil (5FU), and/or oxaliplatin (FOLFOX), and intravenous administration with anti-VEGF agents, including but not limited to ziv-aflibercept, ranibizumab, bevacizumab, pegaptanib or regorafenib.

Provided also are methods of manufacturing the AAV8 viral vectors containing the VEGF-Trap transgenes and the VEGF-Trap^{HuPTM} protein products. In specific embodiments, methods are provided for making AAV8 viral vectors containing the VEGF-Trap transgene by culturing host cells that are stably transformed with a nucleic acid vector comprising an expression cassette flanked by AAV inverted terminal repeats (ITRs) wherein the expression cassette comprises a transgene encoding a VEGF-Trap^{HuPTM}, operably linked to one or more regulatory sequences that control expression of the transgene in human retinal cells or human liver cells and also comprise nucleotide sequences encoding the AAV8 replication and capsid proteins and recovering the AAV8 viral vector produced by the host cell.

The invention is illustrated in the examples, infra, describe VEGF-Trap^{HuPTM} constructs packaged in AAV8 capsid for subretinal injection or intravenous administration in human subjects.

### 3.1. Illustrative Embodiments

1. An expression construct comprising an expression cassette flanked by AAV inverted terminal repeats (ITRs) wherein the expression cassette comprises a transgene encoding a VEGF-Trap^{HuPTM}, operably linked to one or more regulatory sequences that control expression of the transgene in human retinal cells or in human liver cells.

2. The expression construct of paragraph 1 wherein the transgene encodes a VEGF-Trap^{HuPTM} having the amino acid sequence set forth in FIG. 1, FIG. 2, FIG. 3, FIG. 4, FIGS. 7C-7H, or FIGS. 8C-8D.

3. The expression construct of paragraph 1 or 2, wherein the transgene comprises a leader sequence at its N-terminus of Table 3 or 4.

4. The expression construct of any of paragraphs 1 to 3, wherein the transgene comprises the nucleotide sequence of SEQ ID NO: 2 or 3 encoding the VEGF-Trap^{HuPTM}.

5. The expression construct of any of paragraphs 1 to 4 wherein at least one of the regulatory sequences is a constitutive promoter.

6. The expression construct of any of paragraphs 1 to 5 wherein the one or more regulatory sequences are a CB7 promoter, a chicken β-actin intron and a rabbit β-globin poly A signal.

7. The expression construct of any of paragraphs 1 to 4 wherein at least one of the regulatory sequences is an inducible promoter.

8. The expression construct of paragraph 7 wherein the inducible promoter is a hypoxia-inducible promoter or a rapamycin inducible promoter.

9. The expression construct of any of paragraphs 1 to 8, wherein the AAV ITRs are AAV2 ITRs.

10. The expression construct of any of paragraphs 1 to 6 or 9, which is the expression construct of one of FIGS. 5A-5E.

11. An adeno-associated virus (AAV) vector comprising a viral capsid that is at least 95% identical to the amino acid sequence of an AAV8 capsid (SEQ ID NO: 11); and a viral genome comprising an expression cassette flanked by AAV ITRs wherein the expression cassette comprises a transgene encoding a VEGF-Trap^{HuPTM}, operably linked to one or more regulatory sequences that control expression of the transgene in human retinal cells or in human liver cells.

12. The AAV vector of paragraph 11 wherein the transgene encodes a VEGF-Trap^{HuPTM} having the amino acid sequence set forth in FIG. 1, FIG. 2, FIG. 3, FIG. 4, FIGS. 7C-7H, or FIGS. 8C-8D.

13. The AAV vector of paragraph 11 or 12, wherein the transgene comprises a leader sequence at its N-terminus of Table 3 or 4.

14. The AAV vector of any of paragraphs 11 to 13, which comprises the nucleotide sequence of SEQ ID NO: 2 or 3 encoding the VEGF-Trap^{HuPTM}.

15. The AAV vector of any of paragraphs 11 to 14 wherein at least one of the regulatory sequences is a constitutive promoter.

16. The AAV vector of any of paragraphs 11 to 15 wherein the one or more regulatory sequences are a CB7 promoter, a chicken β-actin intron and a rabbit β-globin poly A signal.

17. The AAV vector of any of paragraphs 11 to 14 wherein at least one of the regulatory sequences is an inducible promoter.

18. The AAV vector of paragraph 17 wherein the inducible promoter is a hypoxia-inducible promoter or a rapamycin inducible promoter.

19. The AAV vector of any of paragraphs 11 to 18, wherein the AAV ITRs are AAV2 ITRs.

20. A pharmaceutical composition for treating ocular disorders, including age-related macular degeneration, in a human subject in need thereof, comprising an AAV vector comprising:
a viral capsid that is at least 95% identical to the amino acid sequence of an AAV8 capsid (SEQ ID NO: 11); and
a viral genome comprising an expression cassette flanked by AAV ITRs wherein the expression cassette comprises a transgene encoding a VEGF-Trap, operably linked to one or more regulatory sequences that control expression of the transgene in human retinal cells;
wherein said AAV vector is formulated for subretinal, intravitreal or suprachoroidal administration to the eye of said subject.

21. A pharmaceutical composition for treating ocular disorders, including age-related macular degeneration, in a human subject in need thereof, comprising an adeno-associated virus (AAV) vector comprising:
a viral capsid that is at least 95% identical to the amino acid sequence of an AAV8 capsid (SEQ ID NO: 11); and
a viral genome comprising an expression cassette flanked by AAV ITRs wherein the expression cassette comprises a transgene encoding a VEGF-Trap, operably linked to one or more regulatory sequences that control expression of the transgene in human liver cells;
wherein said AAV vector is formulated for intravenous administration to said subject.

22. A pharmaceutical composition for treating ocular disorders, including age-related macular degeneration, in a human subject in need thereof, comprising an adeno-associated virus (AAV) vector comprising:
a viral capsid that is at least 95% identical to the amino acid sequence of an AAV.7m8 capsid; and
a viral genome comprising an expression cassette flanked by AAV ITRs wherein the expression cassette comprises a transgene encoding a VEGF-Trap, operably linked to one or more regulatory sequences that control expression of the transgene in human liver cells;
wherein said AAV vector is formulated for intravenous administration to said subject.

23. The pharmaceutical composition of paragraphs 20 to22, wherein the VEGF-Trap has the amino acid sequence set forth in FIG. 1, FIG. 2, FIG. 3, FIG. 4, FIGS. 7C-7H, or FIGS. 8C-8D.

24. The pharmaceutical composition of any of paragraphs 20 to 23, wherein the transgene comprises a leader sequence at its N-terminus of Table 3 or 4.

25. The pharmaceutical composition of any of paragraphs 20 to 24, wherein the transgene comprises the nucleotide sequence of SEQ ID NO: 2 or 3 encoding the VEGF-Trap^{HuPTM}.

26. The pharmaceutical composition of any of paragraphs 20 to 25 wherein at least one of the regulatory sequences is a constitutive promoter.

27. The pharmaceutical composition of any of paragraphs 20 to 26 wherein the one or more regulatory sequences are a CB7 promoter, a chicken β-actin intron and a rabbit β-globin poly A signal.

28. The pharmaceutical composition of any of paragraphs 20 to 25 wherein at least one of the regulatory sequences is an inducible promoter.

29. The pharmaceutical composition of paragraph 28 wherein the inducible promoter is a hypoxia-inducible promoter or a rapamycin inducible promoter.

30. The pharmaceutical composition of any of paragraphs 20 to 29, wherein the AAV ITRs are AAV2 ITRs.

31. A method of treating a human subject diagnosed with neovascular age-related macular degeneration (nAMD), diabetic retinopathy, diabetic macular edema (DME), central retinal vein occlusion (RVO), pathologic myopia, or polypoidal choroidal vasculopathy, said method comprising delivering to the retina of said human subject therapeutically effective amount of VEGF-Trap^{HuPTM} produced by human retinal cells.

32. A method of treating a human subject diagnosed with nAMD, diabetic retinopathy, DME, RVO, pathologic myopia, or polypoidal choroidal vasculopathy, said method comprising delivering to the retina of said human subject therapeutically effective amount of VEGF-Trap^{HuPTM} produced by human retinal neurons, human photoreceptor cells, human cone cells, human rod cells, human horizontal cells, human bipolar cells, human amarcrine cells, human retina ganglion cells, human midget cells, human parasol cells, human bistratified cells, human giant retina ganglion cells, human photosensitive ganglion cells, human muller glia, or human retinal pigment epithelial cells.

33. A method of treating a human subject diagnosed with metastatic colon cancer, said method comprising delivering to the colon cancer cells and/or tissue surrounding said colon cancer cells of said human subject therapeutically effective amount of VEGF-Trap^{HuPTM} produced by human liver cells.

34. The method of any of paragraphs 31 to 33 in which the VEGF-Trap^{HuPTM} has the amino acid sequence of SEQ ID NO:1.

35. The method of any of paragraphs 31 to 34 in which the VEGF-Trap^{HuPTM} is a variant of the amino acid sequence of SEQ ID NO:1 with a disabled FcRn binding site.

36. The method of paragraph 35 in which the VEGF-Trap^{HuPTM} has an amino acid substitution of alanine or glutamine for histidine at position 420 of SEQ ID NO:1.

37. The method of paragraph 35 in which the VEGF-Trap^{HuPTM} has the IgG1 Fc domain deleted from SEQ ID NO:1.

38. The method of paragraph 35 in which the IgG1 Fc domain of SEQ ID NO:1 is substituted with an IgG2 Fc domain, and IgG4 Fc domain, one or more IgG-like domains of human Flt-1, or one or more IgG-like domains of human KDR, or a combination of one or more IgG-like domains of human Flt-1 and IgG-like domains of human KDR.

39. The method of paragraph 35 in which the VEGF-Trap^{HuPTM} has the amino acid sequence set forth in one of FIG. 2, FIG. 3, FIG. 4, FIGS. 7C-7H, or FIGS. 8C-8D.

40. The method of any of paragraphs 31 to 39, wherein the VEGF-Trap^{HuPTM} comprises a leader sequence at its N-terminus of Table 3 or 4.

41. A method of treating a human subject diagnosed with nAMD, diabetic retinopathy, DME, RVO, pathologic myopia, or polypoidal choroidal vasculopathy, said method comprising delivering to the retina of the eye of said human subject, a therapeutically effective amount of a VEGF-Trap^{HuPTM} containing a α2,6-sialylated glycan.

42. A method of treating a human subject diagnosed with nAMD, diabetic retinopathy, DME, RVO, pathologic myopia, or polypoidal choroidal vasculopathy, said method comprising delivering to the retina of the eye of said human subject, a therapeutically effective amount of a VEGF-Trap^{HuPTM} containing a tyrosine-sulfation.

43. A method of treating a human subject diagnosed with metastatic colon cancer, said method comprising delivering to the colon cancer cells and/or tissue surrounding said colon cancer cells of said human subject, a therapeutically effective amount of a VEGF-Trap^{HuPTM} containing a α2,6-sialylated glycan.

44. A method of treating a human subject diagnosed with metastatic colon cancer, said method comprising delivering to the colon cancer cells and/or tissue surrounding said colon cancer cells of said human subject, a therapeutically effective amount of a VEGF-Trap^{HuPTM} containing a tyrosine-sulfation.

45. The method of any of paragraphs 41 to 44 wherein the VEGF-Trap^{HuPTM} does not contain detectable NeuGc or α-Gal.

46. The method of any of paragraphs 41 to 45 wherein the VEGF-Trap^{HuPTM} contains a α2,6-sialylated glycan and a tyrosine sulfation and does not contain detectable NeuGc or α-Gal.

47. The method of any of paragraphs 41 to 46 in which the VEGF-Trap^{HuPTM} has the amino acid sequence set forth in one of FIG. 1, FIG. 2, FIG. 3, FIG. 4, FIGS. 7C-7H, or FIGS. 8C-8D.

48. A method of treating a human subject diagnosed with nAMD, diabetic retinopathy, DME, RVO, pathologic myopia, or polypoidal choroidal vasculopathy, said method comprising: administering to the subretinal space in the eye of said human subject, a therapeutically effective amount of a recombinant nucleotide expression vector encoding a VEGF-Trap^{HuPTM} so that a depot is formed that releases said VEGF-Trap^{HuPTM} containing a α2,6-sialylated glycan.

49. A method of treating a human subject diagnosed with nAMD, diabetic retinopathy, DME, RVO, pathologic myopia, or polypoidal choroidal vasculopathy, comprising: administering to the subretinal space in the eye of said human subject, a therapeutically effective amount of a recombinant nucleotide expression vector encoding a VEGF-Trap^{HuPTM} so that a depot is formed that releases said VEGF-Trap^{HuPTM} containing a tyrosine-sulfation.

50. A method of treating a human subject diagnosed with metastatic colon cancer, said method comprising: administering to the liver of said human subject, a therapeutically effective amount of a recombinant nucleotide expression vector encoding a VEGF-Trap^{HuPTM} so that a depot is formed that releases said VEGF-Trap^{HuPTM} containing a α2,6-sialylated glycan.

51. A method of treating a human subject diagnosed with metastatic colon cancer, said method comprising: administering to the liver of said human subject, a therapeutically effective amount of a recombinant nucleotide expression vector encoding a VEGF-Trap^{HuPTM} so that a depot is formed that releases said VEGF-Trap^{HuPTM} containing a tyrosine-sulfation.

52. The method of any of paragraphs 48 or 51 wherein the VEGF-Trap^{HuPTM} does not contain detectable NeuGc or α-Gal.

53. The method of any of paragraphs 48 to 52 wherein the VEGF-Trap^{HuPTM} contains a α2,6-sialylated glycan and a tyrosine sulfation and does not contain any detectable NeuGc or α-Gal.

54. The method of any of paragraphs 48 to 53 in which the VEGF-Trap^{HuPTM} has the amino acid sequence set forth in one of FIG. 1, FIG. 2, FIG. 3, FIG. 4, FIGS. 7C-7H, or FIGS. 8C-8D.

55. The method of any of paragraphs 48 to 54, wherein the recombinant nucleotide expression vector comprises a nucleotide sequence of SEQ ID NO: 2 or 3 that encodes the VEGF-Trap^{HuPTM}.

56. The method of any of paragraphs 48 to 55 wherein the recombinant nucleotide expression vector is an AAV8 viral vector.

57. The method of any of paragraphs 48 to 55 wherein the recombinant nucleotide expression vector is an AAV.7m8 viral vector.

58. The method of any of paragraphs claim 41, 43, 45-48, 50, or 52-57 in which production of said VEGF-Trap^{HuPTM} containing a α2,6-sialylated glycan is confirmed by transducing PER.C6 or RPE cell line with said recombinant nucleotide expression vector in cell culture.

59. The method of any of paragraphs 42, 44-47, 49, or 51-57 in which production of said VEGF-Trap^{HuPTM} containing a tyrosine-sulfation is confirmed by transducing PER.C6 or RPE cell line with said recombinant nucleotide expression vector in cell culture.

60. A method of producing recombinant AAVs comprising:
(a) culturing a host cell containing:
   (i) an artificial genome comprising a cis expression cassette flanked by AAV ITRs, wherein the cis expression cassette comprises a transgene encoding a VEGF-Trap operably linked to expression control elements that will control expression of the transgene in retinal cells or liver cells;
   (ii) a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans;
   (iii) sufficient adenovirus helper functions to permit replication and packaging of the artificial genome by the AAV capsid proteins; and
(b) recovering recombinant AAV encapsidating the artificial genome from the cell culture.

61. A method of manufacturing an AAV8 viral vector comprising a VEGF-Trap transgene, said method comprising culturing host cells that are stably transformed with a nucleic acid vector comprising an expression cassette flanked by AAV ITRs wherein the expression cassette comprises a transgene encoding a VEGF-Trap^{HuPTM}, operably linked to one or more regulatory sequences that control expression of the transgene in human retinal cells and also comprise nucleotide sequences encoding the AAV8 replication and capsid proteins under conditions appropriate for production of the AAV8 viral vector; and recovering the AAV8 viral vector produced by the host cell.

62. A method of manufacturing a VEGF-Trap^{HuPTM}, said method comprising culturing an immortalized human retinal cell transformed with an expression vector a nucleotide sequence encoding the VEGF-Trap^{HuPTM}, operably linked to one or more regulatory sequences that control expression of the VEGF-Trap^{HuPTM} in human retinal cells and isolating the VEGF-Trap^{HuPTM} expressed by the human retinal cells.

### 4. BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. The amino acid sequence of the fusion protein of aflibercept, including the leader sequence that is at the N-terminal of the protein (SEQ ID NO: 15). The leader sequence is not numbered. N-linked glycosylation sites are highlighted in yellow at positions 36, 68, 123, 196 and 282; tyrosine-O-sulfation sites are highlighted in red at positions 11, 140, 263, and 281; cysteines involved in disulfide bonding are highlighted in green at positions 30, 79, 124, 185, 211, 214, 246, 306, 352, and 410; and Fc domain positions that may be substituted to reduce FcRn binding are highlighted in pink at positions 238, 295, and 420. The Flt-1 sequence is in orange text (the Ig-like Domain 2 in bold) from positions 1 to 102, the KDR sequence is in blue text (the Ig-like Domain 3 in bold) from positions 103 to 205, and the IgG1 Fc is in gray from position 206, with the hinge region indicated in italics.

FIG. 2. The amino acid sequence of the fusion protein of aflibercept with a heterologous signal peptide (SEQ ID NO: 16). N-linked glycosylation sites are highlighted in yellow at positions 36, 68, 123, 196 and 282; tyrosine-O-sulfation sites highlighted in red at positions 11, 140, 263, and 281; cysteines involved in disulfide bonding are highlighted in green at positions 30, 79, 124, 185, 211, 214, 246, 306, 352, and 410; and Fc domain positions that may be substituted to reduce FcRn binding are highlighted in pink at positions 238, 295, and 420. The Flt-1 sequence is in orange text (the Ig-like Domain 2 in bold) from positions 1 to 102, the KDR sequence is in blue text (the Ig-like Domain 3 in bold) from positions 103 to 205, and the IgG1 Fc is in gray from position 206, with the hinge region indicated in italics.

FIG. 3. The amino acid sequence of the fusion protein of aflibercept H420A/Q (disabled Fc) with a heterologous signal peptide (SEQ ID NO: 17). N-linked glycosylation sites are highlighted in yellow at positions 36, 68, 123, 196 and 282; tyrosine-O-sulfation sites highlighted in red at positions 11, 140, 263, and 281; cysteines involved in disulfide bonding are highlighted in green at positions 30, 79, 124, 185, 211, 214, 246, 306, 352, and 410. The Flt-1 sequence is in orange text (the Ig-like Domain 2 in bold) from positions 1 to 102, the KDR sequence is in blue text (the Ig-like Domain 3 in bold) from positions 103 to 205, and the IgG1 Fc is in gray from position 206, with the hinge region indicated in italics.

FIG. 4. The amino acid sequence of the fusion protein of aflibercept.Fc⁽⁻⁾ with a heterologous signal peptide (SEQ ID NO: 18). N-linked glycosylation sites are highlighted in yellow at positions 36, 68, 123, and 196; tyrosine-O-sulfation sites highlighted in red at positions 11 and 140; cysteines involved in disulfide bonding are highlighted in green at positions 30, 79, 124 and 185, (optionally 211 and 214). The Flt-1 sequence is in orange text (the Ig-like Domain 2 in bold) from positions 1 to 102, and the KDR sequence is in blue text (the Ig-like Domain 3 in bold) from positions 103 to 205. Fc-less variants are indicated in gray and may include K, KDKTHT (SEQ ID NO: 31) (or KDKTHL (SEQ ID NO: 32)), KDKTHTCPPCPA (SEQ ID NO: 33) or KDKTHTCPPCPAPELLGG (SEQ ID NO: 34), or KDKTHTCPPCPAPELLGGPSVFL (SEQ ID NO: 35).

FIGS. 5A-5F. VEGF-Trap constructs. (A) is an AAV8 expression construct for expression of the fusion protein with the amino acid sequence of aflibercept, as set forth in FIG. 1; (B) is an AAV8 expression construct for expression of the fusion protein with the amino acid sequence of aflibercept having an alternate leader sequence, as set forth in FIG. 2; (C) is an AAV8 expression construct for expression of the fusion protein with the amino acid sequence of aflibercept with an H420A (“H435A”) substitution and an alternate leader sequence, as set forth in FIG. 3 (with the substitution at position 420 as numbered in FIG. 3); (D) is an AAV8 expression construct for expression of the fusion protein with the amino acid sequence of aflibercept with an H420Q (“H435Q”) substitution and an alternate leader sequence, as set forth in FIG. 3 (with the substitution at position 420 as numbered in FIG. 3); (E) is an AAV8 expression construct that is bicistronic for expression of two copies of the Fc-less VEGF-Trap^{HuPTM} having an IRES between the two copies of nucleotide sequence encoding the Fc-less VEGF-Trap^{HuPTM}; and (F) is an AAV8 expression construct for expression of two copies of the Fc-less VEGF-Trap^{HuPTM} with a cleavable furin/furin 2A linker and an alternate leader sequence.

FIG. 6. Clustal Multiple Sequence Alignment of AAV capsids 1-9. The last row “SUBS” indicates amino acid substitutions that may be made (shown in bold in the bottom rows) can be made to the AAV8 capsid by “recruiting” amino acid residues from the corresponding position of other aligned AAV capsids. The hypervariable regions are shown in red. The amino acid sequences of the AAV capsids are assigned SEQ ID NOs as follows: AAV1 is SEQ ID NO: 4; AAV2 is SEQ ID NO: 5; AAV3-3 is SEQ ID NO: 6; AAV4-4 is SEQ ID NO: 7; AAVS is SEQ ID NO: 8; AAV6 is SEQ ID NO: 9; AAV7 is SEQ ID NO: 10; AAV8 is SEQ ID NO: 11; hu31 is SEQ ID NO: 12; hu32 is SEQ ID NO: 13; and AAV9 is SEQ ID NO: 14.

FIGS. 7A-H. The amino acid sequences of (A) Fc domain of IgG2, with the hinge region in italics and underline (SEQ ID NO: 19); (B) the Fc domain of IgG4, with the hinge region in italics and underline (SEQ ID NO: 20); (C) VEGF-Trap^{HuPTM} with an IgG2 Fc domain with a partial hinge region as the C-terminal domain (SEQ ID NO: 21); (D) VEGF-Trap^{HuPTM} having an IgG2 Fc with a full hinge region as the C-terminal domain (SEQ ID NO: 22); (E) VEGF-Trap^{HuPTM} having an IgG4 Fc with a partial hinge region as the C-terminal domain(SEQ ID NO: 23); (F) VEGF-Trap^{HuPTM} having an IgG4 Fc with a partial hinge region as the C-terminal domain in which two cysteine residues are substituted with serine residues at underlined positions (SEQ ID NO: 24); (G) VEGF-Trap^{HuPTM} having a IgG4 Fc with a full hinge region as the C-terminal domain (SEQ ID NO: 25); and (H) VEGF-Trap^{HuPTM} having an IgG4 Fc with a full hinge region as the C-terminal domain in which two cysteine residues are substituted with serine at the underlined position (SEQ ID NO: 26). In C through H, the Flt 1 sequence is in orange text from positions 1 to 102 and the KDR sequence is in blue text from positions 103 to 205.

FIGS. 8A-D. The amino acid sequences of (A) the extracellular domain and signal sequence of human Flt-1 (UniProtKB—P17948 (VGFR1_HUMAN)), with the signal sequence italicized, Ig-like domain 1 sequence in blue, the Ig-like domain s sequence in green, the Ig-like domain 3 sequence in orange, the Ig-like domain 4 sequence in red, the Ig-like domain 5 sequence in yellow, the Ig-like domain 6 in purple, and the Ig-like domain 7 in gray (SEQ ID NO: 27); (B) the extracellular domain and signal sequence of human KDR (UniProtKB P35968 (VGFR2_HUMAN)), with the signal sequence italicized, the Ig-like domain 1 sequence in blue, the Ig-like domain 2 sequence in green, the Ig-like domain 3 sequence in orange, the Ig-like domain type 4 sequence in red, the Ig-like domain 5 sequence in yellow, the Ig-like domain 6 in purple, and the Ig-like domain 7 in gray (SEQ ID NO: 28); (C) a VEGF-Trap^{HuPTM} with Flt-1 Ig-like domains as the C terminal domain (SEQ ID NO: 29); and (D) a VEGF-Trap^{HuPTM} with KDR Ig-like domains as the C terminal domain (SEQ ID NO: 30). For both 8C and 8D, the the Ig-like domain 2 of Flt 1 sequence is in orange text from positions 1 to 102 and the the Ig-like domain 3 of KDR sequence is in blue text from positions 103 to 205.

### DETAILED DESCRIPTION OF THE INVENTION

Compositions and methods are provided for the delivery of a human-post-translationally modified VEGF-Trap (VEGF-Trap^{HuPTM}) to the retina/vitreal humour in the eye(s) of patients (human subjects) diagnosed with an ocular disease caused by increased vascularization, for example, nAMD, also known as “wet” AMD. This may be accomplished via gene therapy—e.g., by administering a viral vector or other DNA expression construct encoding (as a transgene) a VEGF-Trap protein to the eye(s) of patients (human subjects) diagnosed with nAMD, or other ocular disease caused by vascularization, to create a permanent depot in the eye that continuously supplies the fully human post-translationally modified transgene product. Such DNA vectors can be administered to the subretinal space, or to the suprachoroidal space, or intravitreally to the patient. The VEGF-Trap^{HuPTM} may have fully human post-translational modifications due to expression in human cells (as compared to non-human CHO cells). The method can be used to treat any ocular indication that responds to VEGF inhibition, especially those that respond to aflibercept (EYLEA®): e.g., AMD, diabetic retinopathy, diabetic macular edema (DME), including diabetic retinopathy in patients with DME, central retinal vein occlusion (RVO) and macular edema following RVO, pathologic myopia, particularly as caused by myopic choroidal neovascularization, and polypoidal choroidal vasculopathy, to name a few.

In other embodiments, provided are compositions and methods for delivery of a VEGF-Trap^{HuPTM} to cancer cells and surrounding tissue, particularly tissue exhibiting increased vascularization, in patients diagnosed with cancer, for example, metastatic colon cancer. This may be accomplished via gene therapy—e.g., by administering a viral vector or other DNA expression construct encoding as a transgene a VEGF-Trap protein to the liver of patients (human subjects) diagnosed with cancer, particularly metastatic colon cancer, to create a permanent depot in the liver that continuously supplies the fully human post-translationally modified transgene product. Such DNA vectors can be administered intravenously to the patient or directly to the liver through hepatic blood flow, e.g., via the suprahepatic veins or via the hepatic artery.

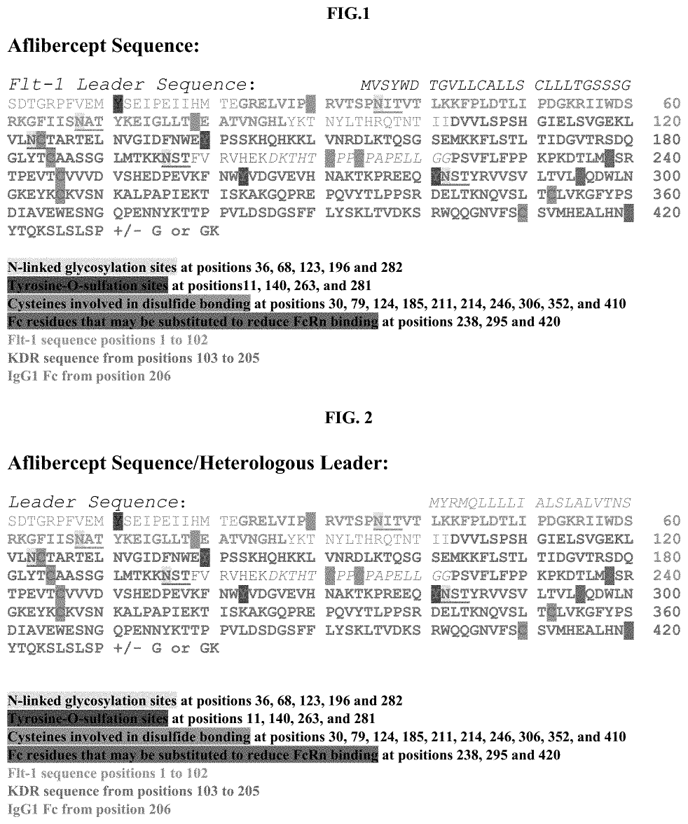

The VEGF-Trap^{HuPTM} encoded by the transgene is a fusion protein which comprises (from amino to carboxy terminus): (i) the Ig-like domain 2 of Flt-1 (human; also named VEGFR1), (ii) the Ig-like domain 3 of KDR (human; also named VEGFR2), and (iii) a human IgG Fc region, particularly a IgG1 Fc region. In specific embodiments, the VEGF-Trap^{HuPTM} has the amino acid sequence of aflibercept (SEQ ID NO: 1 and FIG. 1, which provide the numbering of the amino acid positions in FIG. 1 will be used herein; see also Table 1, infra for amino acid sequence of aflibercept and codon optimized nucleotide sequences encoding aflibercept). FIG. 1 also provides the Flt-1 leader sequence at the N-terminus of the aflibercept sequence, and the transgene may include the sequence coding for the leader sequence of FIG. 1 or other alternate leader sequences as disclosed infra. Alternatively, the transgene may encode variants of a VEGF-Trap designed to increase stability and residence in the eye, yet reduce the systemic half-life of the transgene product following entry into the systemic circulation; truncated or “Fc-less” VEGF-Trap constructs, VEGF Trap transgenes with a modified Fc, wherein the modification disables the FcRn binding site and or where another Fc region or Ig-like domain is substituted for the IgG1 Fc domain.

In certain aspects, provided herein are constructs for the expression of VEGF-Trap transgenes in human retinal or liver cells. The constructs can include expression vectors comprising nucleotide sequences encoding a transgene and appropriate expression control elements for expression in retinal or liver cells. The recombinant vector used for delivering the transgene should have a tropism for retinal or liver cells. These can include non-replicating recombinant adeno-associated virus vectors (“rAAV”), particularly those bearing an AAV8 capsid, or variants of an AAV8 capsid are preferred. However, other viral vectors may be used, including but not limited to lentiviral vectors, vaccinia viral vectors, or non-viral expression vectors referred to as “naked DNA” constructs.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161). Provided as SEQ ID NO: 2 is a codon optimized nucleotide sequence that encodes the transgene product of SEQ ID NO: 1, plus the leader sequence provided in FIG. 1. SEQ ID NO: 3 is a consensus codon optimized nucleotide sequence encoding the transgene product of SEQ ID NO: 1 plus the leader sequence in FIG. 1 (see Table 1, infra, for SEQ ID NOs: 2 and 3).

In specific embodiments, provided are constructs for gene therapy administration for treating ocular disorders, including macular degeneration (nAMD), diabetic retinopathy, diabetic macular edema (DME), central retinal vein occlusion (RVO), pathologic myopia, or polypoidal choroidal vasculopathy, in a human subject in need thereof, comprising an AAV vector, which comprises a viral capsid that is at least 95% identical to the amino acid sequence of an AAV8 capsid (SEQ ID NO: 11); and a viral genome comprising an expression cassette flanked by AAV inverted terminal repeats (ITRs) wherein the expression cassette comprises a transgene encoding a VEGF-Trap^{HuPTM}, operably linked to one or more regulatory sequences that control expression of the transgene in human retinal cells.

The construct for the VEGF-Trap^{HuPTM} should include a nucleotide sequence encoding a signal peptide that ensures proper co- and post-translational processing (glycosylation and protein sulfation) by the transduced retinal cells or liver cells. In preferred embodiments, the signal sequence is that of Flt-1, MVSYWDTGVLLCALLSCLLLTGSSSG (SEQ ID NO: 36) (see FIG. 1). In alternative embodiments, the signal sequence is the KDR signal sequence, MQSKVLLAVALWLCVETRA (SEQ ID NO: 37), or alternatively, in preferred embodiments, MYRMQLLLLIALSLALVTNS (SEQ ID NO: 38) or MRMQLLLLIALSLALVTNS (SEQ ID NO: 39) (see FIG. 2). Other signal sequences used for expression in human retinal cells may include, but are not limited to, those in Table 3, infra, and signal sequences used for expression in human liver cells may include, but are not limited to, those in Table 4 infra.

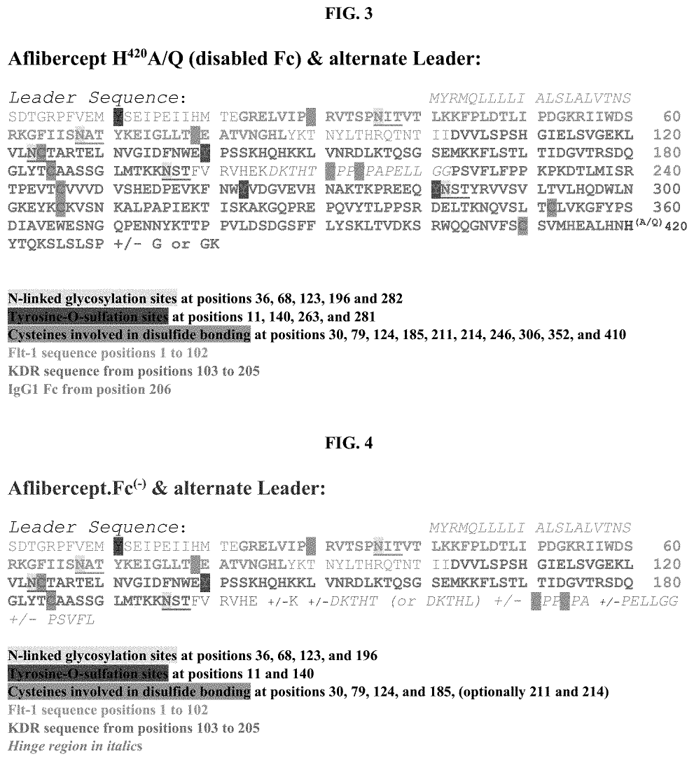

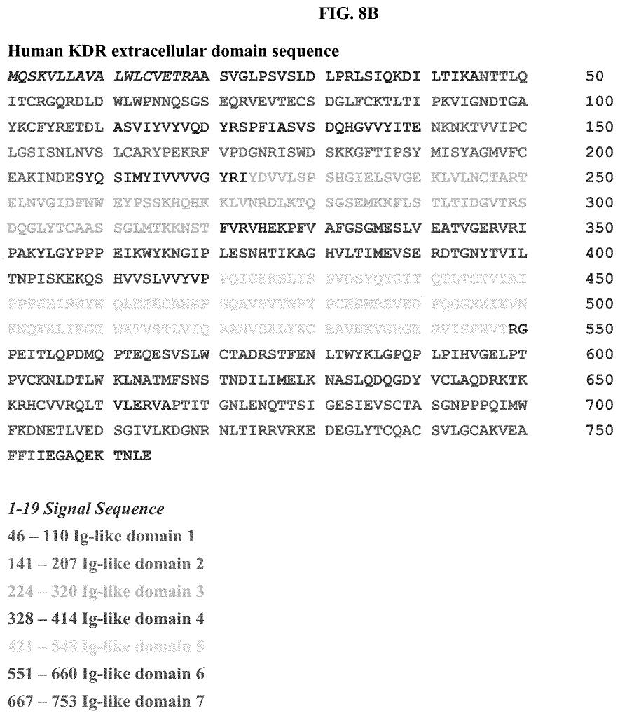

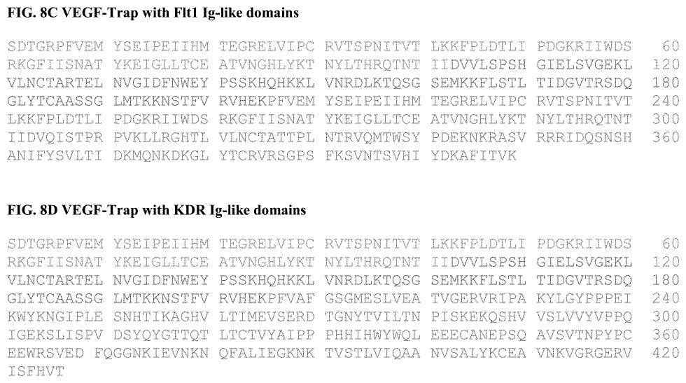

In specific embodiments, the VEGF-Trap^{HuPTM} has the amino acid sequence set forth in FIG. 1, FIG. 2, FIG. 3, FIG. 4, FIGS. 7C-7H or FIGS. 8C and 8D.

In certain aspects, described herein are methods of treating a human subject diagnosed with neovascular age-related macular degeneration (nAMD), diabetic retinopathy, diabetic macular edema (DME), central retinal vein occlusion (RVO), pathologic myopia, or polypoidal choroidal vasculopathy, comprising delivering to the retina of said human subject a therapeutically effective amount of a VEGF-Trap^{HuPTM} produced by human retinal cells, including human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells. In certain embodiments, the VEGF-Trap^{HuPTM} is delivered by administering to the eye of the patient a therapeutically effective amount of a recombinant nucleotide expression vector encoding a VEGF-Trap^{HuPTM}, so that a depot is formed in retinal cells that releases said VEGF-Trap^{HuPTM} which is then delivered to the retina.

In certain aspects, described herein are methods of treating a human subject diagnosed with cancer, particularly metastatic colon cancer, comprising delivering to the cancer cells or surrounding tissue (e.g., the tissue exhibiting increased vascularization surrounding the cancer cells) of said human subject a therapeutically effective amount of a VEGF-Trap^{HuPTM} produced by human liver cells. In certain embodiments, the VEGF-Trap^{HuPTM} is delivered by administering a therapeutically effective amount of a recombinant nucleotide expression vector encoding a VEGF-Trap^{HuPTM} to a patient diagnosed with cancer, preferably intravenously, so that a depot is formed in the liver that releases said VEGF-TrapHuPTM which is then delivered to the cancer cells and/or surrounding tissue.

Subjects to whom such gene therapy is administered should be those responsive to anti-VEGF therapy. In particular embodiments, the methods encompass treating patients who have been diagnosed with nAMD, diabetic retinopathy, DME, cRVO, pathologic myopia, or polypoidal choroidal vasculopathy, or diagnosed with cancer, and identified as responsive to treatment with a VEGF-Trap protein or other anti-VEGF agent.

In certain aspects, provided herein are VEGF-Trap proteins that contain human post-translational modifications. In one aspect, the VEGF-Trap proteins described herein contains the human post-translational modification of α2,6-sialylated glycans. In certain embodiments, the VEGF-Trap proteins only contain human post-translational modifications. In one embodiment, the VEGF-Trap proteins described herein do not contain the immunogenic non-human post-translational modifications of Neu5Gc and/or α-Gal. In another aspect, the VEGF-Trap proteins contain tyrosine (“Y”) sulfation sites. In one embodiment the tyrosine sites are sulfated in the Flt-1 Ig-like domain 2, the KDR Ig-like domain 3, and/or Fc domain of aflibercept (see FIG. 1 for sulfation sites, highlighted in red). In another aspect, the VEGF-Trap proteins contain α2,6-sialylated glycans and at least one sulfated tyrosine site. In other aspects, the VEGF-Trap proteins contain fully human post-translational modifications (VEGF-Trap^{HuPTM}). In certain aspects, the post-translational modifications of the VEGF-Trap can be assessed by transducing PER.C6 or RPE cells in culture with the transgene, which can result in production of said VEGF-Trap that has 2,6-sialylation but does not contain detectable (as determined by standard assays, e.g., as described infra) NeuGc or α-Gal in the cell culture. Alternatively, or in addition, the production of said VEGF-Trap containing a tyrosine-sulfation can confirmed by transducing PER.C6 or RPE cell line with said recombinant nucleotide expression vector in cell culture.

The invention has several advantages over standard of care treatments that involve repeated ocular injections of high dose boluses of the VEGF inhibitor that dissipate over time resulting in peak and trough levels. Sustained expression of the transgene product VEGF-Trap, as opposed to injecting a VEGF-Trap product repeatedly, allows for a more consistent levels of the therapeutic to be present at the site of action, and is less risky and more convenient for patients, since fewer injections need to be made, resulting in fewer doctor visits. Furthermore, VEGF-Traps expressed from transgenes are post-translationally modified in a different manner than those that are directly injected because of the different microenvironment present during and after translation. Without being bound by any particular theory, this results in VEGF-Trap molecules that have different diffusion, bioactivity, distribution, affinity, pharmacokinetic, and immunogenicity characteristics, such that the antibodies delivered to the site of action are “biobetters” in comparison with directly injected VEGF-Traps.

The production of VEGF-Trap^{HuPTM} should result in a “biobetter” molecule for the treatment of nAMD, diabetic retinopathy, DME, cRVO, pathologic myopia, or polypoidal choroidal vasculopathy, accomplished via gene therapy—e.g., by administering a viral vector or other DNA expression construct encoding VEGF-Trap^{HuPTM} to the subretinal space, the suprachoroidal space, or intravitreally in the eye(s) of patients (human subjects) diagnosed with nAMD, diabetic retinopathy, DME, cRVO, pathologic myopia, or polypoidal choroidal vasculopathy, to create a permanent depot in the eye that continuously supplies the fully-human post-translationally modified, e.g., a human-2,6-sialylated, sulfated transgene product (without detectable NeuGC or α-Gal) produced by transduced retinal cells. In addition, the production of VEGF-Trap^{HuPTM} should result in a “biobetter” molecule for the treatment of cancer, particularly metastatic colon cancer, accomplished via gene therapy—e.g., by administering a viral vector or other DNA expression construct encoding VEGF-Trap^{HuPTM} to the livers of patients (human subjects) diagnosed with cancer, particularly metastatic colon cancer, to create a permanent depot in the liver that continuously supplies the fully-human post-translationally modified, e.g., a human-2,6 sialylated, sulfated transgene product (without detectable NeuGC or α-Gal) produced by transduced liver cells.

As an alternative, or an additional treatment to gene therapy, the VEGF-Trap^{HuPTM} glycoprotein can be produced in human cell lines by recombinant DNA technology, and the glycoprotein can be administered to patients diagnosed nAMD, diabetic retinopathy, DME, cRVO, pathologic myopia, or polypoidal choroidal vasculopathy by intravitreal administration or to patients diagnosed with cancer, particularly metastatic colon cancer, by infusion or other parenteral administration.

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation, including 2,6-sialylation and sulfation to demonstrate efficacy. In certain embodiments, 0.5% to 1% of the population of VEGF-Trap^{HuPTM} has 2,6-sialylation and/or sulfation. In other embodiments, 2%, from 2% to 5%, or 2% to 10% of the population of the VEGF-Trap^{HuPTM} has 2,6-sialylation and/or sulfation. In certain embodiments, the level of 2,6-sialylation and/or sulfation is significantly higher, such that up to 50%, 60%, 70%, 80%, 90% or even 100% of the molecules contains 2,6-sialylation and/or sulfation. The goal of gene therapy treatment provided herein is to treat retinal neovascularization, and to maintain or improve vision with minimal intervention/invasive procedures or to treat, ameliorate or slow the progression of metastatic colon cancer.

Provided are also methods of treatment with the VEGF-Trap^{HuPTM} in combination with agents or treatments useful for the treatment of eye disease associated with neovascularization or cancer.

Provided also are methods of manufacturing the AAV8 viral vectors containing the VEGF-Trap transgenes and the VEGF-Trap^{HuPTM} protein products.

### 5.1. VEGF-Trap Transgenes

In certain aspects, VEGF-Trap transgenes, as well as constructs encoding the transgene are provided. The VEGF-Trap encoded by the transgene can include, but is not limited to VEGF-Trap^{HuPTM} having the amino acid sequence of aflibercept, as well as VEGF-Trap variants. Aflibercept is a fusion protein which comprises (from amino to carboxy terminus): (i) the Ig-like domain 2 of human Flt-1 (also known as VEGFR1), (ii) the Ig-like domain 3 of human KDR (also known as VEGFR2), and (iii) a human IgG Fc region, particularly the Fc of IgG1. Preferably the VEGF-Trap^{HuPTM} has the amino acid sequence of FIG. 1 (SEQ ID NO: 1, which does not include the leader sequence), which may include the leader sequence of FIG. 1 or an alternative leader sequence as described herein. Variants of the VEGF-Trap can include but are not limited to variants designed to increase stability and residence in the eye, yet reduce the systemic half-life of the transgene product following entry into the systemic circulation. In one embodiment the variant can be a truncated or “Fc-less” VEGF-Trap, may have one or more amino acid substitutions or may have a different IgG Fc domain, such as the Fc of IgG2 or IgG4, or an Ig-like domain from Flt-1, KDR or the like. In another embodiment, the truncated or “Fc-less” VEGF-Trap transgene can be engineered to form a “double dose” construct wherein two “Fc-less” VEGF-Trap transgenes can be inserted into the construct. Alternatively, the variant can be an aflibercept transgene with a modified Fc, wherein the modification disables the FcRn binding site. Such modifications can reduce systemic half-life of the transgene product following entry into the systemic circulation, yet maintain stability and residence in the eye.

VEGF-Trap transgenes refer to transgenes that encode fusion proteins of VEGF receptors 1 and 2, which have been developed for the treatment of several retinal diseases and cancer related to angiogenesis. In one embodiment, VEGF-Trap transgenes can encode recombinant fusion proteins consisting of VEGF-binding regions of the extracellular domains of the human VEGF-receptor fused to the Fc portion of human IgG1. In another embodiment, VEGF-Trap transgenes can encode the signal sequence and domain 2 of VEGF receptor 1 attached to domain 3 of VEGF receptor 2 and a human IgG Fc region (see, for example, Holash et al., 2002, Proc. Natl. Acad. Sci. USA. 99(17):11393). In a further embodiment, the VEGF-Trap transgene can encode a VEGF-Trap with the amino acid sequence of ziv-aflibercept. In another embodiment, the VEGF-Trap transgene can encode Conbercept (de Oliveira Dias et al., 2016, Int J Retin Vitr 2:3).

In a preferred embodiment, the VEGF-Trap transgene can encode the fusion protein of aflibercept. Aflibercept is a fusion protein which comprises (from amino to carboxy terminus): (i) the Ig-like domain 2 of human Flt-1 (aka VEGFR1), (ii) the Ig-like domain 3 of human KDR (aka VEGFR2), and (iii) a human IgG1 Fc region. The amino acid sequence of aflibercept (without any leader sequence) is SEQ ID NO:1 as set forth in Table 1.

Provided are nucleotide sequences encoding the VEGF-Trap transgene products described herein. Preferably, the coding nucleotide sequences are codon optimized for expression in human cells (see, e.g., Quax et al., 2015 Mol. Cell 59:149-161). Algorithms are available for generating sequences that are codon optimized for expression in human cells, for example, the EMBOSS web based translator (http://www.ebi.ac.uk/Tools/st/emboss_backtranseq/), or http://www.geneinfinity.org/sms/sms_backtranslation.html. A codon-optimized nucleotide sequence encoding aflibercept (including the leader sequence) is SEQ ID NO: 2 (with the sequence encoding the leader as in FIG. 1, indicated in italics), with a consensus sequence as SEQ ID NO: 3 (with the sequence encoding the leader sequence from FIG. 1, indicated in italics), as set forth in Table 1. In SEQ ID NO: 3, “r” indicates a purine (g or a); “y” indicates a pyrimidine (t/u or c); “m” is an a or c; “k” is a g or t/u; “s” is a g or c; “w” is an a or t/u; “b” is a g, c or t/u (i.e., not a); “d” is an a, g or t/u (i.e., not c); “h” is an a, c or t/u (i.e., not g); “v” is an a, g or c (i.e., not t nor u); and “n” is a, g, c, t/u, unknown, or other.

TABLE 1

| Description | SEQUENCE |
|---|---|
| Aflibercept | SDTGRPFVEM YSEIPEIIHM TEGRELVIPC RVTSPNITVT LKKFPLDTLI 50 |
| amino acid | PDGKRIIWDS RKGFIISNAT YKEIGLLTCE ATVNGHLYKT NYLTHRQTNT 100 |
| sequence no | IIDVVLSPSH GIELSVGEKL VLNCTARTEL NVGIDFNWEY PSSKHQHKKL 150 |
| leader) | VNRDLKTQSG SEMKKFLSTL TIDGVTRSDQ GLYTCAASSG LMTKKNSTFV 200 |
| SEQ ID NO 1 | RVHEKDKTHT CPPCPAPELL GGPSVFLFPP KPKDTLMISR TPEVTCVVVD 250 |
| | VSHEDPEVKF NWYVDGVEVH NAKTKPREEQ YNSTYRVVSV LTVLHQDWLN 300 |
| | GKEYKCKVSN KALPAPIEKT ISKAKGQPRE PQVYTLPPSR DELTKNQVSL 350 |
| | TCLVKGFYPS DIAVEWESNG QPENNYKTTP PVLDSDGSFF LYSKLTVDKS 400 |
| | RWQQGNVFSC SVMHEALHNH YTQKSLSLSP +/− G or GK |
| Codon optimized | atgtacagaa tgcagctgct gctgctgatc gccctgagcc tggccctggt 50 |
| nucleotide | gaccaacagc agcgacaccg gcagaccctt cgtggagatg tacagcgaga 100 |
| sequence | tccccgagat catccacatg accgagggca gagagctggt gatcccctgc 150 |
| encoding | agagtgacca gccccaacat caccgtgacc ctgaagaagt tccccctgga 200 |
| aflibercept | caccctgatc cccgacggca agagaatcat ctgggacagc agaaagggct 250 |
| (leader in | tcatcatcag caacgccacc tacaaggaga tcggcctgct gacctgcgag 300 |
| italics) | gccaccgtga acggccacct gtacaagacc aactacctga cccacagaca 350 |
| SEQ ID NO: 2 | gaccaacacc atcatcgacg tggtgctgag ccccagccac ggcatcgagc 400 |
| | tgagcgtggg cgagaagctg gtgctgaact gcaccgccag aaccgagctg 450 |
| | aacgtgggca tcgacttcaa ctgggagtac cccagcagca agcaccagca 500 |
| | caagaagctg gtgaacagag acctgaagac ccagagcggc agcgagatga 550 |
| | agaagttcct gagcaccctg accatcgacg gcgtgaccag aagcgaccag 600 |
| | ggcctgtaca cctgcgccgc cagcagcggc ctgatgacca agaagaacag 650 |
| | caccttcgtg agagtgcacg agaaggacaa gacccacacc tgccccccct 700 |
| | gccccgcccc cgagctgctg ggcggcccca gcgtgttcct gttccccccc 750 |
| | aagcccaagg acaccctgat gatcagcaga acccccgagg tgacctgcgt 800 |
| | ggtggtggac gtgagccacg aggaccccga ggtgaagttc aactggtacg 850 |
| | tggacggcgt ggaggtgcac aacgccaaga ccaagcccag agaggagcag 900 |
| | tacaacagca cctacagagt ggtgagcgtg ctgaccgtgc tgcaccagga 950 |
| | ctggctgaac ggcaaggagt acaagtgcaa ggtgagcaac aaggccctgc 1000 |
| | ccgcccccat cgagaagacc atcagcaagg ccaagggcca gcccagagag 1050 |
| | ccccaggtgt acaccctgcc ccccagcaga gacgagctga ccaagaacca 1100 |
| | ggtgagcctg acctgcctgg tgaagggctt ctaccccagc gacatcgccg 1150 |
| | tggagtggga gagcaacggc cagcccgaga acaactacaa gaccaccccc 1200 |
| | cccgtgctgg acagcgacgg cagcttcttc ctgtacagca agctgaccgt 1250 |
| | ggacaagagc agatggcagc agggcaacgt gttcagctgc agcgtgatgc 1300 |
| | acgaggccct gcacaaccac tacacccaga agagcctgag cctgagcccc 1350 |
| | +/− ggc or ggc aag |
| Codon optimized | atgtaymgna tgcarytnyt nytnytnath gcnytnwsny tngcnytngt 50 |
| consensus | nacnaaywsn wsngayacng gnmgnccntt ygtngaratg taywsngara 100 |
| sequence | thccngarat hathcayatg acngarggnm gngarytngt nathccntgy 150 |
| encoding | mgngtnacnw snccnaayat hacngtnacn ytnaaraart tyccnytnga 200 |
| aflibercept | yacnytnath ccngayggna armgnathat htgggaywsn mgnaarggnt 250 |
| (leader in | tyathathws naaygcnacn tayaargara thggnytnyt nacntgygar 300 |
| italics) | gcnacngtna ayggncayyt ntayaaracn aaytayytna cncaymgnca 350 |
| SEQ ID NO: 3 | racnaayacn athathgayg tngtnytnws nccnwsncay ggnathgary 400 |
| | tnwsngtngg ngaraarytn gtnytnaayt gyacngcnmg nacngarytn 450 |
| | aaygtnggna thgayttyaa ytgggartay ccnwsnwsna arcaycarca 500 |
| | yaaraarytn gtnaaymgng ayytnaarac ncarwsnggn wsngaratga 550 |
| | araarttyyt nwsnacnytn acnathgayg gngtnacnmg nwsngaycar 600 |
| | ggnytntaya cntgygcngc nwsnwsnggn ytnatgacna araaraayws 650 |
| | nacnttygtn mgngtncayg araargayaa racncayacn tgyccnccnt 700 |
| | gyccngcncc ngarytnytn ggnggnccnw sngtnttyyt nttyccnccn 750 |
| | aarccnaarg ayacnytnat gathwsnmgn acnccngarg tnacntgygt 800 |
| | ngtngtngay gtnwsncayg argayccnga rgtnaartty aaytggtayg 850 |
| | tngayggngt ngargtncay aaygcnaara cnaarccnmg ngargarcar 900 |
| | tayaaywsna cntaymgngt ngtnwsngtn ytnacngtny tncaycarga 950 |
| | ytggytnaay ggnaargart ayaartgyaa rgtnwsnaay aargcnytnc 1000 |
| | cngcnccnat hgaraaracn athwsnaarg cnaarggnca rccnmgngar 1050 |
| | ccncargtnt ayacnytncc nccnwsnmgn gaygarytna cnaaraayca 1100 |
| | rgtnwsnytn acntgyytng tnaarggntt ytayccnwsn gayathgcng 1150 |
| | tngartggga rwsnaayggn carccngara ayaaytayaa racnacnccn 1200 |
| | ccngtnytng aywsngaygg nwsnttytty ytntaywsna arytnacngt 1250 |
| | ngayaarwsn mgntggcarc arggnaaygt nttywsntgy wsngtnatgc 1300 |
| | aygargcnyt ncayaaycay tayacncara arwsnytnws nytnwsnccn 1350 |
| | +/− ggn or ggn aan |

As shown in FIG. 1, the human Flt-1 sequence in the aflibercept sequence is amino acids 1 to 102, the KDR sequence is amino acids 103 to 205, and the IgG1 Fc domain is amino acids 206 to 431, with the IgG1 Fc hinge region being amino acids 206 to 222, of SEQ ID NO:1. FIG. 1 provides the amino acid sequence of the fusion protein of aflibercept with the Flt-1 leader sequence, MVSYWDTGVLLCALLSCLLLTGSSSG (SEQ ID NO: 36), at the N-terminus. In another embodiment, the VEGF-Trap transgene can encode the fusion protein of aflibercept with the human KDR signal sequence, MQSKVLLAVALWLCVETRA (SEQ ID NO: 37), or alternatively, MRMQLLLLIALSLALVTNS (SEQ ID NO: 39), a heterologous leader sequence, or MYRMQLLLLIALSLALVTNS (SEQ ID NO: 38), an alternate heterologous leader sequence (see FIG. 2). Leader sequences are also disclosed infra that are useful for the expression and appropriate post-translational processing and modification of the VEGF-Trap^{HuPTM} in eitherhuman retinal cells or human liver cells, see Tables 3 and 4, respectively.

In certain embodiments, the VEGF-Trap^{HuPTM} transgene encodes a VEGF-Trap comprising an amino acid sequence that is at least 85%, 86%, 87%, 88%, 89%, 90%, 91%, 92%, 93%, 94%, 95%, 96%, 97%, 98% or 99% identical to the amino acid sequence of SEQ ID NO:1 and having the biological activity of a VEGF-trap fusion protein such as aflibercept.

Variants of the VEGF-Trap can include but are not limited to variants designed to increase stability and residence in the eye, yet reduce the systemic half-life of the transgene product following entry into the systemic circulation. In one embodiment the variant can be a truncated or “Fc-less” VEGF-Trap (that may or may not contain the hinge region of the Fc domain). In another embodiment, the truncated or “Fc-less” or Fc⁽⁻⁾ VEGF-Trap transgene can be engineered to form a “double dose” construct wherein two “Fc-less” VEGF-Trap transgenes can be inserted into and expressed from the construct as described infra. Alternatively, the variant can be the fusion protein of aflibercept transgene with a modified Fc, such as a truncated Fc with a C-terminal lysine (-K) or glycine-lysine (-GK) deletion, or a modification that disables the FcRn binding site. Such modifications can reduce systemic half-life of the transgene product following entry into the systemic circulation,yet maintain stability and residence in the eye. VEGF-Trap transgenes with a modified Fc should make the protein safer, since prolonged residence of anti-VEGF agents in the systemic circulation is associated with hemorrhagic and thromboembolic complications. In one embodiment, patients administered aflibercept transgenes with a modified Fc experience less hemorrhagic and/or thromboembolic complications. (See, for example, Ding et al., 2017, MAbs 9:269-284; Kim, 1999, Eur J Immunol 29:2819; Andersen, 2012, J Biol Chem 287: 22927-22937; and Regula, 2016, EMBO Mol Med 8: 1265-1288.)

In one embodiment, the VEGF-Trap variant can be the fusion protein of aflibercept with a modified IgG Fc. For example, the C-terminal lysines (-K) conserved in the heavy chain genes of all human IgG subclases generally absent from IgG in serum—the C-terminal lysines are cleaved off in circulation, resulting in a heterogenous population of circulating IgGs. (van den Bremer et al., 2015, mAbs 7:672-680). The DNA encoding the C-terminal lysine (-K) or glycine-lysine (-GK) of the Fc of VEGF-Trap can be deleted to produce a more homogeneous transgene product in situ. (see, Hu et al., 2017 Biotechnol. Prog. 33: 786-794 which is incorporated by reference herin in its entirety). In another embodiment the Fc modification can be a mutation that disables the FcRn binding site, thereby, reducing the systemic half-life of the protein. These mutations include mutations at I253, H310, and/or H435 and, more specifically, include I253A, H310A, and/or H435Q or H435A, using the usual numbering of the positions in the IgG1 heavy chain. These positions correspond to I238, H295 and H420 in the VEGF-Trap^{HuPTM} of FIG. 1. Thus, provided are VEGF-Trap^{HuPTM} comprising an IgG1 Fc domain with a substitution alanine for isoleucine at position 238, the substitution of alanine for histidine at position 295 and/or a substitution of glutamine or alanine for histidine at position 420 of SEQ ID NO:1 (or the position corresponding thereto in a different VEGF trap protein as determined by routine sequence alignment). In certain embodiments, the VEGF-Trap^{HuPTM} has one, two or three of the mutations I238A, H295A and H435Q or H420A. An exemplary VEGF-Trap^{HuPTM} amino acid sequence of a fusion protein having the amino acid sequence of aflibercept with an alanine or glutamine substitution at position 420 is provided in FIG. 3.

In certain embodiments, the VEGF-Trap^{HuPTM} is a variant of the amino acid sequence of aflibercept that either does not comprise the IgG1 Fc domain (amino acids 206 to 431 of SEQ ID NO: 1), resulting in a fusion protein of amino acids 1 to 205 of SEQ ID NO:1. In specific embodiments, the VEGF-Trap^{HuPTM} does not comprise the IgG1 Fc domain and also may or may not have the terminal lysine of the KDR sequence (i.e., amino acid 205 of SEQ ID NO:1) resulting in a fusion protein of amino acids 1 to 204 of SEQ ID NO:1. Alternatively, the VEGF-Trap^{HuPTM} has all or a portion of the hinge region of IgG1 Fc at the C-terminus of the protein, as indicated in FIG. 4. In specific embodiments, the C-terminal sequence may be DKTHT (SEQ ID NO: 44) or DKTHL (SEQ ID NO: 45) (amino acids 206 to 210 of SEQ ID NO:1, optionally with a leucine substituted for the threonine at position 210), resulting in a VEGF-trap with an amino acid sequence of positions 1 to 210 of SEQ ID NO: 1; or may be DKTHTCPPCPA (SEQ ID NO: 46) (amino acids 206 to 216 of SEQ ID NO:1), resulting in a VEGF-Trap with an amino acid sequence of positions 1 to 216 of SEQ ID NO: 1; or DKTHTCPPCPAPELLGG (SEQ ID NO: 47) (amino acids 206 to 222 of SEQ ID NO:1), resulting in a VEGF-Trap with an amino acid sequence of positions 1 to 222 of SEQ ID NO:1); or DKTHTCPPCPAPELLGGPSVFL (SEQ ID NO: 48) (amino acids 206 to 227), resulting in a VEGF-Trap with an amino acid sequence of positions 1 to 227 of SEQ ID NO:1 (and may also include a leader sequence at the N-terminus). The cysteine residues in the hinge region may promote the formation of inter-chain disulfide bonds whereas fusion proteins that do not contain all or a cysteine-containing portion of the hinge region may not form inter chain bonds but only intra-chain bonds. This Fc-less or Fc⁽⁻⁾ VEGF-Trap transgene may be used in tandem in an expression construct comprising and expressing two copies of the VEGF-Trap transgene. The Fc-less transgene accommodating the size restrictions by adding a second copy of the transgene in, for example, an AAV8 viral vector.

In alternative embodiments, the VEGF-Trap^{HuPTM} has an Fc domain or other domain sequence substituted for the IgG1 Fc domain that may improve or maintain the stability of the VEGF-Trap^{HuPTM} in the eye while reducing the half-life of the VEGF-Trap^{HuPTM} once it has entered the systemic circulation, reducing the potential for adverse effects. In particular embodiments, the VEGF-Trap^{HuPTM} has substituted for amino acids 206 to 431 of SEQ ID NO:1 an alternative Fc domain, including an IgG2 Fc or IgG4 Fc domain as set forth in FIGS. 7A and B, respectively, where the hinge sequence is indicated in italics. Sequences are presented in Table 2 below. Variants include Fc domains with all or a portion of the hinge regions, or none of the hinge region. In certain embodiments where interchain disulfide bonds are not desired, one or more of the cysteine residues within the hinge region may be substituted with a serine, for example at positions 210 and 213 of the IgG4 Fc hinge (see FIGS. 7F and H, with substitutions underlined). The amino acid sequences of exemplary transgene products with IgG2 or IgG4 Fc domains are presented in FIGS. 7C-H.

In other alternative embodiments, the VEGF-Trap^{HuPTM} has substituted for the IgG1 Fc domain, one or more of the Ig-like domains of human Flt-1 or human KDR, or a combination thereof. The amino acid sequences of the extracellular domains (and signal sequences) of human Flt 1 and human KDR are presented in FIGS. 8A and 8B, respectively, with the Ig-like domains indicated in color text. Provided are transgene products in which the C-terminal domain consists of or comprises one, two, three or four of the Ig-like domains of human Flt1, particularly, at least Ig-like domains 2 and 3; or one, two, three or four of the Ig-like domains of human KDR, particularly, at least domains 3, 4, and/or 5. In a specific embodiment, the transgene product has a C-terminal domain with the KDR Ig-like domains 3, 4 and 5 and the Flt1 Ig-like domain 2.

Exemplary sequences that can be used to substitute for the IgG1 Fc domain of SEQ ID NO:1 are provided in Table 2 below. The amino acid sequences of exemplary transgene products that have Flt-1 and/or KDR Ig-like domains substituted for the IgG1 Fc domain of SEQ ID NO:1 are provided in FIGS. 8C and D.

TABLE 2
IgG1 Fc replacement sequences

| Alternative | SEQ | |
|---|---|---|
| to IgG1 Fc | ID | |
| domain | NO: | Amino Acid Sequence |
| IgG2 Fc | 19 | ASTKGPSVFP LAPCSRSTSE STAALGCLVK DYFPEPVTVS WNSGALTSGV 50 |
| sequence | | HTFPAVLQSS GLYSLSSVVT VPSSNFGTQT YTCNVDHKPS NTKVDKTVER 100 |
| | | KCCVECPPCP APPVAGPSVF LFPPKPKDTL MISRTPEVTC VVVDVSHEDP 150 |
| | | EVQFNWYVDG VEVHNAKTKP REEQFNSTFR VVSVLTVVHQ DWLNGKEYKC 200 |
| | | KVSNKGLPAP IEKTISKTKG QPREPQVYTL PPSREEMTKN QVSLTCLVKG 250 |
| | | FYPSDISVEW ESNGQPENNY KTTPPMLDSD GSFFLYSKLT VDKSRWQQGN 300 |
| | | VFSCSVMHEA LHNHYTQKSL SLSP +/− G or GK |
| IgG2 Fc | 49 | VECPPCPAPP VAGPSVFLFP PKPKDTLMIS RTPEVTCVVV DVSHEDPEVQ 50 |
| Sequence | | FNWYVDGVEV HNAKTKPREE QFNSTFRVVS VLTVVHQDWL NGKEYKCKVS 100 |
| partial hinge | | NKGLPAPIEK TISKTKGQPR EPQVYTLPPS REEMTKNQVS LTCLVKGFYP 150 |
| (2 di-S | | SDISVEWESN GQPENNYKTT PPMLDSDGSF FLYSKLTVDK SRWQQGNVFS 200 |
| bonds) | | CSVMHEALHN HYTQKSLSLS P +/− G or GK |
| IgG2 Fc | 50 | ERKCCVECPP CPAPPVAGPS VFLFPPKPKD TLMISRTPEV TCVVVDVSHE 50 |
| Sequence | | DPEVQFNWYV DGVEVHNAKT KPREEQFNST FRVVSVLTVV HQDWLNGKEY 100 |
| entire hinge | | KCKVSNKGLP APIEKTISKT KGQPREPQVY TLPPSREEMT KNQVSLTCLV 150 |
| (4-di S | | KGFYPSDISV EWESNGQPEN NYKTTPPMLD SDGSFFLYSK LTVDKSRWQQ 200 |
| bonds) | | GNVFSCSVMH EALHNHYTQK SLSLSP +/− G or GK |
| IgG4 Fc | 20 | ASTKGPSVFP LAPCSRSTSE STAALGCLVK DYFPEPVTVS WNSGALTSGV 50 |
| Sequence | | HTFPAVLQSS GLYSLSSVVT VPSSSLGTKT YTCNVDHKPS NTKVDKRVES 100 |
| | | KYGPPCPSCP APEFLGGPSV FLFPPKPKDT LMISRTPEVT CVVVDVSQED 150 |
| | | PEVQFNWYVD GVEVHNAKTK PREEQFNSTY RVVSVLTVLH QDWLNGKEYK 200 |
| | | CKVSNKGLPS SIEKTISKAK GQPREPQVYT LPPSQEEMTK NQVSLTCLVK 250 |
| | | GFYPSDIAVE WESNGQPENN YKTTPPVLDS DGSFFLYSRL TVDKSRWQEG 300 |
| | | NVFSCSVMHE ALHNHYTQKS LSLSL +/− G or GK |
| IgG4 Fc | 51 | YGPPCPSCPA PEFLGGPSVF LFPPKPKDTL MISRTPEVTC VVVDVSQEDP 50 |
| region | | EVQFNWYVDG VEVHNAKTKP REEQFNSTYR VVSVLTVLHQ DWLNGKEYKC 100 |
| partial hinge | | KVSNKGLPSS IEKTISKAKG QPREPQVYTL PPSQEEMTKN QVSLTCLVKG 150 |
| | | FYPSDIAVEW ESNGQPENNY KTTPPVLDSD GSFFLYSRLT VDKSRWQEGN 200 |
| | | VFSCSVMHEA LHNHYTQKSL SLSL +/− G or GK |
| IgG4 Fc | 52 | YGPPSPSSPA PEFLGGPSVF LFPPKPKDTL MISRTPEVTC VVVDVSQEDP 50 |
| partial hinge | | EVQFNWYVDG VEVHNAKTKP REEQFNSTYR VVSVLTVLHQ DWLNGKEYKC 100 |
| regions with | | KVSNKGLPSS IEKTISKAKG QPREPQVYTL PPSQEEMTKN QVSLTCLVKG 150 |
| substitutions | | FYPSDIAVEW ESNGQPENNY KTTPPVLDSD GSFFLYSRLT VDKSRWQEGN 200 |
| | | VFSCSVMHEA LHNHYTQKSL SLSL +/− G or GK |
| IgG4 Fc with | 53 | ESKYGPPCPS CPAPEFLGGP SVFLFPPKPK DTLMISRTPE VTCVVVDVSQ 50 |
| full hinge | | EDPEVQFNWY VDGVEVHNAK TKPREEQFNS TYRVVSVLTV LHQDWLNGKE 100 |
| region | | YKCKVSNKGL PSSIEKTISK AKGQPREPQV YTLPPSQEEM TKNQVSLTCL 150 |
| | | VKGFYPSDIA VEWESNGQPE NNYKTTPPVL DSDGSFFLYS RLTVDKSRWQ 200 |
| | | EGNVFSCSVM HEALHNHYTQ KSLSLSL +/− G or GK |
| IgG4 Fc with | 54 | ESKYGPPSPS CPAPEFLGGP SVFLFPPKPK DTLMISRTPE VTCVVVDVSQ 50 |
| full hinge | | EDPEVQFNWY VDGVEVHNAK TKPREEQFNS TYRVVSVLTV LHQDWLNGKE 100 |
| region and | | YKCKVSNKGL PSSIEKTISK AKGQPREPQV YTLPPSQEEM TKNQVSLTCL 150 |
| substitution | | VKGFYPSDIA VEWESNGQPE NNYKTTPPVL DSDGSFFLYS RLTVDKSRWQ 200 |
| | | EGNVFSCSVM HEALHNHYTQ KSLSLSL +/− G or GK |
| Flt-1 | 55 | PFVEMYSEIP EIIHMTEGRE LVIPCRVTSP NITVTLKKFP LDTLIPDGKR 50 |
| domains | | IIWDSRKGFI ISNATYKEIG LLTCEATVNG HLYKTNYLTH RQTNTIIDVQ 100 |
| (amino acids | | ISTPRPVKLL RGHTLVLNCT ATTPLNTRVQ MTWSYPDEKN KRASVRRRID 150 |
| 134 to 347 of | | QSNSHANIFY SVLTIDKMQN KDKGLYTCRV RSGPSFKSVN TSVHIYDKAF 200 |
| Flt-1 of FIG. | | ITVK |
| 8A) | | |
| KDR | 56 | PFVAFGSGME SLVEATVGER VRIPAKYLGY PPPEIKWYKN GIPLESNHT 50 |
| domains | | IKAGHVLTIM EVSERDTGNY TVILTNPISK EKQSHVVSLV VYVPPQIGE 100 |
| (amino acids | | KSLISPVDSY QYGTTQTLTC TVYAIPPPHH IHWYWQLEEE CANEPSQAV 150 |
| 328 to 548 of | | SVTNPYPCEE WRSVEDFQGG NKIEVNKNQF ALIEGKNKTV STLVIQAAN 200 |
| FIG. 8A) | | VSALYKCEAV NKVGRGERVI SFHVT |

### 5.2 VEGF-Trap^{HuPTM} Constructs

In certain aspects, provided herein are constructs for the expression of VEGF-Trap transgenes in human retinal cells or in human liver cells. The constructs can include the transgene and appropriate expression control elements for expression in retinal cells or in liver cells. In one aspect, the vector is a viral vector comprising the VEGF-Trap transgene and expression control element. In a specific aspect, the viral vector is an AAV vector which comprises the VEGF-Trap transgene, which includes a nucleotide sequence encoding a signal sequence. In a more specific embodiment, an AAV vector comprising a nucleotide sequence encoding a VEGF-Trap transgene and a signal sequence is provided. In another specific embodiment, an AAV8 vector comprising a transgene encoding a VEGF-Trap protein and a signal sequence are provided. In one embodiment, an AAV8 vector comprising a transgene encoding a VEGF-Trap^{HuPTM} having an amino acid sequence of SEQ ID NO:1 and a signal sequence is provided. In specific embodiments, the AAV8 vector further comprises a regulatory sequence, such as a promoter, operably linked to the transgene that allows for expression in retinal cells or liver cells. The promoter may be a constitutive promoter, for example, the CB7 promoter. Alternatively, and particularly for use in treating cancer where it may be desireable to turn off transgene expression once the cancer has been treated or if side effects arise, an inducible promoter may be used, for example, a hypoxia-inducible or rapamycin inducible promoter as described herein.

The recombinant vector used for delivering the transgene should have a tropism for retinal cells or for liver cells. These can include non-replicating recombinant adeno-associated virus vectors (“rAAV”), particularly those bearing an AAV8 capsid, or variants of an AAV8 capsid are preferred. However, other viral vectors may be used, including but not limited to lentiviral vectors, vaccinia viral vectors, or non-viral expression vectors referred to as “naked DNA” constructs. Preferably, the VEGF-Trap^{HuPTM} transgene should be controlled by appropriate expression control elements, for example, the ubiquitous CB7 promoter (a chicken β-actin promoter and CMV enhancer), or tissue-specific promoters such as RPE-specific promoters e.g., the RPE65 promoter, or cone-specific promoters, e.g., the opsin promoter, or liver-specific promoters, such as the TBG (Thyroxine-binding Globulin) promoter, the APOA2 promoter, SERPINA1 (hAAT) promoter, or mIR122 promoter, or inducible promoters, such as a hypoxia-inducible promoter or a rapamycin-inducible promoter, to name a few. The construct can include other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor /immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal. See, e.g., Powell and Rivera-Soto, 2015, Discov. Med., 19(102):49-57.

For use in the methods provided herein are viral vectors or other DNA expression constructs encoding a VEGF-Trap. The viral vectors and other DNA expression constructs provided herein include any suitable method for delivery of a transgene to a target cell, such as human retinal cells, including human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); retinal pigment epithelial cells; and human liver cells. The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted to, for example, human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); retinal pigment epithelial cells; and human liver cells.

In some aspects, the disclosure provides for a nucleic acid for use, wherein the nucleic acid encodes a VEGF-Trap or VEGF-Trap^{HuPTM} operatively linked to a promoter selected from the group consisting of: CB7 promoter, cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter, opsin promoter, the TBG (Thyroxine-binding Globulin) promoter, the APOA2 promoter, SERPINA1 (hAAT) promoter, MIR122 promoter, hypoxia-inducible promoter, or rapamycin inducible promoter.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., a VEGF-Trap transgene), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

In a specific embodiment, the constructs described herein comprise the following components: (1) AAV2 inverted terminal repeats that flank the expression cassette; (2) Control elements, which include a) the CB7 promoter, comprising the CMV enhancer/chicken β-actin promoter, b) a chicken β-actin intron and c) a rabbit β-globin poly A signal; and (3) nucleic acid sequences coding for a VEGF-Trap. In a specific embodiment, the constructs described herein comprise the following components: (1) AAV2 inverted terminal repeats that flank the expression cassette; (2) Control elements, which include a) a hypoxia-inducible promoter, b) a chicken β-actin intron and c) a rabbit β-globin poly A signal; and (3) nucleic acid sequences coding for a VEGF-Trap.

5.2.1 mRNA Vectors

In certain embodiments, as an alternative to DNA vectors, the vectors provided herein are modified mRNA encoding for the gene of interest (e.g., the transgene, for example, VEGF-Trap). The synthesis of modified and unmodified mRNA for delivery of a transgene to retinal or liver cells is taught, for example, in Hansson et al., J. Biol. Chem., 2015, 290(9):5661-5672, which is incorporated by reference herein in its entirety. In certain embodiments, provided herein is a modified mRNA encoding for a VEGF-Trap.

5.2.2 Viral Vectors

Viral vectors include adenovirus, adeno-associated virus (AAV, e.g., AAV8), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV)-based and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In more specific embodiments, the envelope protein is VSV-G protein.

In certain embodiments, the viral vectors provided herein are HIV based viral vectors. In certain embodiments, HIV-based vectors provided herein comprise at least two polynucleotides, wherein the gag and pol genes are from an HIV genome and the env gene is from another virus.

In certain embodiments, the viral vectors provided herein are herpes simplex virus-based viral vectors. In certain embodiments, herpes simplex virus-based vectors provided herein are modified such that they do not comprise one or more immediately early (IE) genes, rendering them non-cytotoxic.

In certain embodiments, the viral vectors provided herein are MLV based viral vectors. In certain embodiments, MLV-based vectors provided herein comprise up to 8 kb of heterologous DNA in place of the viral genes.

In certain embodiments, the viral vectors provided herein are lentivirus-based viral vectors. In certain embodiments, lentiviral vectors provided herein are derived from human lentiviruses. In certain embodiments, lentiviral vectors provided herein are derived from non-human lentiviruses. In certain embodiments, lentiviral vectors provided herein are packaged into a lentiviral capsid. In certain embodiments, lentiviral vectors provided herein comprise one or more of the following elements: long terminal repeats, a primer binding site, a polypurine tract, att sites, and an encapsidation site.

In certain embodiments, the viral vectors provided herein are alphavirus-based viral vectors. In certain embodiments, alphavirus vectors provided herein are recombinant, replication-defective alphaviruses. In certain embodiments, alphavirus replicons in the alphavirus vectors provided herein are targeted to specific cell types by displaying a functional heterologous ligand on their virion surface.

The recombinant vector used for delivering the transgene includes non-replicating recombinant adeno-associated virus vectors (“rAAV”). rAAVs are particularly attractive vectors for a number of reasons—they can transduce non-replicating cells, and therefore, can be used to deliver the transgene to tissues where cell division occurs at low levels; they can be modified to preferentially target a specific organ of choice; and there are hundreds of capsid serotypes to choose from to obtain the desired tissue specificity, and/or to avoid neutralization by pre-existing patient antibodies to some AAVs.

In certain embodiments, the viral vectors provided herein are AAV based viral vectors. In preferred embodiments, the viral vectors provided herein are AAV8 based viral vectors. In certain embodiments, the AAV8 based viral vectors provided herein retain tropism for retinal cells. In certain embodiments, the AAV8 based viral vectors provided herein retain tropism for liver cells. In certain embodiments, the AAV-based vectors provided herein encode the AAV rep gene (required for replication) and/or the AAV cap gene (required for synthesis of the capsid proteins). In preferred embodiments, the AAV vectors are non-replicating and do not include the nucleotide sequences encoding the rep or cap proteins (these are supplied by the packaging cells in the manufacture of the rAAV vectors). Multiple AAV serotypes have been identified. In certain embodiments, AAV-based vectors provided herein comprise components from one or more serotypes of AAV. In certain embodiments, AAV based vectors provided herein comprise capsid components from one or more of AAV1, AAV2, AAV3, AAV4, AAV5, AAV6, AAV7, AAV8, AAV9, AAV10, AAV11, AAVrh20 or AAVrh10. In preferred embodiments, AAV based vectors provided herein comprise components from one or more of AAV8, AAV9, AAV10, AAV11, AAVrh20 or AAVrh10 serotypes.

In certain embodiments, the AAV that is used in the compositions and methods described herein is Anc80 or Anc80L65, as described in Zinn et al., 2015, Cell Rep. 12(6): 1056-1068, which is incorporated by reference in its entirety. In certain embodiments, the AAV that is used in the compositions and methods described herein comprises one of the following amino acid insertions: LGETTRP (SEQ ID NO: 57) or LALGETTRP (SEQ ID NO: 58), as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is AAV.7m8 (including variants thereof), as described in U.S. Pat. Nos. 9,193,956; 9,458,517; and 9,587,282; US patent application publication no. 2016/0376323, and International Publication WO 2018/075798, each of which is incorporated herein by reference in its entirety. In certain embodiments, the AAV that is used in the compositions and methods described herein is any AAV disclosed in U.S. Pat. No. 9,585,971, such as AAV-PHP.B. In certain embodiments, the AAV used in the compositions and methods described herein is an AAV2/Rec2 or AAV2/Rec3 vector, which have hybrid capsid sequences derived from AAV8 capsids and capsids of serotypes cy5, rh20 or rh39 as described in Charbel Issa et al., 2013, PLoS One 8(4): e60361, which is incorporated by reference herein for these vectors. In certain embodiments, the AAV that is used in the methods described herein is an AAV disclosed in any of the following patents and patent applications, each of which is incorporated herein by reference in its entirety: U.S. Pat. Nos. 7,906,111; 8,524,446; 8,999,678; 8,628,966; 8,927,514; 8,734,809; 9,284,357; 9,409,953; 9,169,299; 9,193,956; 9,458,517; and 9,587,282 US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

AAV8-based viral vectors are used in certain of the compositions and methods described herein. Nucleic acid sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV8)-based viral vectors encoding a transgene (e.g., a VEGF-Trap). In specific embodiments, provided herein are AAV8-based viral vectors encoding VEGF-Trap. In more specific embodiments, provided herein are AAV8-based viral vectors encoding the fusion protein of aflibercept.

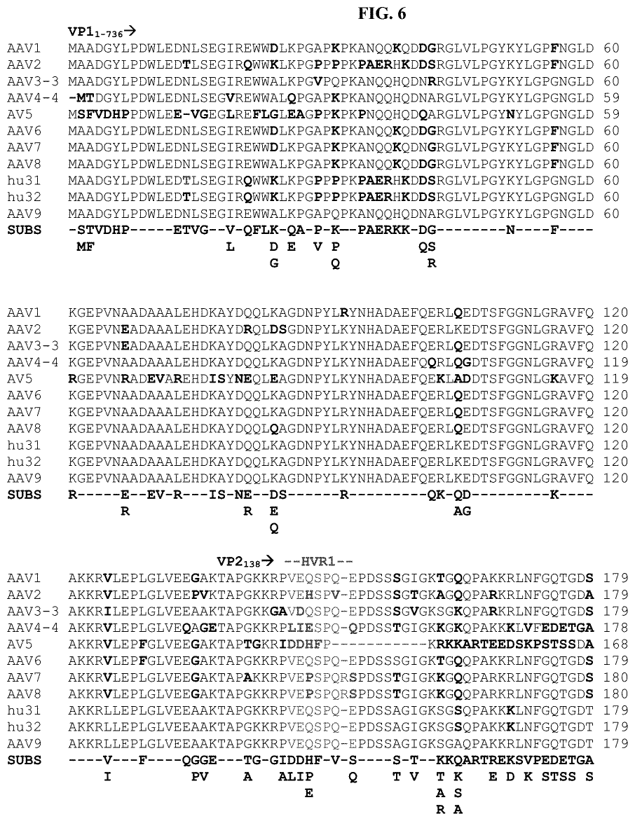

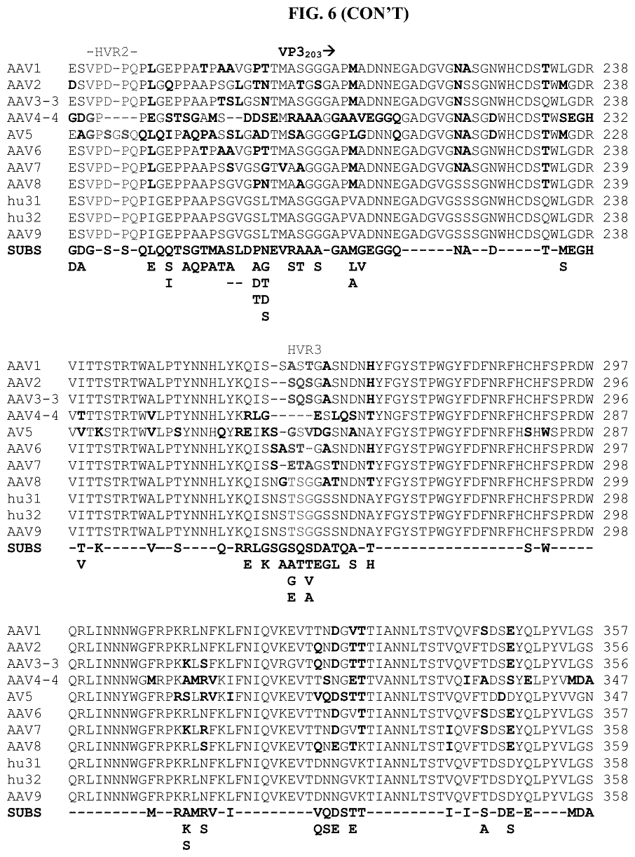

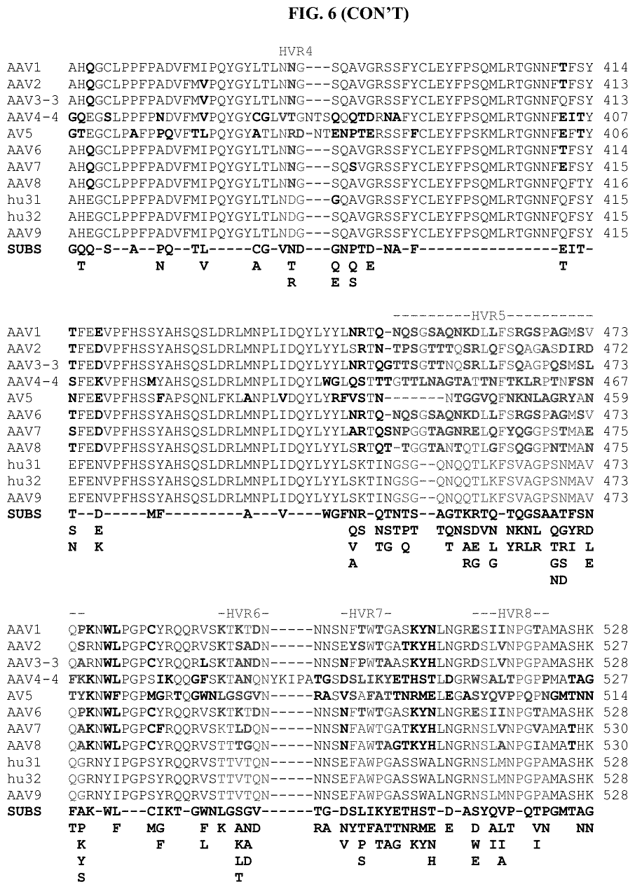

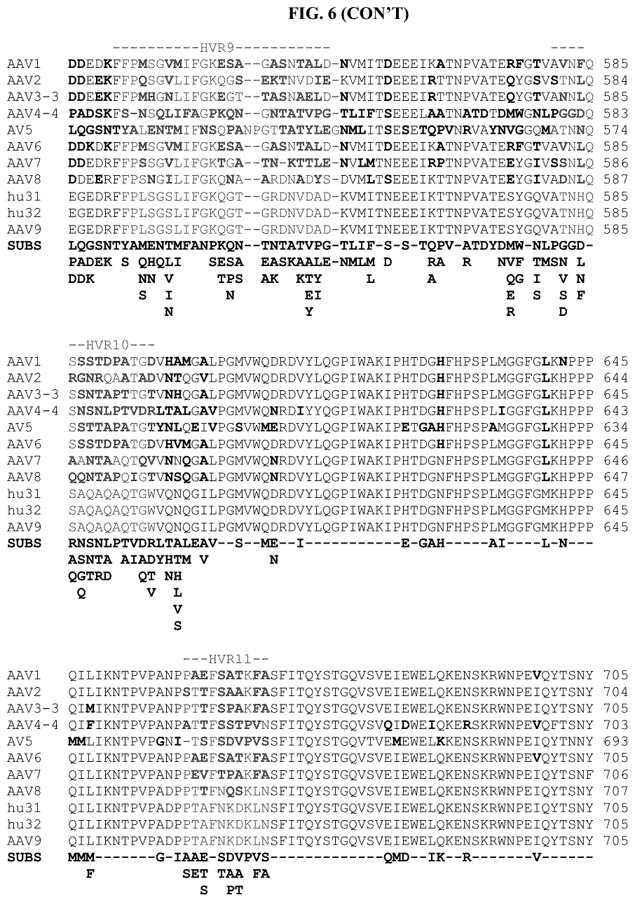

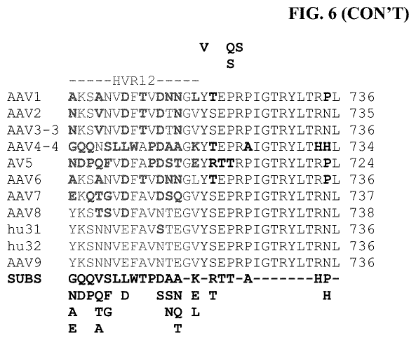

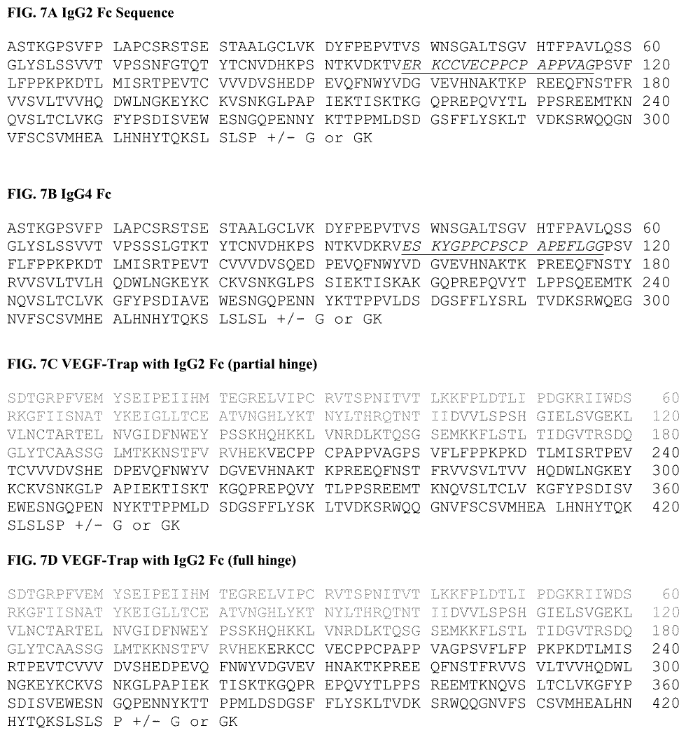

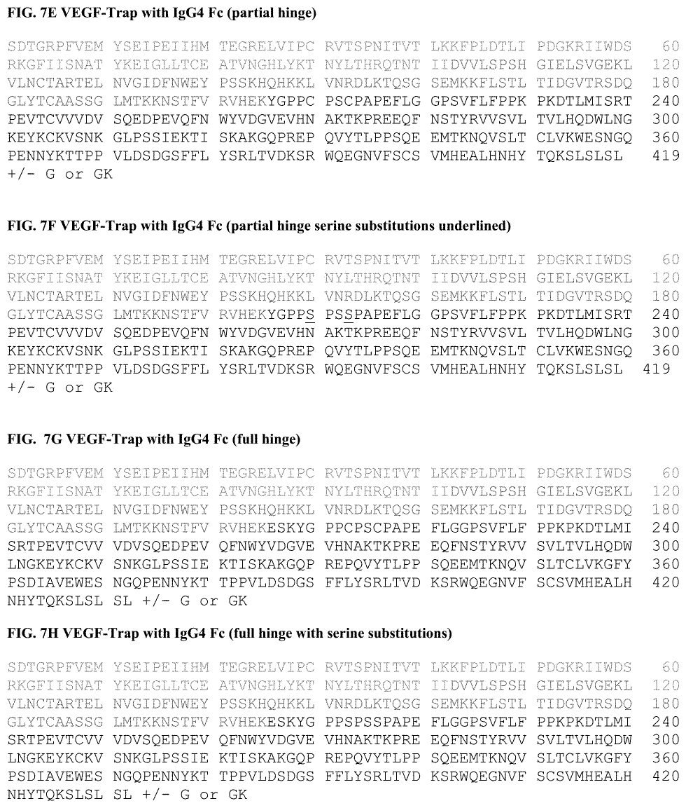

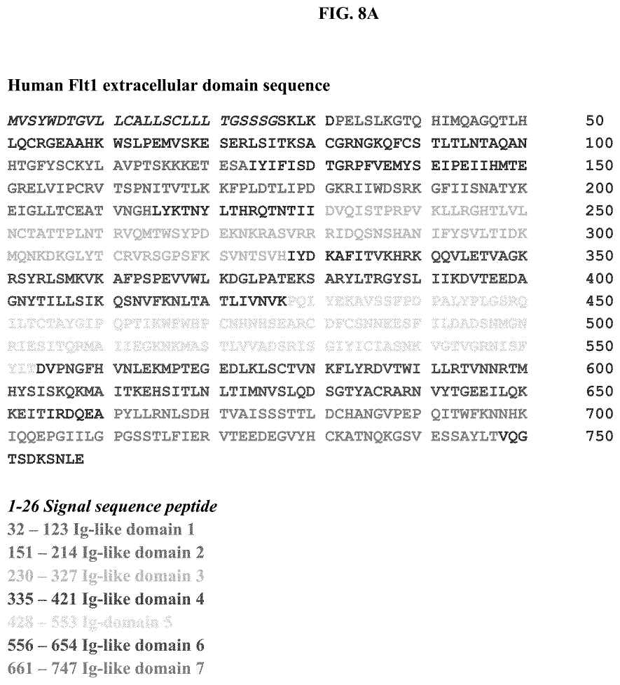

Provided in particular embodiments are AAV8 vectors comprising a viral genome comprising an expression cassette for expression of the transgene, under the control of regulatory elements and flanked by ITRs and a viral capsid that has the amino acid sequence of the AAV8 capsid protein or is at least 95%, 96%, 97%, 98%, 99% or 99.9% identical to the amino acid sequence of the AAV8 capsid protein (SEQ ID NO: 11) while retaining the biological function of the AAV8 capsid. In certain embodiments, the encoded AAV8 capsid has the sequence of SEQ ID NO: 11 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8 capsid. FIG. 6 provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV8 vector comprises an AAV8 capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions identified in the SUBS row of FIG. 6 that are not present at that position in the native AAV8 sequence.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82; McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

Nucleic acid sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety.

The invention will be illustrated by exemplary embodiments but is not meant to be so limited, while the embodiments relate to rAAV vectors, different transgene delivery systems such as adenovirus, lentivirus, vaccinia virus and/or non-viral expression vectors such as “naked” DNA constructs could be used. Expression of the transgene can be controlled by constitutive or tissue-specific expression control elements.

In certain embodiments, the viral vectors used in the methods described herein are adenovirus based viral vectors. A recombinant adenovirus vector may be used to transfer in the VEGF-Trap. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In certain embodiments, the viral vectors used in the methods described herein are lentivirus based viral vectors. A recombinant lentivirus vector may be used to transfer in the VEGF-Trap. Four plasmids are used to make the construct: Gag/pol sequence containing plasmid, Rev sequence containing plasmids, Envelope protein containing plasmid (i.e. VSV-G), and Cis plasmid with the packaging elements and the VEGF-Trap gene.

For lentiviral vector production, the four plasmids are co-transfected into cells (i.e., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10(2):32-43, both of which are incorporated by reference herein in their entireties.

In a specific embodiment, a vector for use in the methods described herein is one that encodes a VEGF-Trap such that, upon introduction of the vector into a relevant cell (e.g., a retinal cell in vivo or in vitro), a glycosylated and or tyrosine sulfated variant of the VEGF-Trap is expressed by the cell. In a specific embodiment, the expressed VEGF-Trap^{HuPTM} comprises a glycosylation and/or tyrosine sulfation pattern as described herein.

5.2.3 Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters. In certain embodiments, the promoter is a constitutive promoter. In certain embodiments, the promoter is a CB7 promoter (see Dinculescu et al., 2005, Hum Gene Ther 16: 649-663, incorporated by reference herein in its entirety). In some embodiments, the CB7 promoter includes other expression control elements that enhance expression of the transgene driven by the vector. In certain embodiments, the other expression control elements include chicken β-actin intron and/or rabbit β-globin polA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs. In certain embodiments, the vectors provided herein comprise one or more tissue specific promoters (e.g., a retinal pigment epithelial cell-specific promoter or liver-specific promoter). In certain embodiments, the viral vectors provided herein comprise a RPE65 promoter. In certain embodiments, the viral vectors provided herein comprise a TBG (Thyroxine-binding Globulin) promoter, a APOA2 promoter, a SERPINA1 (hAAT) promoter, or a MIR122 promoter. In certain embodiments, the vectors provided herein comprise a VMD2 promoter.

In certain embodiments, the promoter is an inducible promoter. In certain embodiments the promoter is a hypoxia-inducible promoter. In certain embodiments, the promoter comprises a hypoxia-inducible factor (HIF) binding site. In certain embodiments, the promoter comprises a HIF-1α binding site. In certain embodiments, the promoter comprises a HIF-2α binding site. In certain embodiments, the HIF binding site comprises an RCGTG motif. For details regarding the location and sequence of HIF binding sites, see, e.g., Schödel, et al., Blood, 2011, 117(23):e207-e217, which is incorporated by reference herein in its entirety. In certain embodiments, the promoter comprises a binding site for a hypoxia induced transcription factor other than a HIF transcription factor. In certain embodiments, the viral vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety. In specific embodiments, the hypoxia-inducible promoter is the human N-WASP promoter, see, for example, Salvi, 2017, Biochemistry and Biophysics Reports 9:13-21 (incorporated by reference for the teaching of the N-WASP promoter) or is the hypoxia-induced promoter of human Epo, see, Tsuchiya et al., 1993, J. Biochem. 113:395-400 (incorporated by reference for the disclosure of the Epo hypoxia-inducible promoter). In other embodiments, the promoter is a drug inducible promoter, for example, a promoter that is induced by administration of rapamycin or analogs thereof. See, for example, the disclosure of rapamycin inducible promoters in PCT publications WO94/18317, WO 96/20951, WO 96/41865, WO 99/10508, WO 99/10510, WO 99/36553, and WO 99/41258, and U.S. Pat. No. 7,067,526, which are hereby incorporated by reference in their entireties for the disclosure of drug inducible promoters.

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron or a chimeric intron. In certain embodiments, the viral vectors provided herein comprise a polyadenylation sequence.

5.2.4 Signal Peptides

In certain embodiments, the vectors provided herein comprise components that modulate protein delivery. In certain embodiments, the viral vectors provided herein comprise nucleotide sequences encoding one or more signal peptides that are fused to the VEGF-trap fusion protein upon expression. Signal peptides may also be referred to herein as “leader sequences” or “leader peptides”. In certain embodiments, the signal peptides allow for the transgene product (e.g., the VEGF-Trap) to achieve the proper packaging (e.g. glycosylation) in the cell. In certain embodiments, the signal peptides allow for the transgene product (e.g., VEGF-Trap) to achieve the proper localization in the cell. In certain embodiments, the signal peptides allow for the transgene product (e.g., the VEGF-Trap) to achieve secretion from the cell.

There are two approaches to selecting signal peptides—either choosing a signal peptide from a protein homologous to the one being expressed or from a protein expressed in the cell type where the protein is to be expressed, processed and secreted. Signal peptides may be selected from appropriate proteins expressed in different species. The signal sequence of an abundantly expressed protein may be preferred. However, signal peptides may have some biological function after cleavage, “post-targeting” functions, so care should be taken to avoid signal peptides that may have such post-targeting function. Accordingly, the transgenes described herein may have signal peptides from human Flt-1 or KDR or related proteins or from proteins expressed in retinal or liver cells.

Aflibercept is expressed with the Flt-1 leader sequence and thus, transgenes are provided herein that have the Flt-1 leader sequence: MVSYWDTGVLLCALLSCLLLTGSSSG (SEQ ID NO: 36) (See FIG. 1). In alternative embodiments, the signal sequence is the KDR signal sequence, MQSKVLLAVALWLCVETRA (SEQ ID NO: 37). Alternatively and in preferred embodiments, the leader sequence used may be MYRMQLLLLI ALSLALVTNS (SEQ ID NO: 38) or MRMQLLLLIALSLALVTNS (SEQ ID NO: 39) (see FIGS. 2, 3 and 4). Examples of signal peptides to be used in connection with the vectors and transgenes provided herein, particularly for expression in retinal cells may be found, for example, in Table 3. See also, e.g., Stern et al., 2007, Trends Cell. Mol. Biol., 2:1-17 and Dalton & Barton, 2014, Protein Sci, 23: 517-525, each of which is incorporated by reference herein in its entirety for the signal peptides that can be used.

TABLE 3
Signal Sequences for Retinal Cell Secretion

| | | SEQ |
|---|---|---|
| Retinal Cell Protein | | ID |
| Signal Peptide | Sequence | NO: |
| VEGF-A signal peptide | MNFLLSWVHWSLALLLYLH | 59 |
| | HAKWSQA | |
| Fibulin-1 signal peptide | MERAAPSRRVPLPLLLLGG | 60 |
| | LALLAAGVDA | |
| Vitronectin signal | MAPLRPLLILALLAWVALA | 61 |
| peptide | | |
| Complement Factor H | MRLLAKIICLMLWAICVA | 62 |
| signal peptide | | |
| Opticin signal peptide | MRLLAFLSLLALVLQETGT | 63 |
| Albumin signal peptide | MKWVTFISLLFLFSSAYS | 64 |
| Chymotrypsinogen signal | MAFLWLLSCWALLGTTFG | 65 |
| peptide | | |
| Interleukin-2 signal | MYRMQLLSCIALILALVTN | 66 |
| peptide | S | |
| Trypsinogen-2 signal | MNLLLILTFVAAAVA | 67 |
| peptide | | |

Alternatively, for transgene products being expressed and secreted from liver cells, one of the signal sequences in Table 4 may be used.

TABLE 4
Signal Sequences for Secretion from Liver Cells

| Liver Cell Protein | | SEQ |
|---|---|---|
| Signal Peptide | Sequence | ID NO: |
| Human Serum albumin | MKWVTFISLLFLFSSAYS | 97 |
| Human α-1 Antitrypsin | MPSSVSWGILLLAGLCCL | 68 |
| (SERPINA1) | VPVSLA | |
| Human Apolipoprotein | MKAAVLTLAVLFLTGSQA | 69 |
| A-1 | | |
| Human Apolipoprotein | MKLLAATVLLLTICSLEG | 70 |
| A-2 | | |
| Human Apolipoprotein | MDPPRPALLALLALPALL | 71 |
| B-100 | LLLLAGARA | |
| Human Coagulation | MQRVNMIMAESPGLITIC | 72 |
| Factor IX | LLGYLLSAEC | |
| Human Complement | MGPLMVLFCLLFLYPGLA | 73 |
| C2 | DS | |
| Human Complement | MWLLVSVILISRISSVGG | 74 |
| Factor H-related | | |
| Protein 2 (CFHR2) | | |
| Human Complement | MLLLFSVILISWVSTVGG | 75 |
| Factor H-related | | |
| Protein 5 (CFHR5) | | |
| Human Fibrinogen | MFSMRIVCLVLSVVGTAWT | 76 |
| α-chain (FGA) | | |
| Human Fibrinogen | MKRMVSWSFHKLKTMKHL | 77 |
| β-chain (FGB) | LLLLLCVFLVKS | |
| Human Fibrinogen | MSWSLHPRNLILYFYALL | 78 |
| γ-chain (FGG) | FLSSTCVA | |
| Human α-2-HS- | MKSLVLLLCLAQLWGCHS | 79 |
| Glycoprotein (AHSG) | | |
| Human Hemopexin | MARVLGAPVALGLWSLCW | 80 |
| (HPX) | SLAIA | |
| Human Kininogen-1 | MKLITILFLCSRLLLSLT | 81 |
| Human Mannose- | MSLFPSLPLLLLSMVAASYS | 82 |
| binding protein C | | |
| (MBL2) | | |
| Human Plasminogen | MEHKEVVLLLLLFLKSGQG | 83 |
| (PLMN) | | |
| Human Prothrombin | MAHVRGLQLPGCLALAALC | 84 |
| (Coagulation Factor II) | SLVHS | |
| Human Secreted | MISRMEKMTMMMKILIMFA | 85 |
| Phosphoprotein 24 | LGMNYWSCSG | |
| Human Anti-thrombin- | MYSNVIGTVTSGKRKVYLL | 86 |
| III (SERPINC1) | SLLLIGFWDCVTC | |
| Human Serotransferrin | MRLAVGALLVCAVLGLCLA | 87 |
| (TF) | | |

5.2.5 Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half-life of the transgene. In certain embodiments, the UTRs are optimized for the stability of the mRNA of the transgene. In certain embodiments, the UTRs are optimized for the secondary structure of the mRNA of the transgene.

5.2.6 Polycistronic Messages—IRES and F2A Linkers

A single construct can be engineered to contain two “Fc-less” aflibercept transgenes separated by a cleavable linker or IRES so that two separate “Fc-less” aflibercept transgenes in one vector are expressed by the transduced cells. The Fc-less transgene may or may not contain the hinge region, and, for example, is the Fc-less transgene of FIG. 4. In certain embodiments, the viral vectors provided herein provide polycistronic (e.g., bicistronic) messages. For example, the viral construct can encode the two “Fc-less” aflibercept transgenes separated by an internal ribosome entry site (IRES) elements (for examples of the use of IRES elements to create bicistronic vectors see, e.g., Gurtu et al., 1996, Biochem. Biophys. Res. Comm. 229(1):295-8, which is herein incorporated by reference in its entirety). IRES elements bypass the ribosome scanning model and begin translation at internal sites. The use of IRES in AAV is described, for example, in Furling et al., 2001, Gene Ther 8(11): 854-73, which is herein incorporated by reference in its entirety. In certain embodiments, the bicistronic message is contained within a viral vector with a restraint on the size of the polynucleotide(s) therein. In certain embodiments, the bicistronic message is contained within an AAV virus-based vector (e.g., an AAV8-based vector).

In other embodiments, the viral vectors provided herein encode the two copies of the Fc-less transgene separated by a cleavable linker such as the self-cleaving furin/F2A (F/F2A) linkers (Fang et al., 2005, Nature Biotechnology 23: 584-590, and Fang, 2007, Mol Ther 15: 1153-9, each of which is incorporated by reference herein in its entirety). For example, a furin-F2A linker may be incorporated into an expression cassette to separate the two Fc-less VEGF-trap coding sequences, resulting in a construct with the structure:

Leader—Fc-less VEGF-Trap—Furin site—F2A site—Leader—Fc-less VEGF-Trap—PolyA.

The F2A site, with the amino acid sequence LLNFDLLKLAGDVESNPGP (SEQ ID NO: 88) is self-processing, resulting in “cleavage” between the final G and P amino acid residues. Additional linkers that could be used include but are not limited to:

(SEQ ID NO: 89)
T2A: (GSG)EGRGSLLTCGDVEENPGP
(SEQ ID NO: 90)
P2A: (GSG)ATNFSLLKQAGDVEENPGP
(SEQ ID NO: 91)
E2A: (GSG)QCTNYALLKLAGDVESNPGP
(SEQ ID NO: 92)
F2A: (GSG)VKQTLNFDLLKLAGDVESNPGP

A peptide bond is skipped when the ribosome encounters the F2A sequence in the open reading frame, resulting in the termination of translation, or continued translation of the downstream sequence. This self-processing sequence results in a string of additional amino acids at the end of the C-terminus of the first copy of the Fc-less VEGF-trap. However, such additional amino acids are then cleaved by host cell Furin at the furin sites, located immediately prior to the F2A site and after the first Fc-less VEGF-trap sequence, and further cleaved by carboxypeptidases. The resultant Fc-less VEGF-trap may have one, two, three, or more additional amino acids included at the C-terminus, or it may not have such additional amino acids, depending on the sequence of the Furin linker used and the carboxypeptidase that cleaves the linker in vivo (See, e.g., Fang et al., 17 Apr. 2005, Nature Biotechnol. Advance Online Publication; Fang et al., 2007, Molecular Therapy 15(6):1153-1159; Luke, 2012, Innovations in Biotechnology, Ch. 8, 161-186). Furin linkers that may be used comprise a series of four basic amino acids, for example, (SEQ ID NO: 93), RRRR (SEQ ID NO: 94), RRKR (SEQ ID NO: 95), or RKKR (SEQ ID NO: 96). Once this linker is cleaved by a carboxypeptidase, additional amino acids may remain, such that an additional zero, one, two, three or four amino acids may remain on the C-terminus of the heavy chain, for example, R, RR, RK, RKR, RRR, RRK, RKK, RKRR (SEQ ID NO: 93), RRRR (SEQ ID NO: 94), RRKR (SEQ ID NO: 95), or RKKR (SEQ ID NO: 96). In certain embodiments, one the linker is cleaved by a carboxypeptidase, no additional amino acids remain. In certain embodiments, 5%, 10%, 15%, or 20% of the VEGF-Trap population produced by the constructs described herein has one, two, three, or four amino acids remaining on the C-terminus after cleavage. In certain embodiments, the furin linker has the sequence R-X-K/R-R, such that the additional amino acids on the C-terminus of the VEGF-Trap are R, RX, RXK, RXR, RXKR, or RXRR, where X is any amino acid, for example, alanine (A). In certain embodiments, no additional amino acids may remain on the C-terminus of the VEGF-Trap.

In certain embodiments, an expression cassette described herein is contained within a viral vector with a restraint on the size of the polynucleotide(s) therein. In certain embodiments, the expression cassette is contained within an AAV virus-based vector (e.g., an AAV8-based vector).

5.2.7 Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan et al., 2005, J. Virol., 79(1):364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety).

In certain embodiments, the modified ITRs used to produce self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

5.2.8 Manufacture and Testing of Vectors

The viral vectors provided herein may be manufactured using host cells. The viral vectors provided herein may be manufactured using mammalian host cells, for example, A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (i.e., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

Alternatively, baculovirus expression systems in insect cells may be used to produce AAV vectors. For a review, see Aponte-Ubillus et al., 2018, Appl. Microbiol. Biotechnol. 102:1045-1054 which is incorporated by reference herein in its entirety for manufacturing techniques.

In vitro assays, e.g., cell culture assays, can be used to measure transgene expression from a vector described herein, thus indicating, e.g., potency of the vector. For example, the PER.C6° Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Alternatively, cell lines derived from liver or other cell types may be used, for example, but not limited, to HuH-7, HEK293, fibrosarcoma HT-1080, HKB-11, and CAP cells. Once expressed, characteristics of the expressed product (i.e., VEGF-Trap) can be determined, including determination of the glycosylation and tyrosine sulfation patterns associated with the VEGF-Trap. Glycosylation patterns and methods of determining the same are discussed herein. In addition, benefits resulting from glycosylation/sulfation of the cell-expressed VEGF-Trap can be determined using assays known in the art

5.2.9 Compositions

Compositions are described comprising a vector encoding a transgene described herein and a suitable carrier. A suitable carrier (e.g., for subretinal and/or intraretinal administration or for intravenous administration) would be readily selected by one of skill in the art.

### 5.3 Posttranslational Modifications: Glycosylation and Tyrosine Sulfation

In certain aspects, provided herein are VEGF-Trap proteins that contain human post-translational modifications. In one aspect, the VEGF-Trap proteins described herein contain the human post-translational modification of α2,6-sialylated glycans. In certain embodiments, the VEGF-Trap proteins only contain human post-translational modifications. In one embodiment, the VEGF-Trap proteins described herein do not contain the immunogenic non-human post-translational modifications of N-Glycolylneuraminic acid (Neu5Gc) and/or galactose-α-1,3-galactose (α-Gal) (or, do not contain levels detectable by assays that are standard in the art, for example, as described below). In another aspect, the VEGF-Trap proteins contain tyrosine (“Y”) sulfation sites. In one embodiment the tyrosine sites are sulfated in the Flt-1 Ig-like domain 2, the KDR Ig-like domain 3, and/or Fc domain of the fusion protein of the VEGF-Trap having the amino acid sequence of aflibercept. In other aspects, the VEGF-Trap proteins contain α2,6-sialylated glycans. In another aspect, the VEGF-Trap proteins contain α2,6-sialylated glycans and at least one sulfated tyrosine site. In other aspects, the VEGF-Trap proteins contain fully human post-translational modifications (VEGF-Trap^{HuPTM}). FIG. 1 highlights in yellow the amino acids of the VEGF-trap sequence of aflibercept that may be N-glycosylated and thus modified to have α2,6-sialylated glycans. Thus, provided are VEGF-Trap^{HuPTM} that have an α2,6-sialylated glycan at one, two, three, four or all five of positions 36, 68, 123, 196 and 282 of SEQ ID NO. 1 (highlighted in yellow on FIG. 1). Also provided are VEGF-Trap^{HuPTM} molecules that are sulfated at one, two, three or all four of the tyrosines at positions 11, 140, 263 and 281 of SEQ ID NO. 1 (highlighted in red in FIG. 1). In certain aspects, the post-translational modifications of the VEGF-Trap can be assessed by transducing an appropriate cell line, for example, PER.C6 or RPE cells (or, for non-retinal cells, HEK293, fibrosarcoma HT-1080, HKB-11, CAP, or HuH-7 cell lines) in culture with the transgene, which can result in production of said VEGF-Trap that is glycosylated and/or sulfated but does not contain detectable levels of NeuGc or α-Gal in said cell culture. Alternatively, or in addition, the production of said VEGF-Trap containing a tyrosine-sulfation can confirmed by transducing a PER.C6, RPE or non-retinal cell line such as HEK293, fibrosarcoma HT-1080, HKB-11, CAP, or HuH-7 with said recombinant nucleotide expression vector in cell culture.

In certain aspects, provided herein are methods for producing VEGF-Trap transgenes in human retinal cells as well as human retinal cells expressing the VEGF-Trap transgenes. In one embodiment, an expression vector encoding a VEGF-Trap, such as VEGF-Trap^{HuPTM}, can be administered to the subretinal space in the eye of a human subject wherein expression of said VEGF-Trap is α2,6-sialylated upon expression from said expression vector. In another embodiment, an expression vector encoding a VEGF-Trap is transfected into a human, immortalized retina-derived cell, and the VEGF-Trap transgene is expressed in the human, immortalized retina-derived cell and α2,6-sialylated upon expression. Human, immortalized retina-derived cells expressing α2,6-sialylated VEGF-Trap proteins are also provided herein. Additionally or alternatively, human retinal cells and/or human, immortalized retinal-derived cells can express a VEGF-Trap transgene containing at least one tyrosine-sulfation. Human retinal cell lines that can be used for such recombinant glycoprotein production include PER.C6 and RPE to name a few (e.g., see Dumont et al., 2015, Critical Rev in Biotech, 36(6):1110-1122 “Human cell lines for biopharmaceutical manufacturing: history, status, and future perspectives” which is incorporated by reference in its entirety for a review of the human cell lines that could be used for the recombinant production of the VEGF-Trap^{HuPTM} glycoprotein).

In certain aspects, provided herein are methods for producing VEGF-Trap transgenes in human liver cells as well as human liver cells expressing the VEGF-Trap transgenes. In one embodiment, an expression vector encoding a VEGF-Trap, such as VEGF-Trap^{HuPTM}, can be administered intravenously to a human subject wherein expression of said VEGF-Trap is α2,6-sialylated upon expression from said expression vector in liver cells of said human subject. In another embodiment, an expression vector encoding a VEGF-Trap is transfected into a human, immortalized liver-derived cell (or other immortalized human cell), and the VEGF-Trap transgene is expressed in the human, immortalized liver-derived (or other human immortalized) cell and α2,6-sialylated upon expression. Human, immortalized liver-derived (or other human immortalized) cells expressing α2,6-sialylated VEGF-Trap proteins are also provided herein. Additionally or alternatively, human liver cells and/or human, immortalized liver-derived cells can express a VEGF-Trap transgene containing at least one tyrosine-sulfation. Human liver cell lines that can be used for such recombinant glycoprotein production include HuH-7 cells, but may also include non-liver derived cells such as HEK293, fibrosarcoma HT-1080, HKB-11, CAP, and PER.C6 (e.g., see Dumont et al., supra).

The present invention provides gene therapy to deliver human-post-translationally modified VEGF-Trap (VEGF-Trap^{HuPTM}) proteins. It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment of the invention is to slow or arrest the progression of disease. In one particular embodiment of the present invention, the VEGF-Trap^{HuPTM} proteins have all of the human post-translational modifications and thus these proteins possess fully human glycosylation and sulfation. In other embodiments, only a 0.5 to 1% of the population of VEGF-Trap^{HuPTM} proteins are post-translationally modified and are therapeutically effective, or approximately 2%, or 1% to 5%, or 1% or 10% or greater than 10% of the molecules may be post-translationally modified and be therapeutically effective. In certain embodiments, the level of 2,6-sialylation and/or sulfation is significantly higher, such that up to 50%, 60%, 70%, 80%, 90% or even 100% of the molecules contains glycosylation and/or sulfation and are therapeutically effective. The goal of gene therapy treatment provided herein is to treat retinal neovascularization, and to maintain or improve vision with minimal intervention/invasive procedures or to treat, ameliorate or slow the progression of metastatic colon cancer. The presence of 2,6 sialylation can be tested by methods known in the art, see, for example, Rohrer, J. S., 2000, “Analyzing Sialic Acids Using High-Performance Anion-Exchange Chromatography with Pulsed Amperometric Detection.” Anal. Biochem. 283; 3-9.

In preferred embodiments, the VEGF-Trap^{HuPTM} proteins also do not contain detectable NeuGc and/or α-Gal. By “detectable NeuGc” or “detectable α-Gal” or “does not contain or does not have NeuGc or α-Gal” means herein that the VEGF-Trap^{HuPTM} does not contain NeuGc or α-Gal moieties detectable by standard assay methods known in the art. For example, NeuGc may be detected by HPLC according to Hara et al., 1989, “Highly Sensitive Determination of N-Acetyl- and N-Glycolylneuraminic Acids in Human Serum and Urine and Rat Serum by Reversed-Phase Liquid Chromatography with Fluorescence Detection.” J. Chromatogr., B: Biomed. 377, 111-119, which is hereby incorporated by reference for the method of detecting NeuGc. Alternatively, NeuGc may be detected by mass spectrometry. The α-Gal may be detected using an ELISA, see, for example, Galili et al., 1998, “A sensitive assay for measuring alpha-Gal epitope expression on cells by a monoclonal anti-Gal antibody.” Transplantation. 65(8):1129-32, or by mass spectrometry, see, for example, Ayoub et al., 2013, “Correct primary structure assessment and extensive glyco-profiling of cetuximab by a combination of intact, middle-up, middle-down and bottom-up ESI and MALDI mass spectrometry techniques.” Landes Bioscience. 5(5):699-710. See also the references cited in Platts-Mills et al., 2015, “Anaphylaxis to the Carbohydrate Side-Chain Alpha-gal” Immunol Allergy Clin North Am. 35(2): 247-260.

5.3.1 Glycosylation

Glycosylation can confer numerous benefits on the VEGF-Trap transgenes used in the compositions and methods described herein. Such benefits are unattainable by production of proteins in E. coli, because E. coli does not naturally possess components needed for N-glycosylation. Further, some benefits are unattainable through protein production in, e.g., CHO cells, because CHO cells lack components needed for addition of certain glycans (e.g., 2,6 sialic acid and bisecting GlcNAc) and because CHO cells can add glycans, e.g., Neu5Gc and α-Gal, not typical to and/or immunogenic in humans. See, e.g., Song et al., 2014, Anal. Chem. 86:5661-5666.

Human retinal cells are secretory cells that possess the cellular machinery for post-translational processing of secreted proteins—including glycosylation and tyrosine-O-sulfation, a robust process in retinal cells. (See, e.g., Wang et al., 2013, Analytical Biochem. 427: 20-28 and Adamis et al., 1993, BBRC 193: 631-638 reporting the production of glycoproteins by retinal cells; and Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells, each of which is incorporated by reference in its entirety for post-translational modifications made by human retinal cells).

Human hepatocytes are secretory cells that possess the cellular machinery for post-translational processing of secreted proteins—including glycosylation and tyrosine-O-sulfation. See, e.g. https://www.proteinatlas.org/humanproteome/liver for a proteomic identification of plasma proteins secreted by human liver; Clerc et al., 2016, Glycoconj 33:309-343 and Pompach et al., 2014, J Proteome Res. 13:5561-5569 for the spectrum of glycans on those secreted proteins; and E Mishiro, 2006, J Biochem 140:731-737 reporting that TPST-2 (which catalyzes tyrosine-O-sulfation) is more strongly expressed in liver than in other tissues, whereas TPST-1 was expressed in a comparable average level to other tissues, each of which is incorporated by reference in its entirety herein.

The VEGF-Trap, aflibercept, is a dimeric glycoprotein made in CHO cells with a protein molecular weight of 96.9 kilo Daltons (kDa). It contains approximately 15% glycosylation to give a total molecular weight of 115 kDa. All five putative N-glycosylation sites on each polypeptide chain predicted by the primary sequence can be occupied with carbohydrate and exhibit some degree of chain heterogeneity, including heterogeneity in terminal sialic acid residues.

Unlike CHO-cell products, such as aflibercept, glycosylation of VEGF-Trap^{HuPTM} by human retinal or liver cells, or other human cells, will result in the addition of glycans that can improve stability, half-life and reduce unwanted aggregation of the transgene product. (See, e.g., Bovenkamp et al., 2016, J. Immunol. 196: 1435-1441, for a review of the emerging importance of glycosylation in antibodies and Fabs). Significantly, the glycans that are added to VEGF-Trap^{HuPTM} of the invention are highly processed complex-type N-glycans that contain 2,6-sialic acid. Such glycans are not present in aflibercept which is made in CHO cells that do not have the 2,6-sialyltransferase required to make this post-translational modification, nor do CHO cells produce bisecting GlcNAc, although they do produce Neu5Gc (NGNA), which is immunogenic. See, e.g., Dumont et al., 2015, Critical Rev in Biotech, 36(6):1110-1122. Moreover, CHO cells can also produce an immunogenic glycan, the α-Gal antigen, which reacts with anti-α-Gal antibodies present in most individuals, which at high concentrations can trigger anaphylaxis. See, e.g., Bosques, 2010, Nat Biotech 28: 1153-1156. The human glycosylation pattern of the VEGF-Trap^{HuPTM} of the invention should reduce immunogenicity of the transgene product and improve safety and efficacy.

O-glycosylation comprises the addition of N-acetyl-galactosamine to serine or threonine residues by the enzyme. It has been demonstrated that amino acid residues present in the hinge region of antibodies can be O-glycosylated. In certain embodiments, the VEGF-Trap, used in the compositions and methods described herein, comprises all or a portion of the IgG Fc hinge region, and thus may be O-glycosylated when expressed in human retinal cells or liver cells. The possibility of O-glycosylation confers another advantage to the VEGF-Trap proteins provided herein, as compared to proteins produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098).

5.3.2 Tyrosine Sulfation

Tyrosine sulfation occurs at tyrosine (Y) residues with glutamate (E) or aspartate (D) within +5 to −5 position of Y, and where position −1 of Y is a neutral or acidic charged amino acid, but not a basic amino acid, e.g., arginine (R), lysine (K), or histidine (H) that abolishes sulfation. Accordingly, the compositions and methods described herein comprise use of VEGF-Trap proteins that comprise at least one tyrosine sulfation site, which when expressed in human retinal cells or liver cells or other human cells, can be tyrosine sulfated.

Importantly, tyrosine-sulfated proteins cannot be produced in E. coli, which naturally does not possess the enzymes required for tyrosine-sulfation. Further, CHO cells are deficient for tyrosine sulfation—they are not secretory cells and have a limited capacity for post-translational tyrosine-sulfation. See, e.g., Mikkelsen & Ezban, 1991, Biochemistry 30: 1533-1537. Advantageously, the methods provided herein call for expression of VEGF-Trap transgenes in retinal cells or liver cells, which are secretory and do have capacity for tyrosine sulfation. See Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells.

Tyrosine sulfation is advantageous for several reasons. For example, tyrosine-sulfation of the antigen-binding fragment of therapeutic antibodies against targets has been shown to dramatically increase avidity for antigen and activity. See, e.g., Loos et al., 2015, PNAS 112: 12675-12680, and Choe et al., 2003, Cell 114: 161-170. Assays for detection tyrosine sulfation are known in the art. See, e.g., Yang et al., 2015, Molecules 20:2138-2164.

In addition to the glycosylation sites, VEGF-Traps such as aflibercept may contain tyrosine (“Y”) sulfation sites; see FIG. 1 in which the sulfation sites are highlighted in red and identifies tyrosine-O-sulfation sites in the Flt-1 Ig-like domain 2, the KDR Ig-like domain 3, and Fc domain of aflibercept at positions 11 (Flt-1 Ig-like domain), 140 (KDR Ig-like domain), 263 and 281 (IgG1 Fc domain) of SEQ ID NO: 1. (See, e.g., Yang et al., 2015, Molecules 20:2138-2164, esp. at p. 2154 which is incorporated by reference in its entirety for the analysis of amino acids surrounding tyrosine residues subjected to protein tyrosine sulfation).

### 5.4. Gene Therapy Protocol

Methods are described for the administration of a therapeutically effective amount of a transgene construct to human subjects having an ocular disease caused by increased neovascularization. More particularly, methods for administration of a therapeutically effective amount of a transgene construct to patients having nAMD, diabetic retinopathy, DME, RVO, pathologic myopia, or polypoidal choroidal vasculopathy, described. In specific, embodiments, the vector is administered subretinally (a surgical procedure performed by trained retinal surgeons that involves a partial vitrectomy with the subject under local anesthesia, and injection of the gene therapy into the retina; see, e.g., Campochiaro et al., 2016, Hum Gen Ther Sep 26 epub:doi: 10.1089/hum.2016.117, which is incorporated by reference herein in its entirety), or intravitreally, or suprachoroidally such as by microinjection or microcannulation. (See, e.g., Patel et al., 2012, Invest Ophth & Vis Sci 53:4433-4441; Patel et al., 2011, Pharm Res 28:166-176; Olsen, 2006, Am J Ophth 142:777-787 each of which is incorporated by reference in its entirety). In particular embodiments, such methods for subretinal and/or intraretinal administration of a therapeutically effective amount of a transgene construct result in expression of the transgene in one or more of human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells to deliver the VEGF-Trap^{HuPTM} to the retina.

Methods are described for the administration of a therapeutically effective amount of a transgene construct to human subjects having cancer, particularly metastatic colon cancer to create a depot of cells in the liver of the human subject that express the VEGF-Trap^{HuPTM} for delivery to the colon cancer cells and/or the tissue surrounding the colon cancer cells. In particular, methods provide for intravenous administration or direct administration to the liver through hepatic blood flow, such as, via the suprahepatic veins or hepatic artery. Such methods result in expression of the transgene in liver cells to deliver the VEGF-Trap^{HuPTM} to cancer cells and/or the neovascularized tissue surrounding the cancer cells.

5.4.1 Target Patient Populations

In certain embodiments, the methods provided herein are for the administration to patients diagnosed with an ocular disease caused by increased neovascularization.

In certain embodiments, the methods provided herein are for the administration to patients diagnosed with severe AMD. In certain embodiments, the methods provided herein are for the administration to patients diagnosed with attenuated AMD.

In certain embodiments, the methods provided herein are for the administration to patients diagnosed with severe wet AMD. In certain embodiments, the methods provided herein are for the administration to patients diagnosed with attenuated wet AMD.

In certain embodiments, the methods provided herein are for the administration to patients diagnosed with severe diabetic retinopathy. In certain embodiments, the methods provided herein are for the administration to patients diagnosed with attenuated diabetic retinopathy. In certain embodiments, the methods provided herein are for the administration to patients diagnosed with diabetic retinopathy associated with diabetic macular edema (DME).

In certain embodiments, the methods provided herein are for the administration to patients diagnosed with severe diabetic retinopathy. In certain embodiments, the methods provided herein are for the administration to patients diagnosed with attenuated diabetic retinopathy.

In certain embodiments, the methods provided herein are for the administration to patients diagnosed with central retinal vein occlusion (RVO), macular edema following RVO, pathologic myopia or polypoidal choroidal vasculopathy.

In certain embodiments, the methods provided herein are for the administration to patients diagnosed with AMD who have been identified as responsive to treatment with a VEGF-Trap fusion protein.

In certain embodiments, the methods provided herein are for the administration to patients diagnosed with AMD who have been identified as responsive to treatment with a aflibercept.

In certain embodiments, the methods provided herein are for the administration to patients diagnosed with AMD who have been identified as responsive to treatment with a VEGF-Trap fusion protein, such as aflibercept, injected intravitreally prior to treatment with gene therapy.

In certain embodiments, the methods provided herein are for the administration to patients diagnosed with AMD who have been identified as responsive to treatment with a VEGF-Trap^{HuPTM} that has been produced by expression in immortalized human retinal cells injected intravitreally prior to treatment with gene therapy.

In certain embodiments, the methods provided herein are for the administration to patients diagnosed with AMD, diabetic retinopathy, DME, central retinal vein occlusion (RVO), pathologic myopia, polypoidal choroidal vasculopathy who have been identified as responsive to treatment with LUCENTIS® (ranibizumab), EYLEA® (aflibercept), and/or AVASTIN® (bevacizumab).

In certain embodiments, the methods provided herein are for the administration to patients diagnosed with cancer, particularly metastatic cancer. In certain embodiments, the methods provided herein are for the administration to patients diagnosed with metastatic colon cancer.

In certain embodiments, the methods provided herein are for the administration to patients diagnosed with metastatic cancer, particularly metastatic colon cancer, who have been identified as responsive to treatment with a VEGF-Trap fusion protein.

In certain embodiments, the methods provided herein are for the administration to patients diagnosed with metastatic cancer, particularly metastatic colon cancer, who have been identified as responsive to treatment with ziv-aflibercept.

In certain embodiments, the methods provided herein are for the administration to patients diagnosed with metastatic cancer, particularly metastatic colon cancer, who have been identified as responsive to treatment with a VEGF-Trap fusion protein, such as ziv-aflibercept, infused intravenously prior to treatment with gene therapy.

In certain embodiments, the methods provided herein are for the administration to patients diagnosed with metastatic cancer, particularly metastatic colon cancer, who have been identified as responsive to treatment with a VEGF-Trap^{HuPTM} that has been produced by expression in immortalized human cells infused intravenously prior to treatment with gene therapy.

In certain embodiments, the methods provided herein are for the administration to patients diagnosed with metastatic cancer, particularly metastatic colon cancer, who have been identified as responsive to treatment with ZALTRAP® (ziv-aflibercept), and/or AVASTIN® (bevacizumab), and/or STIVARGA® (regorafenib).

5.4.2 Dosage and Mode of Administration

Therapeutically effective doses of the recombinant vector should be delivered to the eye, e.g., to the subretinal space, or to the suprachoroidal space, or intravitreally in an injection volume ranging from 0.1 mL to 0.5 mL, preferably in 0.1 to 0.25 mL (100-250 μl). Doses that maintain a concentration of the transgene product detectable at a Cₘᵢₙ of at least about 0.33 μg/mL to about 1.32μg/mL in the vitreous humour, or about 0.11 μg/mL to about 0.44 μg/mL in the Aqueous humour (the anterior chamber of the eye) for three months are desired; thereafter, Vitreous Cₘᵢₙ concentrations of the transgene product ranging from about 1.70 to about 6.60 μg/mL and up to about 26.40 μg/mL, and/or Aqueous Cₘᵢₙ concentrations ranging from about 0.56 to about 2.20 μg/mL, and up to 8.80 μg/mL should be maintained. Vitreous humour concentrations can be estimated and/or monitored by measuring the patient's aqueous humour or serum concentrations of the transgene product. Alternatively, doses sufficient to achieve a reduction in free-VEGF plasma concentrations to about 10 pg/mL can be used. (E.g., see, Avery et al., 2017, Retina, the Journal of Retinal and Vitreous Diseases 0:1-12; and Avery et al., 2014, Br J Ophthalmol 98:1636-1641 each of which is incorporated by reference herein in its entirety).

For treatment of cancer, particularly metastatic colon cancer, therapeutically effective doses should be administered to the patient, preferably intravenously, such that plasma concentrations of the transgene are maintained, after two weeks or four weeks at levels at least the Cₘᵢₙ plasma concentrations of ziv-aflibercept when administered at a dose of 4 mg/kg every two weeks.

### 5.5 Biomarkers/Sampling/Monitoring Efficacy

Effects of the methods of treatment provided herein on visual deficits may be measured by BCVA (Best-Corrected Visual Acuity), intraocular pressure, slit lamp biomicroscopy, and/or indirect ophthalmoscopy.

Effects of the methods of treatment provided herein on physical changes to eye/retina may be measured by SD-OCT (SD-Optical Coherence Tomography).

Efficacy may be monitored as measured by electroretinography (ERG).

Effects of the methods of treatment provided herein may be monitored by measuring signs of vision loss, infection, inflammation and other safety events, including retinal detachment.

Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458).

Efficacy of treatment for cancer, particularly metastatic colon cancer, may be monitored by any means known in the art for evaluating the efficacy of an anti-cancer/anti-metastatic agent, such as a reduction in tumor size, reduction in number and/or size of metastases, increase in overall survival, progression free survival, response rate, incidence of stable disease,

### 5.6 Combination Therapies

The methods of treatment provided herein may be combined with one or more additional therapies. In one aspect, the methods of treatment provided herein are administered with laser photocoagulation. In one aspect, the methods of treatment provided herein are administered with photodynamic therapy with verteporfin or intraocular steroids.

In one aspect, the methods of treatment provided herein are administered with intravitreal (IVT) injections with anti-VEGF agents, including but not limited to VEGF-Trap^{HuPTM} produced in human cell lines (Dumont et al., 2015, supra), or other anti-VEGF agents such as aflibercept, ranibizumab, bevacizumab, or pegaptanib. Combinations of delivery of the VEGF-TrapHuPTM to the eye/retina accompanied by delivery of other available treatments are described herein. The additional treatments may be administered before, concurrently or subsequent to the gene therapy treatment. Available treatments for nAMD, diabetic retinopathy, DME, cRVO, pathologic myopia, or polypoidal choroidal vasculopathy, that could be combined with the gene therapy of the invention include but are not limited to laser photocoagulation, photodynamic therapy with verteporfin, and intravitreal (IVT) injections with anti-VEGF agents, including but not limited to aflibercept, ranibizumab, bevacizumab, or pegaptanib, as well as treatment with intravitreal steroids to reduce inflammation. Available treatments for metastatic colon cancer, that could be combined with the gene therapy methods include but are not limited to surgery and/or chemotherapy agents useful for treatment of cancer, particularly, metastatic colon cancer. In particular embodiments, the gene therapy methods are administered with the regimens used for treatment of metastatic colon cancer, specifically, 5-fluorouracil, leucovorin, irinotecan (FOLFIRI) or folinic acid (also called leucovorin, FA or calcium folinate), 5-fluorouracil, and/or oxaliplatin (FOLFOX), and intravenous administration with anti-VEGF agents, including but not limited to ziv-aflibercept, ranibizumab, bevacizumab, pegaptanib or regorafenib.

The methods of treatment provided herein may be combined with one or more additional therapies. In one aspect, the methods of treatment for ocular disease provided herein are administered with laser photocoagulation. In one aspect, the methods of treatment for ocular disease provided herein are administered with photodynamic therapy with verteporfin or intraocular steroids.

In one aspect, the methods of treatment provided herein are administered with intravitreal (IVT) injections or intravenous administration with anti-VEGF agents, including but not limited to VEGF-Trap^{HuPTM} produced in human cell lines (Dumont et al., 2015, supra), or other anti-VEGF agents such as aflibercept, ranibizumab, bevacizumab, pegaptanib or regorafenib.

The additional therapies may be administered before, concurrently or subsequent to the gene therapy treatment.

The efficacy of the gene therapy treatment may be indicated by the elimination of or reduction in the number of rescue treatments using standard of care, for example, intravitreal injections with anti-VEGF agents, including but not limited to VEGF-Trap^{HuPTM} produced in human cell lines or other anti-VEGF agents such as aflibercept, ranibizumab, bevacizumab, or pegaptanib.

### EXAMPLES

### 6.1 Example 1

### Aflibercept cDNA (and Codon Optimized)

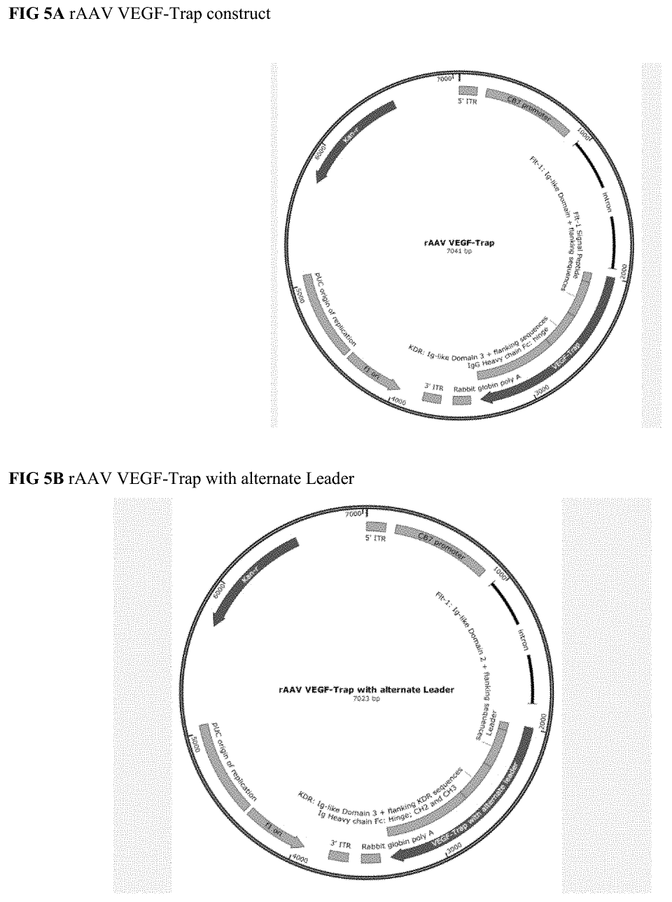

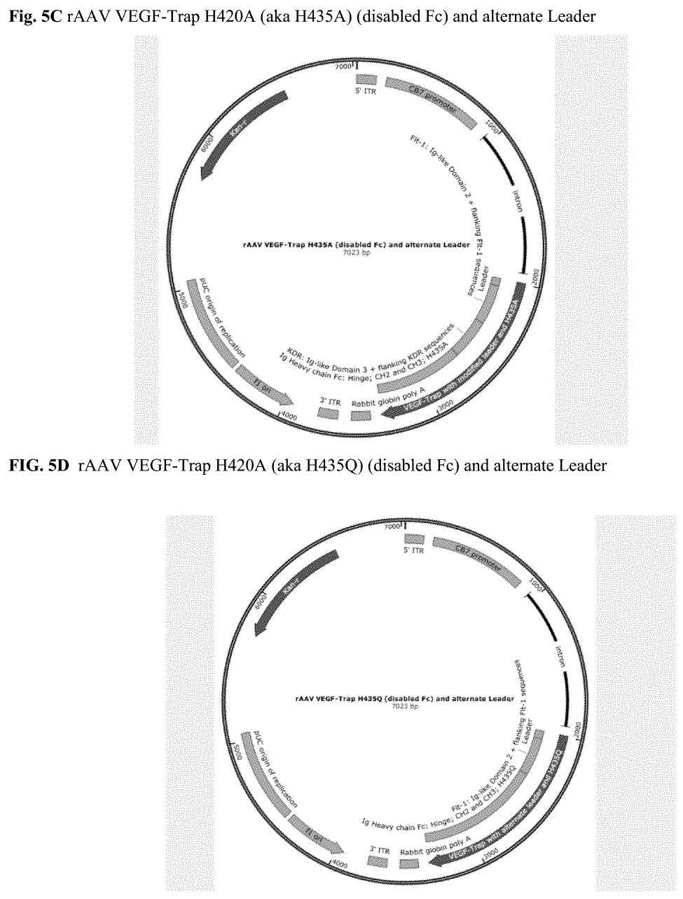

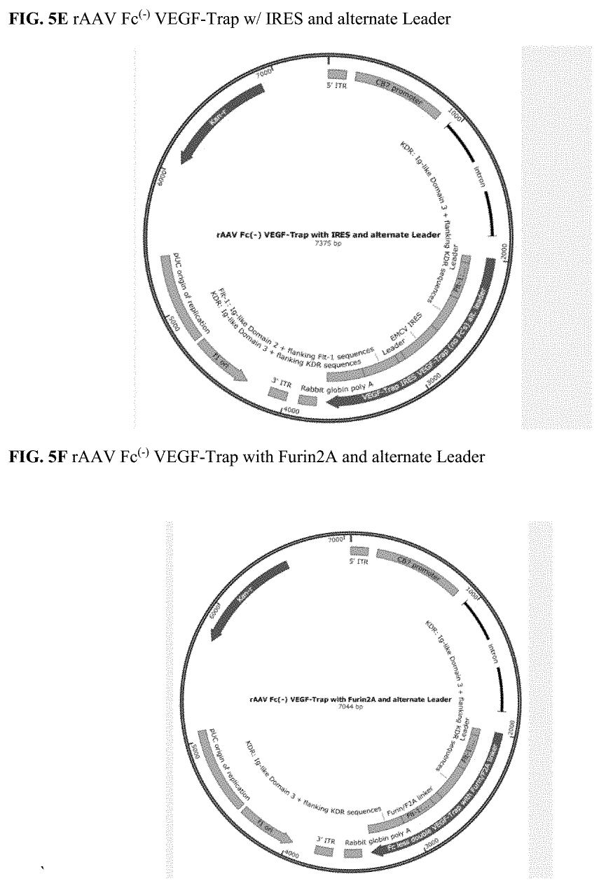

An aflibercept cDNA-based vector is constructed comprising a transgene comprising a nucleotide sequence encoding the aflibercept sequence of SEQ ID NO: 1 with the Flt-1 signal sequence MVSYWDTGVLLCALLSCLLLTGSS_SG (SEQ ID NO: 36) (see FIG. 1). The transgene sequence is codon optimized for expression in human cells (e.g., the nucleotide sequence of SEQ ID NO: 2 or SEQ ID NO: 3). The vector additionally comprises a ubiquitously active, constitutive promoter such as CB7, or optionally, a hypoxia-inducible promoter. A map of the vector is provided in FIG. 5A.

### 6.2 Example 2

### Aflibercept with Alternate Leader

An aflibercept cDNA-based vector is constructed comprising a transgene comprising a nucleotide sequence encoding the aflibercept sequence of SEQ ID NO: 1 with leader sequence MYRMQLLLLIALSLALVTNS (SEQ ID NO: 38) (amino acid sequence provided in FIG. 2). The transgene sequence is codon optimized for expression in human cells (for example, the aflibercept amino acid sequence, minus the leader sequence of SEQ ID NO: 2 or SEQ ID NO: 3) The vector additionally comprises a ubiquitously active, constitutive promoter such as CB7, or optionally, a hypoxia-inducible promoter. A map of the vector is provided in FIG. 5B.

### 6.3 Example 3

### Aflibercept with “Disabled Fc” (H420A; H420Q)

An aflibercept cDNA-based vector is constructed comprising a transgene comprising a nucleotide sequence encoding the aflibercept sequence of SEQ ID NO: 1 except that the histidine at position 420 (corresponding to position 435 in the usual numbering of the Fc) is replaced with either an alanine (A) or a glutamine (Q) and encoding an N-terminal leader sequence MYRMQLLLLIALSLALVTNS (SEQ ID NO: 38) (as set forth in FIG. 3). The transgene sequence is codon optimized for expression in human cells. The vector additionally comprises a ubiquitously active, constitutive promoter such as CB7, or optionally, a hypoxia-inducible promoter. Maps of the vector is provided in FIGS. 5C (alanine substitution) and 5D (glutamine substitution).

### 6.4 Example 4

### Fc⁽⁻⁾ Aflibercept

An aflibercept cDNA-based vector is constructed comprising a transgene comprising a nucleotide sequence encoding an Fc-less form of the aflibercept sequence of SEQ ID NO: 1 in which the transgene encodes a VEGF-trap with the amino acid sequence of positions 1 to 204 of SEQ ID NO:1 (deleted for the terminal lysine of the KDR sequence and the IgG1 Fc domain) or a VEGF-trap with the amino acid sequence of positions 1 to 205 of SEQ ID NO:1 (having the terminal lysine of the KDR sequence but deleted for the IgG1 Fc domain), or a VEGF-trap with the amino acid sequence of positions 1 to 216 (having a portion of the hinge region of the IgG1 Fc domain), or a VEGF-trap with the amino acid sequence of positions 1 to 222 of SEQ ID NO: 1 (having the hinge region of IgG1 Fc domain), or a VEGF-Trap with the amino acid sequence of positions 1 to 227 (se FIG. 4). The construct also encodes at the N-terminus of the VEGF-trap a leader sequence MYRMQLLLLIALSLALVTNS (SEQ ID NO: 38) (amino acid sequence provided in FIG. 2). The transgene sequence is codon optimized for expression in human cells. The vector additionally comprises a ubiquitously active, constitutive promoter such as CB7, or optionally, a hypoxia-inducible promoter.

### 6.5 Example 5

### Fc(−)Aflibercept Double Constructs

A tandem aflibercept cDNA-based vector is constructed comprising a transgene comprising two nucleotide sequences encoding an Fc-less form of the aflibercept sequence of SEQ ID NO: 1 in which the transgene comprises two (preferably identical) nucleotide sequences each encoding a VEGF-trap with the amino acid sequence of positions 1 to 204 of SEQ ID NO:1 (deleted for the terminal lysine of the KDR sequence and the IgG1 Fc domain) or a VEGF-trap with the amino acid sequence of positions 1 to 205 of SEQ ID NO:1 (having the terminal lysine of the KDR sequence but deleted for the IgG1 Fc domain), or a VEGF-trap with the amino acid sequence of positions 1 to 216 (having a portion of the hinge region of the IgG1 Fc domain), or a VEGF-trap with the amino acid sequence of positions 1 to 222 of SEQ ID NO: 1 (having the hinge region of IgG1 Fc domain), or a VEGF-Trap with the amino acid sequence of positions 1 to 227 of SEQ ID NO: 1. The construct also encodes at the N-terminus of each of the VEGF-trap sequences a leader sequence of Table 3 for retinal cell expression or table 4 for liver cell expression. The nucleotide sequences encoding the two VEGF-trap encoding sequences are separated by IRES elements or 2A cleavage sites to create a bicistronic vector. The vector additionally comprises a ubiquitously active, constitutive promoter such as CB7, or optionally, a hypoxia-inducible promoter. Exemplary vectors are shown in FIGS. 5E and 5F.

### Equivalents

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

1. An expression construct comprising an expression cassette flanked by AAV inverted terminal repeats (ITRs) wherein the expression cassette comprises a transgene encoding a VEGF-TrapHuPTM operably linked to one or more regulatory sequences that control expression of the transgene in human retinal cells or human liver cells, wherein the transgene encodes a leader sequence operable in human retinal cells or human liver cells and a VEGF-TrapHuPTM, wherein the VEGF-TrapHuPTM comprises an amino acid sequence having amino acid residues 1 to 204 of SEQ ID NO: 1.

2. The expression construct of claim 1 wherein the VEGF-TrapHuPTM comprises an amino acid sequence having amino acid residues 1 to 205 of SEQ ID NO: 1 linked at the C terminus to an IgG1, IgG2, or IgG4 Fc region comprising at least a partial hinge region at the N-terminus of the Fc region.

3. The expression construct of claim 2, wherein the Fc region comprises a full hinge region.

4. The expression construct of claim 2, wherein one or more of the cysteine residues within the hinge region is substituted with a serine.

5. The expression construct of claim 2, wherein the Fc region has one or more amino acid substitutions which reduce FcRn binding compared to the Fc region without the amino acid substitutions.

6. The expression construct of claim 1 wherein the VEGF-TrapHuPTM comprises an amino acid sequence having amino acid residues 1 to 205 of SEQ ID NO: 1 linked at the C terminus to an Ig-like domain of Flt-1 or KDR.

7. The expression construct of claim 1, wherein the expression construct comprises a second VEGF-TrapHuPTM comprising an amino acid sequence having amino acid residues 1 to 204 of SEQ ID NO: 1.

8. The expression construct of claim 1 wherein the VEGF-TrapHuPTM has an amino acid sequence selected from
i. the amino acid sequence of SEQ ID NO: 1 (FIG. 1),
ii. the amino acid sequence of SEQ ID NO: 1 with an alanine substitution at position 238 and/or 295 and/or an alanine or glutamine substitution at position 420;
iii. the amino acid sequence of SEQ ID NO: 1 with an alanine or glutamine substitution at position 420 (FIG. 3);
iv. the amino acid sequence of amino acid residues 1 to 205 of SEQ ID NO: 1 and optionally linked to the C-terminus a sequence selected from SEQ ID Nos: 46 to 48 (FIG. 4);
v. the amino acid sequence consisting of residues 1 to 204 of SEQ ID NO: 1;
vi. the amino acid sequence of amino acid sequence residues 1 to 205 of SEQ ID NO: 1 linked at the C terminus to one of the amino acid sequences of SEQ ID NOs: 19, 20, 49, 50, 51, 52, 53, or 54 (FIG. 7C-7H); and
vii. the amino acid sequence of amino acid sequence residues 1 to 205 of SEQ ID NO: 1 linked at the C terminus to either SEQ ID NO: 55 or 56. (FIG. 8C/8D)

9. The expression construct of clam 1, wherein the leader sequence is one of SEQ ID Nos:
36 to 39 or 59 to 67. (retinal cells)

10. The expression construct of claim 1, wherein the leader sequence is one of SEQ ID Nos: 68 to 87 or 97. (liver cells)

11. The expression construct of claim 1, wherein at least one of the regulatory sequences is a constitutive promoter.

12. The expression construct of claim 1, wherein the one or more regulatory sequences are a CB7 promoter, a chicken β-actin intron and a rabbit β-globin poly A signal.

13. The expression construct of claim 1, wherein at least one of the regulatory sequences is an inducible promoter, optionally a hypoxia-inducible promoter or a rapamycin inducible promoter.

14. An adeno-associated virus (AAV) vector comprising a viral capsid that is at least 95% identical to the amino acid sequence of an AAV8 capsid (SEQ ID NO: 11) or AAV2 capsid (SEQ ID NO: 5) or is a variant of AAV8 or AAV2, and a viral genome comprising an expression construct of claim 1.

15. The AAV vector of claim 14, wherein the viral capsid is AAV.7m8.

16. A pharmaceutical composition for ocular administration comprising an AAV vector comprising:
a viral capsid that is at least 95% identical to the amino acid sequence of an AAV8 capsid (SEQ ID NO: 11) or AAV2 capsid (SEQ ID NO: 5) or is a variant of AAV8 or AAV2; and
a viral genome comprising an expression construct of claim 1;
wherein said AAV vector is formulated for subretinal, intravitreal or suprachororidal administration to the eye of said subject.

17. The pharmaceutical composition of claim 16, wherein the viral capsid is AAV.7m8.

18. A pharmaceutical composition for intravenous administration comprising an AAV vector comprising:
a viral capsid that is at least 95% identical to the amino acid sequence of an AAV8 capsid (SEQ ID NO: 11) or is a variant of AAV8; and
a viral genome comprising an expression construct of claim 1;
wherein said AAV vector is formulated for intravenous administration to said subject.

19. A method of treating a human subject diagnosed with metastatic colon cancer or an eye related disorder selected from neovascular age-related macular degeneration (nAMD), diabetic retinopathy, diabetic macular edema (DME), central retinal vein occlusion (RVO), pathologic myopia, or polypoidal choroidal vasculopathy, said method comprising delivering to the retina of said human subject with the eye-related disorder or to the cancer cells or neovascularized tissue around said cancer cells of said human subject with metastatic colon cancer, a therapeutically effective amount of VEGF-TrapHuPTM produced by human liver cells or human retinal cells selected from human photoreceptor cells (cone cells, rod cells); horizontal cells; bipolar cells; amacrine cells; retina ganglion cells (midget cell, parasol cell, bistratified cell, giant retina ganglion cell, photosensitive ganglion cell, and mullerglia); and retinal pigment epithelial cells, wherein the VEGF-TrapHuPTM comprises an amino acid sequence having amino acid residues 1 to 204 of SEQ ID NO: 1.

20. A method of treating a human subject diagnosed metastatic colon cancer or an eye related disorder selected from neovascular age-related macular degeneration (nAMD), diabetic retinopathy, diabetic macular edema (DME), central retinal vein occlusion (RVO), pathologic myopia, or polypoidal choroidal vasculopathy, said method comprising delivering to the retina of said human subject with the eye-related disorder or to the cancer cells or neovascularized tissue around said cancer cells of said human subject with metastatic colon cancer, a therapeutically effective amount of a VEGF-TrapHuPTM containing an α2,6-sialylated glycan and/or a tyrosine sulfation, wherein the VEGF-TrapHuPTM comprises an amino acid sequence having amino acid residues 1 to 204 of SEQ ID NO: 1.

21. The method of claim 20, wherein the VEGF-TrapHuPTM expressed does not contain detectable NeuGc or α-Gal.

22. A method of treating a human subject diagnosed with metastatic colon cancer or an eye related disorder selected from neovascular age-related macular degeneration (nAMD), diabetic retinopathy, diabetic macular edema (DME), central retinal vein occlusion (RVO), pathologic myopia, or polypoidal choroidal vasculopathy, said method comprising: administering to the liver of said human subject with metastatic colon cancer and to the the subretinal space in the eye of said human subject with the eye-related disorder, a therapeutically effective amount of a recombinant nucleotide expression vector comprising an expression construct of claims 1, wherein VEGF-TrapHuPTM expressed in the liver contains a α2,6-sialylated glycan or tyrosine-sulfation.

23. The method of claim 22, wherein the VEGF-TrapHuPTM expressed does not contain detectable NeuGc or α-Gal.

24. The method of claim 22, wherein the recombinant nucleotide expression vector is an AAV8 viral vector or an AAV2 viral vector or an AAV viral vector that is a variant of AVV2 or AAV8.

25. The method of claim 24, wherein the recombinant nucleotide expression vector is an AAV.7m8 viral vector.

26. A method of manufacturing an AAV2 or AAV8 viral vector comprising a VEGF-Trap transgene, said method comprising culturing host cells under conditions appropriate for production of the AAV2 or AAV8 viral vector, wherein the host cells are stably transformed with a nucleic acid vector comprise an expression construct of claim 1 comprising nucleotide sequences encoding the AAV2 or AAV8 replication and capsid proteins or variants thereof; and recovering the AAV2 or AAV8 viral vector produced by the host cell.

27. The method of claim 26, wherein the viral vector comprises nucleotide sequences encoding the AAV.7m8 replication and capsid proteins.

28. A method of producing recombinant AAVs comprising:
(a) culturing a host cell containing:
(i) an artificial genome comprising an expression construct of claim 1;
(ii) a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans;
(iii) sufficient adenovirus helper functions to permit replication and packaging of the artificial genome by the AAV capsid proteins; and
(b) recovering recombinant AAV encapsidating the artificial genome from the cell culture.


SEQUENCE LISTING (97 sequences)

SEQ ID NO: 1 - protein, 431 aa, Artificial Sequence
  source: /note=“Description of Artificial Sequence Synthetic polypeptide”
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - DNA, 1353 bp, Artificial Sequence
  source: /note=“Description of Artificial Sequence Synthetic polynucleotide”
atgtacagaatgcagctgctgctgctgatcgccctgagcctggccctggtgaccaacagcagcgacaccggcagacccttcgtggagatgtacagcgagatccccgagatcatccacatgaccgagggcagagagctggtgatcccctgcagagtgaccagccccaacatcaccgtgaccctgaagaagttccccctggacaccctgatccccgacggcaagagaatcatctgggacagcagaaagggcttcatcatcagcaacgccacctacaaggagatcggcctgctgacctgcgaggccaccgtgaacggccacctgtacaagaccaactacctgacccacagacagaccaacaccatcatcgacgtggtgctgagccccagccacggcatcgagctgagcgtgggcgagaagctggtgctgaactgcaccgccagaaccgagctgaacgtgggcatcgacttcaactgggagtaccccagcagcaagcaccagcacaagaagctggtgaacagagacctgaagacccagagcggcagcgagatgaagaagttcctgagcaccctgaccatcgacggcgtgaccagaagcgaccagggcctgtacacctgcgccgccagcagcggcctgatgaccaagaagaacagcaccttcgtgagagtgcacgagaaggacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggc

SEQ ID NO: 3 - DNA, 1353 bp, Artificial Sequence
  source: /note=“Description of Artificial Sequence Synthetic polynucleotide”
  modified_base (9)..(9): a, c, t, g, unknown or other
  modified_base (18)..(18): a, c, t, g, unknown or other
  modified_base (21)..(21): a, c, t, g, unknown or other
  modified_base (24)..(24): a, c, t, g, unknown or other
  modified_base (27)..(27): a, c, t, g, unknown or other
  modified_base (33)..(33): a, c, t, g, unknown or other
  modified_base (36)..(36): a, c, t, g, unknown or other
  modified_base (39)..(39): a, c, t, g, unknown or other
  modified_base (42)..(42): a, c, t, g, unknown or other
  modified_base (45)..(45): a, c, t, g, unknown or other
  modified_base (48)..(48): a, c, t, g, unknown or other
  modified_base (51)..(51): a, c, t, g, unknown or other
  modified_base (54)..(54): a, c, t, g, unknown or other
  modified_base (60)..(60): a, c, t, g, unknown or other
  modified_base (63)..(63): a, c, t, g, unknown or other
  modified_base (69)..(69): a, c, t, g, unknown or other
  modified_base (72)..(72): a, c, t, g, unknown or other
  modified_base (75)..(75): a, c, t, g, unknown or other
  modified_base (78)..(78): a, c, t, g, unknown or other
  modified_base (84)..(84): a, c, t, g, unknown or other
  modified_base (96)..(96): a, c, t, g, unknown or other
  modified_base (105)..(105): a, c, t, g, unknown or other
  modified_base (123)..(123): a, c, t, g, unknown or other
  modified_base (129)..(129): a, c, t, g, unknown or other
  modified_base (132)..(132): a, c, t, g, unknown or other
  modified_base (138)..(138): a, c, t, g, unknown or other
  modified_base (141)..(141): a, c, t, g, unknown or other
  modified_base (147)..(147): a, c, t, g, unknown or other
  modified_base (153)..(153): a, c, t, g, unknown or other
  modified_base (156)..(156): a, c, t, g, unknown or other
  modified_base (159)..(159): a, c, t, g, unknown or other
  modified_base (162)..(162): a, c, t, g, unknown or other
  modified_base (165)..(165): a, c, t, g, unknown or other
  modified_base (174)..(174): a, c, t, g, unknown or other
  modified_base (177)..(177): a, c, t, g, unknown or other
  modified_base (180)..(180): a, c, t, g, unknown or other
  modified_base (183)..(183): a, c, t, g, unknown or other
  modified_base (195)..(195): a, c, t, g, unknown or other
  modified_base (198)..(198): a, c, t, g, unknown or other
  modified_base (204)..(204): a, c, t, g, unknown or other
  modified_base (207)..(207): a, c, t, g, unknown or other
  modified_base (213)..(213): a, c, t, g, unknown or other
  modified_base (219)..(219): a, c, t, g, unknown or other
  modified_base (225)..(225): a, c, t, g, unknown or other
  modified_base (240)..(240): a, c, t, g, unknown or other
  modified_base (243)..(243): a, c, t, g, unknown or other
  modified_base (249)..(249): a, c, t, g, unknown or other
  modified_base (261)..(261): a, c, t, g, unknown or other
  modified_base (267)..(267): a, c, t, g, unknown or other
  modified_base (270)..(270): a, c, t, g, unknown or other
  modified_base (285)..(285): a, c, t, g, unknown or other
  modified_base (288)..(288): a, c, t, g, unknown or other
  modified_base (291)..(291): a, c, t, g, unknown or other
  modified_base (294)..(294): a, c, t, g, unknown or other
  modified_base (303)..(303): a, c, t, g, unknown or other
  modified_base (306)..(306): a, c, t, g, unknown or other
  modified_base (309)..(309): a, c, t, g, unknown or other
  modified_base (315)..(315): a, c, t, g, unknown or other
  modified_base (321)..(321): a, c, t, g, unknown or other
  modified_base (330)..(330): a, c, t, g, unknown or other
  modified_base (339)..(339): a, c, t, g, unknown or other
  modified_base (342)..(342): a, c, t, g, unknown or other
  modified_base (348)..(348): a, c, t, g, unknown or other
  modified_base (354)..(354): a, c, t, g, unknown or other
  modified_base (360)..(360): a, c, t, g, unknown or other
  modified_base (372)..(372): a, c, t, g, unknown or other
  modified_base (375)..(375): a, c, t, g, unknown or other
  modified_base (378)..(378): a, c, t, g, unknown or other
  modified_base (381)..(381): a, c, t, g, unknown or other
  modified_base (384)..(384): a, c, t, g, unknown or other
  modified_base (387)..(387): a, c, t, g, unknown or other
  modified_base (393)..(393): a, c, t, g, unknown or other
  modified_base (402)..(402): a, c, t, g, unknown or other
  modified_base (405)..(405): a, c, t, g, unknown or other
  modified_base (408)..(408): a, c, t, g, unknown or other
  modified_base (411)..(411): a, c, t, g, unknown or other
  modified_base (420)..(420): a, c, t, g, unknown or other
  modified_base (423)..(423): a, c, t, g, unknown or other
  modified_base (426)..(426): a, c, t, g, unknown or other
  modified_base (435)..(435): a, c, t, g, unknown or other
  modified_base (438)..(438): a, c, t, g, unknown or other
  modified_base (441)..(441): a, c, t, g, unknown or other
  modified_base (444)..(444): a, c, t, g, unknown or other
  modified_base (450)..(450): a, c, t, g, unknown or other
  modified_base (456)..(456): a, c, t, g, unknown or other
  modified_base (459)..(459): a, c, t, g, unknown or other
  modified_base (483)..(483): a, c, t, g, unknown or other
  modified_base (486)..(486): a, c, t, g, unknown or other
  modified_base (489)..(489): a, c, t, g, unknown or other
  modified_base (510)..(510): a, c, t, g, unknown or other
  modified_base (513)..(513): a, c, t, g, unknown or other
  modified_base (519)..(519): a, c, t, g, unknown or other
  modified_base (525)..(525): a, c, t, g, unknown or other
  modified_base (531)..(531): a, c, t, g, unknown or other
  modified_base (537)..(537): a, c, t, g, unknown or other
  modified_base (540)..(540): a, c, t, g, unknown or other
  modified_base (543)..(543): a, c, t, g, unknown or other
  modified_base (561)..(561): a, c, t, g, unknown or other
  modified_base (564)..(564): a, c, t, g, unknown or other
  modified_base (567)..(567): a, c, t, g, unknown or other
  modified_base (570)..(570): a, c, t, g, unknown or other
  modified_base (573)..(573): a, c, t, g, unknown or other
  modified_base (582)..(582): a, c, t, g, unknown or other
  modified_base (585)..(585): a, c, t, g, unknown or other
  modified_base (588)..(588): a, c, t, g, unknown or other
  modified_base (591)..(591): a, c, t, g, unknown or other
  modified_base (594)..(594): a, c, t, g, unknown or other
  modified_base (603)..(603): a, c, t, g, unknown or other
  modified_base (606)..(606): a, c, t, g, unknown or other
  modified_base (612)..(612): a, c, t, g, unknown or other
  modified_base (618)..(618): a, c, t, g, unknown or other
  modified_base (621)..(621): a, c, t, g, unknown or other
  modified_base (624)..(624): a, c, t, g, unknown or other
  modified_base (627)..(627): a, c, t, g, unknown or other
  modified_base (630)..(630): a, c, t, g, unknown or other
  modified_base (633)..(633): a, c, t, g, unknown or other
  modified_base (639)..(639): a, c, t, g, unknown or other
  modified_base (651)..(651): a, c, t, g, unknown or other
  modified_base (654)..(654): a, c, t, g, unknown or other
  modified_base (660)..(660): a, c, t, g, unknown or other
  modified_base (663)..(663): a, c, t, g, unknown or other
  modified_base (666)..(666): a, c, t, g, unknown or other
  modified_base (684)..(684): a, c, t, g, unknown or other
  modified_base (690)..(690): a, c, t, g, unknown or other
  modified_base (696)..(696): a, c, t, g, unknown or other
  modified_base (699)..(699): a, c, t, g, unknown or other
  modified_base (705)..(705): a, c, t, g, unknown or other
  modified_base (708)..(708): a, c, t, g, unknown or other
  modified_base (711)..(711): a, c, t, g, unknown or other
  modified_base (717)..(717): a, c, t, g, unknown or other
  modified_base (720)..(720): a, c, t, g, unknown or other
  modified_base (723)..(723): a, c, t, g, unknown or other
  modified_base (726)..(726): a, c, t, g, unknown or other
  modified_base (729)..(729): a, c, t, g, unknown or other
  modified_base (732)..(732): a, c, t, g, unknown or other
  modified_base (735)..(735): a, c, t, g, unknown or other
  modified_base (741)..(741): a, c, t, g, unknown or other
  modified_base (747)..(747): a, c, t, g, unknown or other
  modified_base (750)..(750): a, c, t, g, unknown or other
  modified_base (756)..(756): a, c, t, g, unknown or other
  modified_base (765)..(765): a, c, t, g, unknown or other
  modified_base (768)..(768): a, c, t, g, unknown or other
  modified_base (777)..(777): a, c, t, g, unknown or other
  modified_base (780)..(780): a, c, t, g, unknown or other
  modified_base (783)..(783): a, c, t, g, unknown or other
  modified_base (786)..(786): a, c, t, g, unknown or other
  modified_base (792)..(792): a, c, t, g, unknown or other
  modified_base (795)..(795): a, c, t, g, unknown or other
  modified_base (801)..(801): a, c, t, g, unknown or other
  modified_base (804)..(804): a, c, t, g, unknown or other
  modified_base (807)..(807): a, c, t, g, unknown or other
  modified_base (813)..(813): a, c, t, g, unknown or other
  modified_base (816)..(816): a, c, t, g, unknown or other
  modified_base (828)..(828): a, c, t, g, unknown or other
  modified_base (834)..(834): a, c, t, g, unknown or other
  modified_base (852)..(852): a, c, t, g, unknown or other
  modified_base (858)..(858): a, c, t, g, unknown or other
  modified_base (861)..(861): a, c, t, g, unknown or other
  modified_base (867)..(867): a, c, t, g, unknown or other
  modified_base (876)..(876): a, c, t, g, unknown or other
  modified_base (882)..(882): a, c, t, g, unknown or other
  modified_base (888)..(888): a, c, t, g, unknown or other
  modified_base (891)..(891): a, c, t, g, unknown or other
  modified_base (909)..(909): a, c, t, g, unknown or other
  modified_base (912)..(912): a, c, t, g, unknown or other
  modified_base (918)..(918): a, c, t, g, unknown or other
  modified_base (921)..(921): a, c, t, g, unknown or other
  modified_base (924)..(924): a, c, t, g, unknown or other
  modified_base (927)..(927): a, c, t, g, unknown or other
  modified_base (930)..(930): a, c, t, g, unknown or other
  modified_base (933)..(933): a, c, t, g, unknown or other
  modified_base (936)..(936): a, c, t, g, unknown or other
  modified_base (939)..(939): a, c, t, g, unknown or other
  modified_base (942)..(942): a, c, t, g, unknown or other
  modified_base (957)..(957): a, c, t, g, unknown or other
  modified_base (963)..(963): a, c, t, g, unknown or other
  modified_base (984)..(984): a, c, t, g, unknown or other
  modified_base (987)..(987): a, c, t, g, unknown or other
  modified_base (996)..(996): a, c, t, g, unknown or other
  modified_base (999)..(999): a, c, t, g, unknown or other
  modified_base (1002)..(1002): a, c, t, g, unknown or other
  modified_base (1005)..(1005): a, c, t, g, unknown or other
  modified_base (1008)..(1008): a, c, t, g, unknown or other
  modified_base (1020)..(1020): a, c, t, g, unknown or other
  modified_base (1026)..(1026): a, c, t, g, unknown or other
  modified_base (1032)..(1032): a, c, t, g, unknown or other
  modified_base (1038)..(1038): a, c, t, g, unknown or other
  modified_base (1044)..(1044): a, c, t, g, unknown or other
  modified_base (1047)..(1047): a, c, t, g, unknown or other
  modified_base (1053)..(1053): a, c, t, g, unknown or other
  modified_base (1059)..(1059): a, c, t, g, unknown or other
  modified_base (1065)..(1065): a, c, t, g, unknown or other
  modified_base (1068)..(1068): a, c, t, g, unknown or other
  modified_base (1071)..(1071): a, c, t, g, unknown or other
  modified_base (1074)..(1074): a, c, t, g, unknown or other
  modified_base (1077)..(1077): a, c, t, g, unknown or other
  modified_base (1080)..(1080): a, c, t, g, unknown or other
  modified_base (1089)..(1089): a, c, t, g, unknown or other
  modified_base (1092)..(1092): a, c, t, g, unknown or other
  modified_base (1104)..(1104): a, c, t, g, unknown or other
  modified_base (1107)..(1107): a, c, t, g, unknown or other
  modified_base (1110)..(1110): a, c, t, g, unknown or other
  modified_base (1113)..(1113): a, c, t, g, unknown or other
  modified_base (1119)..(1119): a, c, t, g, unknown or other
  modified_base (1122)..(1122): a, c, t, g, unknown or other
  modified_base (1128)..(1128): a, c, t, g, unknown or other
  modified_base (1137)..(1137): a, c, t, g, unknown or other
  modified_base (1140)..(1140): a, c, t, g, unknown or other
  modified_base (1149)..(1149): a, c, t, g, unknown or other
  modified_base (1152)..(1152): a, c, t, g, unknown or other
  modified_base (1164)..(1164): a, c, t, g, unknown or other
  modified_base (1170)..(1170): a, c, t, g, unknown or other
  modified_base (1176)..(1176): a, c, t, g, unknown or other
  modified_base (1194)..(1194): a, c, t, g, unknown or other
  modified_base (1197)..(1197): a, c, t, g, unknown or other
  modified_base (1200)..(1200): a, c, t, g, unknown or other
  modified_base (1203)..(1203): a, c, t, g, unknown or other
  modified_base (1206)..(1206): a, c, t, g, unknown or other
  modified_base (1209)..(1209): a, c, t, g, unknown or other
  modified_base (1215)..(1215): a, c, t, g, unknown or other
  modified_base (1221)..(1221): a, c, t, g, unknown or other
  modified_base (1224)..(1224): a, c, t, g, unknown or other
  modified_base (1233)..(1233): a, c, t, g, unknown or other
  modified_base (1239)..(1239): a, c, t, g, unknown or other
  modified_base (1245)..(1245): a, c, t, g, unknown or other
  modified_base (1248)..(1248): a, c, t, g, unknown or other
  modified_base (1251)..(1251): a, c, t, g, unknown or other
  modified_base (1260)..(1260): a, c, t, g, unknown or other
  modified_base (1263)..(1263): a, c, t, g, unknown or other
  modified_base (1275)..(1275): a, c, t, g, unknown or other
  modified_base (1281)..(1281): a, c, t, g, unknown or other
  modified_base (1287)..(1287): a, c, t, g, unknown or other
  modified_base (1293)..(1293): a, c, t, g, unknown or other
  modified_base (1296)..(1296): a, c, t, g, unknown or other
  modified_base (1308)..(1308): a, c, t, g, unknown or other
  modified_base (1311)..(1311): a, c, t, g, unknown or other
  modified_base (1326)..(1326): a, c, t, g, unknown or other
  modified_base (1335)..(1335): a, c, t, g, unknown or other
  modified_base (1338)..(1338): a, c, t, g, unknown or other
  modified_base (1341)..(1341): a, c, t, g, unknown or other
  modified_base (1344)..(1344): a, c, t, g, unknown or other
  modified_base (1347)..(1347): a, c, t, g, unknown or other
  modified_base (1350)..(1350): a, c, t, g, unknown or other
  modified_base (1353)..(1353): a, c, t, g, unknown or other
atgtaymgnatgcarytnytnytnytnathgcnytnwsnytngcnytngtnacnaaywsnwsngayacnggnmgnccnttygtngaratgtaywsngarathccngarathathcayatgacngarggnmgngarytngtnathccntgymgngtnacnwsnccnaayathacngtnacnytnaaraarttyccnytngayacnytnathccngayggnaarmgnathathtgggaywsnmgnaarggnttyathathwsnaaygcnacntayaargarathggnytnytnacntgygargcnacngtnaayggncayytntayaaracnaaytayytnacncaymgncaracnaayacnathathgaygtngtnytnwsnccnwsncayggnathgarytnwsngtnggngaraarytngtnytnaaytgyacngcnmgnacngarytnaaygtnggnathgayttyaaytgggartayccnwsnwsnaarcaycarcayaaraarytngtnaaymgngayytnaaracncarwsnggnwsngaratgaaraarttyytnwsnacnytnacnathgayggngtnacnmgnwsngaycarggnytntayacntgygcngcnwsnwsnggnytnatgacnaaraaraaywsnacnttygtnmgngtncaygaraargayaaracncayacntgyccnccntgyccngcnccngarytnytnggnggnccnwsngtnttyytnttyccnccnaarccnaargayacnytnatgathwsnmgnacnccngargtnacntgygtngtngtngaygtnwsncaygargayccngargtnaarttyaaytggtaygtngayggngtngargtncayaaygcnaaracnaarccnmgngargarcartayaaywsnacntaymgngtngtnwsngtnytnacngtnytncaycargaytggytnaayggnaargartayaartgyaargtnwsnaayaargcnytnccngcnccnathgaraaracnathwsnaargcnaarggncarccnmgngarccncargtntayacnytnccnccnwsnmgngaygarytnacnaaraaycargtnwsnytnacntgyytngtnaarggnttytayccnwsngayathgcngtngartgggarwsnaayggncarccngaraayaaytayaaracnacnccnccngtnytngaywsngayggnwsnttyttyytntaywsnaarytnacngtngayaarwsnmgntggcarcarggnaaygtnttywsntgywsngtnatgcaygargcnytncayaaycaytayacncaraarwsnytnwsnytnwsnccnggn

SEQ ID NO: 4 - protein, 736 aa, Adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 5 - protein, 735 aa, Adeno-associated virus 2 [sequence omitted]

SEQ ID NO: 6 - protein, 736 aa, Adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 7 - protein, 734 aa, Adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 8 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 9 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 10 - protein, 737 aa, Adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 11 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 12 - protein, 736 aa, Adeno-associated virus
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 13 - protein, 736 aa, Adeno-associated virus
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 457 aa, Artificial Sequence
  source: /note=“Description of Artificial Sequence Synthetic polypeptide”
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 451 aa, Artificial Sequence
  source: /note=“Description of Artificial Sequence Synthetic polypeptide”
MYRMQLLLLIALSLALVTNSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 451 aa, Artificial Sequence; identical to SEQ ID NO: 16
  source: /note=“Description of Artificial Sequence Synthetic polypeptide”
  VARIANT (440)..(440): /replace=“Ala” or “Gln”
  SITE (1)..(451): /note=“Variant residues given in the sequence have no preference with respect to those in the annotations for variant positions”

SEQ ID NO: 18 - protein, 247 aa, Artificial Sequence
  source: /note=“Description of Artificial Sequence Synthetic polypeptide”
  VARIANT (225)..(247): /replace=“ ”
  VARIANT (226)..(247): /replace=“ ”
  VARIANT (230)..(230): /replace=“Leu”
  VARIANT (231)..(247): /replace=“ ”
  VARIANT (237)..(247): /replace=“ ”
  VARIANT (243)..(247): /replace=“ ”
  SITE (1)..(247): /note=“Variant residues given in the sequence have no preference with respect to those in the annotations for variant positions”
MYRMQLLLLIALSLALVTNSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFL

SEQ ID NO: 19 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 20 - protein, 327 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 428 aa, Artificial Sequence
  source: /note=“Description of Artificial Sequence Synthetic polypeptide”
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 22 - protein, 433 aa, Artificial Sequence
  source: /note=“Description of Artificial Sequence Synthetic polypeptide”
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 23 - protein, 421 aa, Artificial Sequence
  source: /note=“Description of Artificial Sequence Synthetic polypeptide”
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKWESNGQPENNYKTTPPVLDSDGSFFLYSRLTVDKSRWQEGNVFSCSVMHEALHNHYTQKSLSLSLGK

SEQ ID NO: 24 - protein, 421 aa, Artificial Sequence
  source: /note=“Description of Artificial Sequence Synthetic polypeptide”
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKYGPPSPSSPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKWESNGQPENNYKTTPPVLDSDGSFFLYSRLTVDKSRWQEGNVFSCSVMHEALHNHYTQKSLSLSLGK

SEQ ID NO: 25 - protein, 434 aa, Artificial Sequence
  source: /note=“Description of Artificial Sequence Synthetic polypeptide”
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 434 aa, Artificial Sequence
  source: /note=“Description of Artificial Sequence Synthetic polypeptide”
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKESKYGPPSPSSPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 758 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLE

SEQ ID NO: 28 - protein, 764 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 29 - protein, 409 aa, Artificial Sequence
  source: /note=“Description of Artificial Sequence Synthetic polypeptide”
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVK

SEQ ID NO: 30 - protein, 426 aa, Artificial Sequence
  source: /note=“Description of Artificial Sequence Synthetic polypeptide”
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVT

SEQ ID NO: 31 - protein, 6 aa, Artificial Sequence
  source: /note=“Description of Artificial Sequence Synthetic peptide”
KDKTHT

SEQ ID NO: 32 - protein, 6 aa, Artificial Sequence
  source: /note=“Description of Artificial Sequence Synthetic peptide”
KDKTHL

SEQ ID NO: 33 - protein, 12 aa, Artificial Sequence
  source: /note=“Description of Artificial Sequence Synthetic peptide”
KDKTHTCPPCPA

SEQ ID NO: 34 - protein, 18 aa, Artificial Sequence
  source: /note=“Description of Artificial Sequence Synthetic peptide”
KDKTHTCPPCPAPELLGG

SEQ ID NO: 35 - protein, 23 aa, Artificial Sequence
  source: /note=“Description of Artificial Sequence Synthetic peptide”
KDKTHTCPPCPAPELLGGPSVFL

SEQ ID NO: 36 - protein, 26 aa, Homo sapiens
MVSYWDTGVLLCALLSCLLLTGSSSG

SEQ ID NO: 37 - protein, 19 aa, Homo sapiens
MQSKVLLAVALWLCVETRA

SEQ ID NO: 38 - protein, 20 aa, Unknown
  source: /note=“Description of Unknown heterologous leader sequence”
MYRMQLLLLIALSLALVTNS

SEQ ID NO: 39 - protein, 19 aa, Unknown
  source: /note=“Description of Unknown heterologous leader sequence”
MRMQLLLLIALSLALVTNS

SEQ ID NO: 40 - protein, 8 aa, Artificial Sequence
  source: /note=“Description of Artificial Sequence Synthetic peptide”
  SITE (1)..(8): /note=“This sequence may encompass 1-4 ′Gly Pro′ repeating units”
GPGPGPGP

SEQ ID NO: 41 - protein, 8 aa, Artificial Sequence
  source: /note=“Description of Artificial Sequence Synthetic peptide”
  SITE (1)..(8): /note=“This sequence may encompass 1-4 ′Ala Pro′ repeating units”
APAPAPAP

SEQ ID NO: 42 - protein, 15 aa, Artificial Sequence
  source: /note=“Description of Artificial Sequence Synthetic peptide”
EAAAKEAAAKEAAAK

SEQ ID NO: 43 - protein, 20 aa, Artificial Sequence
  source: /note=“Description of Artificial Sequence Synthetic peptide”
  SITE (1)..(20): /note=“This sequence may encompass 1-4 ′Gly Gly Gly Gly Ser′ repeating units”
GGGGSGGGGSGGGGSGGGGS

SEQ ID NO: 44 - protein, 5 aa, Artificial Sequence
  source: /note=“Description of Artificial Sequence Synthetic peptide”
DKTHT

SEQ ID NO: 45 - protein, 5 aa, Artificial Sequence
  source: /note=“Description of Artificial Sequence Synthetic peptide”
DKTHL

SEQ ID NO: 46 - protein, 11 aa, Artificial Sequence
  source: /note=“Description of Artificial Sequence Synthetic peptide”
DKTHTCPPCPA

SEQ ID NO: 47 - protein, 17 aa, Artificial Sequence
  source: /note=“Description of Artificial Sequence Synthetic peptide”
DKTHTCPPCPAPELLGG

SEQ ID NO: 48 - protein, 22 aa, Artificial Sequence
  source: /note=“Description of Artificial Sequence Synthetic peptide”
DKTHTCPPCPAPELLGGPSVFL

SEQ ID NO: 49 - protein, 223 aa, Artificial Sequence
  source: /note=“Description of Artificial Sequence Synthetic polypeptide”
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 50 - protein, 228 aa, Artificial Sequence
  source: /note=“Description of Artificial Sequence Synthetic polypeptide”
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 51 - protein, 226 aa, Artificial Sequence
  source: /note=“Description of Artificial Sequence Synthetic polypeptide”
YGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - protein, 226 aa, Artificial Sequence
  source: /note=“Description of Artificial Sequence Synthetic polypeptide”
YGPPSPSSPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 229 aa, Artificial Sequence
  source: /note=“Description of Artificial Sequence Synthetic polypeptide”
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 229 aa, Artificial Sequence
  source: /note=“Description of Artificial Sequence Synthetic polypeptide”
ESKYGPPSPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 204 aa, Homo sapiens
PFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVK

SEQ ID NO: 56 - protein, 221 aa, Homo sapiens
PFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVT

SEQ ID NO: 57 - protein, 7 aa, Adeno-associated virus
LGETTRP

SEQ ID NO: 58 - protein, 9 aa, Adeno-associated virus
LALGETTRP

SEQ ID NO: 59 - protein, 26 aa, Unknown
  source: /note=“Description of Unknown VEGF-A signal peptide”
MNFLLSWVHWSLALLLYLHHAKWSQA

SEQ ID NO: 60 - protein, 29 aa, Unknown
  source: /note=“Description of Unknown Fibulin-1 signal peptide”
MERAAPSRRVPLPLLLLGGLALLAAGVDA

SEQ ID NO: 61 - protein, 19 aa, Unknown
  source: /note=“Description of Unknown Vitronectin signal peptide”
MAPLRPLLILALLAWVALA

SEQ ID NO: 62 - protein, 18 aa, Unknown
  source: /note=“Description of Unknown Complement Factor H signal peptide”
MRLLAKIICLMLWAICVA

SEQ ID NO: 63 - protein, 19 aa, Unknown
  source: /note=“Description of Unknown Opticin signal peptide”
MRLLAFLSLLALVLQETGT

SEQ ID NO: 64 - protein, 18 aa, Unknown
  source: /note=“Description of Unknown Albumin signal peptide”
MKWVTFISLLFLFSSAYS

SEQ ID NO: 65 - protein, 18 aa, Unknown
  source: /note=“Description of Unknown Chymotrypsinogen signal peptide”
MAFLWLLSCWALLGTTFG

SEQ ID NO: 66 - protein, 20 aa, Unknown
  source: /note=“Description of Unknown Interleukin-2 signal peptide”
MYRMQLLSCIALILALVTNS

SEQ ID NO: 67 - protein, 15 aa, Unknown
  source: /note=“Description of Unknown Trypsinogen-2 signal peptide”
MNLLLILTFVAAAVA

SEQ ID NO: 68 - protein, 24 aa, Homo sapiens
MPSSVSWGILLLAGLCCLVPVSLA

SEQ ID NO: 69 - protein, 18 aa, Homo sapiens
MKAAVLTLAVLFLTGSQA

SEQ ID NO: 70 - protein, 18 aa, Homo sapiens
MKLLAATVLLLTICSLEG

SEQ ID NO: 71 - protein, 27 aa, Homo sapiens
MDPPRPALLALLALPALLLLLLAGARA

SEQ ID NO: 72 - protein, 28 aa, Homo sapiens
MQRVNMIMAESPGLITICLLGYLLSAEC

SEQ ID NO: 73 - protein, 20 aa, Homo sapiens
MGPLMVLFCLLFLYPGLADS

SEQ ID NO: 74 - protein, 18 aa, Homo sapiens
MWLLVSVILISRISSVGG

SEQ ID NO: 75 - protein, 18 aa, Homo sapiens
MLLLFSVILISWVSTVGG

SEQ ID NO: 76 - protein, 19 aa, Homo sapiens
MFSMRIVCLVLSVVGTAWT

SEQ ID NO: 77 - protein, 30 aa, Homo sapiens
MKRMVSWSFHKLKTMKHLLLLLLCVFLVKS

SEQ ID NO: 78 - protein, 26 aa, Homo sapiens
MSWSLHPRNLILYFYALLFLSSTCVA

SEQ ID NO: 79 - protein, 18 aa, Homo sapiens
MKSLVLLLCLAQLWGCHS

SEQ ID NO: 80 - protein, 23 aa, Homo sapiens
MARVLGAPVALGLWSLCWSLAIA

SEQ ID NO: 81 - protein, 18 aa, Homo sapiens
MKLITILFLCSRLLLSLT

SEQ ID NO: 82 - protein, 20 aa, Homo sapiens
MSLFPSLPLLLLSMVAASYS

SEQ ID NO: 83 - protein, 19 aa, Homo sapiens
MEHKEVVLLLLLFLKSGQG

SEQ ID NO: 84 - protein, 24 aa, Homo sapiens
MAHVRGLQLPGCLALAALCSLVHS

SEQ ID NO: 85 - protein, 29 aa, Homo sapiens
MISRMEKMTMMMKILIMFALGMNYWSCSG

SEQ ID NO: 86 - protein, 32 aa, Homo sapiens
MYSNVIGTVTSGKRKVYLLSLLLIGFWDCVTC

SEQ ID NO: 87 - protein, 19 aa, Homo sapiens
MRLAVGALLVCAVLGLCLA

SEQ ID NO: 88 - protein, 19 aa, Artificial Sequence
  source: /note=“Description of Artificial Sequence Synthetic peptide”
LLNFDLLKLAGDVESNPGP

SEQ ID NO: 89 - protein, 21 aa, Artificial Sequence
  source: /note=“Description of Artificial Sequence Synthetic peptide”
  VARIANT (1)..(3): /replace=“ ”
  SITE (1)..(21): /note=“Variant residues given in the sequence have no preference with respect to those in the annotations for variant positions”
GSGEGRGSLLTCGDVEENPGP

SEQ ID NO: 90 - protein, 22 aa, Artificial Sequence
  source: /note=“Description of Artificial Sequence Synthetic peptide”
  VARIANT (1)..(3): /replace=“ ”
  SITE (1)..(22): /note=“Variant residues given in the sequence have no preference with respect to those in the annotations for variant positions”
GSGATNFSLLKQAGDVEENPGP

SEQ ID NO: 91 - protein, 23 aa, Artificial Sequence
  source: /note=“Description of Artificial Sequence Synthetic peptide”
  VARIANT (1)..(3): /replace=“ ”
  SITE (1)..(23): /note=“Variant residues given in the sequence have no preference with respect to those in the annotations for variant positions”
GSGQCTNYALLKLAGDVESNPGP

SEQ ID NO: 92 - protein, 25 aa, Artificial Sequence
  source: /note=“Description of Artificial Sequence Synthetic peptide”
  VARIANT (1)..(3): /replace=“ ”
  SITE (1)..(25): /note=“Variant residues given in the sequence have no preference with respect to those in the annotations for variant positions”
GSGVKQTLNFDLLKLAGDVESNPGP

SEQ ID NO: 93 - protein, 4 aa, Artificial Sequence
  source: /note=“Description of Artificial Sequence Synthetic peptide”
RKRR

SEQ ID NO: 94 - protein, 4 aa, Artificial Sequence
  source: /note=“Description of Artificial Sequence Synthetic peptide”
RRRR

SEQ ID NO: 95 - protein, 4 aa, Artificial Sequence
  source: /note=“Description of Artificial Sequence Synthetic peptide”
RRKR

SEQ ID NO: 96 - protein, 4 aa, Artificial Sequence
  source: /note=“Description of Artificial Sequence Synthetic peptide”
RKKR

SEQ ID NO: 97 - protein, 18 aa, Homo sapiens
MKWVTFISLLFLFSSAYS